(19) United States
(12) Patent Application Publication
(10) Pub. No.: US 2025/0387403 A1
(43) Pub. Date: Dec. 25, 2025

(54) SELECTIVE SIGMA-2 RECEPTOR LIGANDS AS MODULATORS OF TMEM97

(71) Applicant: BOARD OF REGENTS, THE UNIVERSITY OF TEXAS SYSTEM, Austin, TX (US)

(72) Inventors: Stephen F. MARTIN, Austin, TX (US); James J. SAHN, Austin, TX (US)

(21) Appl. No.: 18/866,240

(22) PCT Filed: May 17, 2023
(86) PCT No.: PCT/US2023/067129

§ 371 Date: Nov. 15, 2024

Related U.S. Application Data
(60) Provisional application No. 63/343,070, filed on May 17, 2022.

(51) Int. Cl. A61K 31/55; C07D 471/08; C07D 487/08; C07D 519/00

(52) U.S. Cl. CPC A61K 31/55; C07D 471/08; C07D 487/08; C07D 519/00

(57) ABSTRACT

The present disclosure relates to compounds that may be used to modulate the activity of a σ2R/TMEM97 receptor. The compounds may be further defined by the formula: (IA), (IB), or (IC) wherein the variables are as described herein. The present disclosure also provides pharmaceutical compositions of the compounds. Also, provided herein are methods of using the compounds in the treatment of neurodegenerative diseases or disorders among other indications.

The present disclosure claims the benefit of priority to U.S. Provisional Application No. 63/343,070, filed on May 17, 2022, the entire contents of which are hereby incorporated by reference.

### REFERENCE TO A SEQUENCE LISTING

This application contains a Sequence Listing XML, which has been submitted electronically and is hereby incorporated by reference in its entirety. Said Sequence Listing XML, created on May 16, 2023, is named UTFBP1309WO.xml and is ˜4 kilobytes in size.

### BACKGROUND

### 1. Field

The present disclosure relates generally to the fields of medicine, pharmaceutical agents, and sigma receptor binders. The present disclosure relates to compounds that may be used to modulate the activity of one or more sigma receptors.

### 2. Description of Related Art

Small molecules have a rich history in drug discovery because of their ability to selectively target and inhibit or activate proteins involved in pathogenic pathways. In this context, compounds that bind to sigma receptors (σRs) are gaining prominence (Schmidt and Kruse, 2019; Smith, 2017). The sigma 1 receptor (σ₁R), which shows no homology with any other mammalian protein, is located in the endoplasmic reticulum (ER) where it is enriched in the mitochondria-associated membrane subregion and is involved in calcium modulation (Pontisso and Combettes, 2021; Hayashi and Su, 2007). Small molecules that bind to the σ₁R have been shown to exhibit promising attributes in neurodegenerative and neurological disorders, (Hayashi 2015; Nguyen et al, 2015; Nguyen et al, 2017; Ryskamp et al, 2019) and several σ₁R ligands have been shown to have neuroprotective effects in animal models of HD (Hyrskyluoto et al, 2013; de Yebenes et al, 2011; Ryskamp et al, 2017), including pridopidine that is in human clinical trials (Squitieri et al, 2015; Geva et al, 2016; Sahlholm et al, 2018; Eddings et al, 2019).

The sigma 2 receptor (σ₂R), which is biochemically distinct from σ₁R, was initially associated with cancer diagnosis and therapy (Mach et al, 2013; Huang et al, 2014; Qiu et al, 2015; Zeng and Mach, 2017) but more recently it has been implicated in neurological disorders (Izzo et al, 2014; Guo and Zhen, 2015). The molecular identity of σ₂R was an enigma from its discovery until a few years ago, when it was cloned and identified as the endoplasmic reticulum-resident transmembrane protein 97 (TMEM97) (Alon et al, 2017), herein referred to as σ₂R/TMEM97. Although the biological function of σ₂R/TMEM97 is not well characterized, it is known to play a role in cholesterol trafficking and homeostasis (Bartz et al, 2009), and it appears to be a binding partner of the lysosomal cholesterol transporter NPC1 (Ebrahimi-Fakhari et al, 2016), a mutation in which results in Niemann-Pick disease type C. Small molecules that modulate σ₂R/TMEM97 signaling show beneficial effects in different disease contexts, including cancer (Liu et al, 2019; Yang et al, 2020), neuropathic pain (Sahn et al, 2017), traumatic brain injury (Vazquez-Rosa et al, 2019), alcohol use disorder (Scott et al, 2018; Quadir et al, 2021), and Alzheimer's disease (AD) (Mondal et al, 2018; Riad et al, 2020; Yi et al, 2017; Izzo et al, 2014). Moreover, a putative σ₂R/TMEM97 antagonist is in Phase II clinical trials for treating AD (Grundman et al, 2019).

The finding that modulating σ₂R/TMEM97 exhibits neuroprotection in several models of neurodegenerative disease prompted exploration as to whether compounds that bind to σ₂R/TMEM97 might provide beneficial effects in an HD model.

### SUMMARY

In some aspects, the present disclosure provides compounds that may be used as modulator of sigma receptors, in particular the sigma 2 receptor, which is identical to transmembrane protein 97 (TMEM97).

In some aspects, the present disclosure provides compounds of the formula:

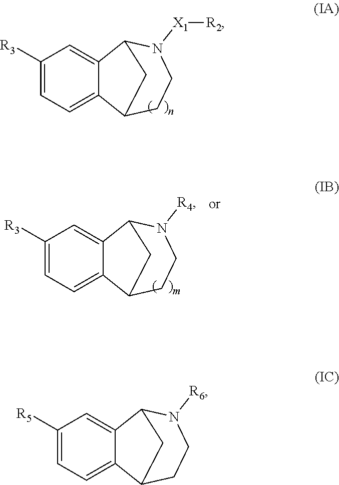

wherein:
m and n are each independently 1 or 2;
R₁ is —Z₁(R₁′)ₐ, wherein:
   Z₁ is arenediyl_{(C≤18)}, substituted arenediyl_{(C≤18)}, heteroarenediyl_{(C≤18)}, substituted heteroarenediyl_{(C≤18)}, when a is 1; arenetriyl_{(C≤18)}, substituted arenetriyl_{(C≤18)}, heteroarenetriyl_{(C≤18)}, substituted heteroarenetriyl_{(C≤18)}, when a is 2; arenequadyl_{(C≤18)}, substituted arenequadyl_{(C≤18)}, heteroarenequadyl_{(C≤18)}, substituted heteroarenequadyl_{(C≤18)}, when a is 3; or arenepentayl_{(C≤18)}, substituted arenepentayl_{(C≤18)}, heteroarenepentayl_{(C≤18)}, substituted heteroarenepentayl_{(C≤18)}, when a is 4;
   a is 1, 2, 3, or 4; and
   R₁′ is hydrogen, —C(O)R′, —OR′, —S(O)_{y}R′, —N(R′)R″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
      R′ and R″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof; and
      y is 0, 1, or 2;
X₁ is —C(O)Y₁— or —S(O)ₓY₁—, wherein:
   x is 0, 1, or 2; and
   Y₁ is a covalent bond; —O—; —NRₐ—, wherein Rₐ is hydrogen, alkyl_{(C≤6)}, or substituted alkyl_{(C≤6)}; alkanediyl_{(C≤8)}; or substituted alkanediyl_{(C≤8)}; and
R₂ is —Y₂N(R_{b})R_{c}, wherein:
   Y₂ is alkanediyl_{(C≤8)}, cycloalkanediyl_{(C≤8)}, alkenediyl_{(C≤8)}, alkynediyl_{(C≤8)}, arenediyl_{(C≤8)}, heterocycloalkanediyl_{(C≤8)}, heteroarenediyl_{(C≤8)}, or a substituted version thereof;
   R_{b} and R_{c} are each independently hydrogen, alkyl_{(C≤12)}, cycloalkyl_{(C≤12)}, aryl_{(C≤12)}, heteroaryl_{(C≤12)}, heterocycloalkyl_{(C≤12)}, acyl_{(C≤12)}, or a substituted version thereof; or
R₂ is —Y₃R_{d}, wherein:
   Y₃ is heterocycloalkanediyl_{(C≤8)}, heteroarenediyl_{(C≤8)}, or a substituted version thereof; and
   R_{d} is hydrogen, alkyl_{(C≤12)}, alkenyl_{(C≤12)}, alkynyl_{(C≤12)}, aryl_{(C≤12)}, heteroaryl_{(C≤12)}, heterocycloalkyl_{(C≤12)}, or a substituted version thereof;
R₃ is —Y₄(Rₑ)_{b}, wherein:
   Y₄ is arenediyl_{(C≤18)}, substituted arenediyl_{(C≤18)}, heteroarenediyl_{(C≤18)}, substituted heteroarenediyl_{(C≤18)}, wherein b is 1; arenetriyl_{(C≤18)}, substituted arenetriyl_{(C≤18)}, heteroarenetriyl_{(C≤18)}, substituted heteroarenetriyl_{(C≤18)}, wherein b is 2; arenequadyl_{(C≤18)}, substituted arenequadyl_{(C≤18)}, heteroarenequadyl_{(C≤18)}, substituted heteroarenequadyl_{(C≤18)}, wherein b is 3; or arenepentayl_{(C≤18)}, substituted arenepentayl_{(C≤18)}, heteroarenepentayl_{(C≤18)}, substituted heteroarenepentayl_{(C≤18)}, wherein b is 4;
   b is 1, 2, 3, or 4; and
   Rₑ is hydrogen, —C(O)Rₑ′, —ORₑ′, —S(O)_{y}Rₑ′, —N(Rₑ′)Rₑ″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
      y is 0, 1, or 2; and
      Rₑ′ and Rₑ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
R₄ is —Y₅N(R_{f})R_{g}, wherein:
   Y₅ is cycloalkanediyl_{(C≤12)}, heterocycloalkyl_{(C≤12)}, or a substituted version of either group;
   R_{f} and R_{g} are each independently hydrogen, alkyl_{(C≤12)}, cycloalkyl_{(C≤12)}, aryl_{(C≤12)}, heteroaryl_{(C≤12)}, heterocycloalkyl_{(C≤12)}, acyl_{(C≤12)}, or a substituted version thereof; or
R₄ is —Y₆R₇, wherein:
   Y₆ is alkanediyl_{(C≤12)} or a substituted version thereof; and
   R₇ is —C(O)Rₕ′, —S(O)_{z}Rₕ′, —N(Rₕ′)Rₕ″, heterocycloalkyl_{(C≤12)}, or substituted heterocycloalkyl_{(C≤12)}; wherein:
      z is 0, 1, or 2; and
      Rₕ′ and Rₕ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
R₅ is —Y₇(Rᵢ)_{c}, wherein:
   Y₇ is arenediyl_{(C≤18)}, substituted arenediyl_{(C≤18)}, heteroarenediyl_{(C≤18)}, substituted heteroarenediyl_{(C≤18)}, wherein c is 1, arenetriyl_{(C≤18)}, substituted arenetriyl_{(C≤18)}, heteroarenetriyl_{(C≤18)}, substituted heteroarenetriyl_{(C≤18)}, wherein c is 2; arenequadyl_{(C≤18)}, substituted arenequadyl_{(C≤18)}, heteroarenequadyl_{(C≤18)}, substituted heteroarenequadyl_{(C≤18)}, wherein c is 3; or arenepentayl_{(C≤18)}, substituted arenepentayl_{(C≤18)}, heteroarenepentayl_{(C≤18)}, substituted heteroarenepentayl_{(C≤18)}, wherein c is 4;
   c is 1, 2, 3, or 4; and
   Rᵢ is hydrogen, —C(O)Rᵢ′, —ORᵢ′, —S(O)ᵥRᵢ′, —N(Rᵢ′)Rᵢ″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
      v is 0, 1, or 2; and
      Rᵢ′ and Rᵢ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
R₆ is —Y₇R₈, wherein:
   Y₇ is alkanediyl_{(C≤12)} or a substituted version thereof; and
   R₈ is —ORⱼ′, wherein:
      Rⱼ′ is hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
         or a pharmaceutically acceptable salt thereof.

In some embodiments, the compounds are further defined as:

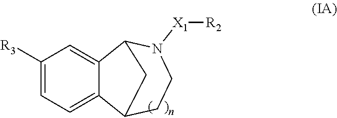

wherein:
n is 1 or 2;
R₁ is —Z₁(R₁′)ₐ, wherein:
   Z₁ is arenediyl_{(C≤18)}, substituted arenediyl_{(C≤18)}, heteroarenediyl_{(C≤18)}, substituted heteroarenediyl_{(C≤18)}, when a is 1; arenetriyl_{(C≤18)}, substituted arenetriyl_{(C≤18)}, heteroarenetriyl_{(C≤18)}, substituted heteroarenetriyl_{(C≤18)}, when a is 2; arenequadyl_{(C≤18)}, substituted arenequadyl_{(C≤18)}, heteroarenequadyl_{(C≤18)}, substituted heteroarenequadyl_{(C≤18)}, when a is 3; or arenepentayl_{(C≤18)}, substituted arenepentayl_{(C≤18)}, heteroarenepentayl_{(C≤18)}, substituted heteroarenepentayl_{(C≤18)}, when a is 4;
   a is 1, 2, 3, or 4; and
   R₁′ is hydrogen, —C(O)R′, —OR′, —S(O)_{y}R′, —N(R′)R″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
      R′ and R″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof; and
      y is 0, 1, or 2;
X₁ is —C(O)Y₁— or —S(O)ₓY₁—, wherein:
   x is 0, 1, or 2; and
   Y₁ is a covalent bond, —O—; —NRₐ—, wherein Rₐ is hydrogen, alkyl_{(C≤6)}, or substituted alkyl_{(C≤6)}; alkanediyl_{(C≤8)}; or substituted alkanediyl_{(C≤8)}; and
R₂ is —Y₂N(R_{b})R_{c}, wherein:
   Y₂ is alkanediyl_{(C≤8)}, cycloalkanediyl_{(C≤8)}, alkenediyl_{(C≤8)}, alkynediyl_{(C≤8)}, arenediyl_{(C≤8)}, heterocycloalkanediyl_{(C≤8)}, heteroarenediyl_{(C≤8)}, or a substituted version thereof;
   R_{b} and R_{c} are each independently hydrogen, alkyl_{(C≤12)}, cycloalkyl_{(C≤12)}, aryl_{(C≤12)}, heteroaryl_{(C≤12)}, heterocycloalkyl_{(C≤12)}, acyl_{(C≤12)}, or a substituted version thereof; or
R₂ is —Y₃R_{d}, wherein:
   Y₃ is heterocycloalkanediyl_{(C≤8)}, heteroarenediyl_{(C≤8)}, or a substituted version thereof; and
   R_{d} is hydrogen, alkyl_{(C≤12)}, alkenyl_{(C≤12)}, alkynyl_{(C≤12)}, aryl_{(C≤12)}, heteroaryl_{(C≤12)}, heterocycloalkyl_{(C≤12)}, or a substituted version thereof;
      or a pharmaceutically acceptable salt thereof.

In some embodiments, the compounds are further defined as:

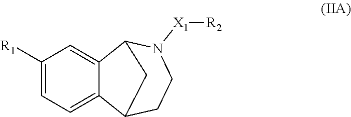

wherein:
R₁ is —Z₁(R₁′)ₐ, wherein:
   Z₁ is arenediyl_{(C≤18)}, substituted arenediyl_{(C≤18)}, heteroarenediyl_{(C≤18)}, substituted heteroarenediyl_{(C≤18)}, when a is 1; arenetriyl_{(C≤18)}, substituted arenetriyl_{(C≤18)}, heteroarenetriyl_{(C≤18)}, substituted heteroarenetriyl_{(C≤18)}, when a is 2; arenequadyl_{(C≤18)}, substituted arenequadyl_{(C≤18)}, heteroarenequadyl_{(C≤18)}, substituted heteroarenequadyl_{(C≤18)}, when a is 3; or arenepentayl_{(C≤18)}, substituted arenepentayl_{(C≤18)}, heteroarenepentayl_{(C≤18)}, substituted heteroarenepentayl_{(C≤18)}, when a is 4;
   a is 1, 2, 3, or 4; and
   R₁′ is hydrogen, —C(O)R′, —OR′, —S(O)_{y}R′, —N(R′)R″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
      R′ and R″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof; and
      y is 0, 1, or 2;
X₁ is —C(O)Y₁— or —S(O)ₓY₁—, wherein:
   x is 0, 1, or 2; and
   Y₁ is a covalent bond, —O—; —NRₐ—, wherein Rₐ is hydrogen, alkyl_{(C≤6)}, or substituted alkyl_{(C≤6)}; alkanediyl_{(C≤8)}; or substituted alkanediyl_{(C≤8)}; and
R₂ is —Y₂N(R_{b})R_{c}, wherein:
   Y₂ is alkanediyl_{(C≤8)}, cycloalkanediyl_{(C≤8)}, alkenediyl_{(C≤8)}, alkynediyl_{(C≤8)}, arenediyl_{(C≤8)}, heterocycloalkanediyl_{(C≤8)}, heteroarenediyl_{(C≤8)}, or a substituted version thereof;
   R_{b} and R_{c} are each independently hydrogen, alkyl_{(C≤12)}, cycloalkyl_{(C≤12)}, aryl_{(C≤12)}, heteroaryl_{(C≤12)}, heterocycloalkyl_{(C≤12)}, acyl_{(C≤12)}, or a substituted version thereof; or
R₂ is —Y₃R_{d}, wherein:
   Y₃ is heterocycloalkanediyl_{(C≤8)}, heteroarenediyl_{(C≤8)}, or a substituted version thereof; and
   R_{d} is hydrogen, alkyl_{(C≤12)}, alkenyl_{(C≤12)}, alkynyl_{(C≤12)}, aryl_{(C≤12)}, heteroaryl_{(C≤12)}, heterocycloalkyl_{(C≤12)}, or a substituted version thereof;
      or a pharmaceutically acceptable salt thereof.

In some embodiments, the compounds are further defined as:

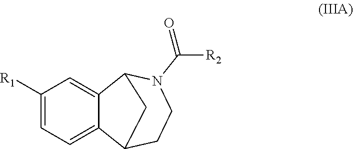

wherein:
R₁ is —Z₁(R₁′)ₐ, wherein:
   Z₁ is arenediyl_{(C≤18)}, substituted arenediyl_{(C≤18)}, heteroarenediyl_{(C≤18)}, substituted heteroarenediyl_{(C≤18)}, when a is 1; arenetriyl_{(C≤18)}, substituted arenetriyl_{(C≤18)}, heteroarenetriyl_{(C≤18)}, substituted heteroarenetriyl_{(C≤18)}, when a is 2; arenequadyl_{(C≤18)}, substituted arenequadyl_{(C≤18)}, heteroarenequadyl_{(C≤18)}, substituted heteroarenequadyl_{(C≤18)}, when a is 3; or arenepentayl_{(C≤18)}, substituted arenepentayl_{(C≤18)}, heteroarenepentayl_{(C≤18)}, substituted heteroarenepentayl_{(C≤18)}, when a is 4;
   a is 1, 2, 3, or 4; and
   R₁′ is hydrogen, —C(O)R′, —OR′, —S(O)_{y}R′, —N(R′)R″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
      R′ and R″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof; and
      y is 0, 1, or 2;
R₂ is —Y₂N(R_{b})R_{c}, wherein:
   Y₂ is alkanediyl_{(C≤8)}, cycloalkanediyl_{(C≤8)}, alkenediyl_{(C≤8)}, alkynediyl_{(C≤8)}, arenediyl_{(C≤8)}, heterocycloalkanediyl_{(C≤8)}, heteroarenediyl_{(C≤8)}, or a substituted version thereof;
   R_{b} and R_{c} are each independently hydrogen, alkyl_{(C≤12)}, cycloalkyl_{(C≤12)}, aryl_{(C≤12)}, heteroaryl_{(C≤12)}, heterocycloalkyl_{(C≤12)}, acyl_{(C≤12)}, or a substituted version thereof; or
R₂ is —Y₃R_{d}, wherein:
   Y₃ is heterocycloalkanediyl_{(C≤8)}, heteroarenediyl_{(C≤8)}, or a substituted version thereof; and
   R_{d} is hydrogen, alkyl_{(C≤12)}, alkenyl_{(C≤12)}, alkynyl_{(C≤12)}, aryl_{(C≤12)}, heteroaryl_{(C≤12)}, heterocycloalkyl_{(C≤12)}, or a substituted version thereof;
      or a pharmaceutically acceptable salt thereof.

In other embodiments, the compounds are further defined as:

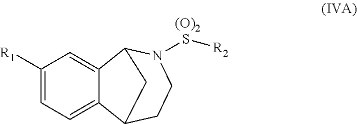

wherein:
R₁ is —Z₁(R₁′)ₐ, wherein:
   Z₁ is arenediyl_{(C≤18)}, substituted arenediyl_{(C≤18)}, heteroarenediyl_{(C≤18)}, substituted heteroarenediyl_{(C≤18)}, when a is 1; arenetriyl_{(C≤18)}, substituted arenetriyl_{(C≤18)}, heteroarenetriyl_{(C≤18)}, substituted heteroarenetriyl_{(C≤18)}, when a is 2; arenequadyl_{(C≤18)}, substituted arenequadyl_{(C≤18)}, heteroarenequadyl_{(C≤18)}, substituted heteroarenequadyl_{(C≤18)}, when a is 3; or arenepentayl_{(C≤18)}, substituted arenepentayl_{(C≤18)}, heteroarenepentayl_{(C≤18)}, substituted heteroarenepentayl_{(C≤18)}, when a is 4;
   a is 1, 2, 3, or 4; and
   R₁′ is hydrogen, —C(O)R′, —OR′, —S(O)_{y}R′, —N(R′)R″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
      R′ and R″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof; and
      y is 0, 1, or 2;
R₂ is —Y₂N(R_{b})R_{c}, wherein:
   Y₂ is alkanediyl_{(C≤8)}, cycloalkanediyl_{(C≤8)}, alkenediyl_{(C≤8)}, alkynediyl_{(C≤8)}, arenediyl_{(C≤8)}, heterocycloalkanediyl_{(C≤8)}, heteroarenediyl_{(C≤8)}, or a substituted version thereof;
   R_{b} and R_{c} are each independently hydrogen, alkyl_{(C≤12)}, cycloalkyl_{(C≤12)}, aryl_{(C≤12)}, heteroaryl_{(C≤12)}, heterocycloalkyl_{(C≤12)}, acyl_{(C≤12)}, or a substituted version thereof; or
R₂ is —Y₃R_{d}, wherein:
   Y₃ is heterocycloalkanediyl_{(C≤8)}, heteroarenediyl_{(C≤8)}, or a substituted version thereof; and
   R_{d} is hydrogen, alkyl_{(C≤12)}, alkenyl_{(C≤12)}, alkynyl_{(C≤12)}, aryl_{(C≤12)}, heteroaryl_{(C≤12)}, heterocycloalkyl_{(C≤12)}, or a substituted version thereof;
      or a pharmaceutically acceptable salt thereof.

In other embodiments, the compounds are further defined as:

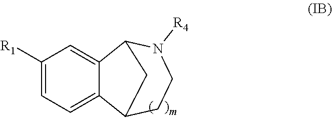

wherein:
m is 1 or 2;
R₃ is —Y₄(Rₑ)_{b}, wherein:
   Y₄ is arenediyl_{(C≤18)}, substituted arenediyl_{(C≤18)}, heteroarenediyl_{(C≤18)}, substituted heteroarenediyl_{(C≤18)}, wherein b is 1; arenetriyl_{(C≤18)}, substituted arenetriyl_{(C≤18)}, heteroarenetriyl_{(C≤18)}, substituted heteroarenetriyl_{(C≤18)}, wherein b is 2; arenequadyl_{(C≤18)}, substituted arenequadyl_{(C≤18)}, heteroarenequadyl_{(C≤18)}, substituted heteroarenequadyl_{(C≤18)}, wherein b is 3; or arenepentayl_{(C≤18)}, substituted arenepentayl_{(C≤18)}, heteroarenepentayl_{(C≤18)}, substituted heteroarenepentayl_{(C≤18)}, wherein b is 4;
   b is 1, 2, 3, or 4; and
   Rₑ is hydrogen, —C(O)Rₑ′, —ORₑ′, —S(O)_{y}Rₑ′, —N(Rₑ′)Rₑ″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
      y is 0, 1, or 2; and
      Rₑ′ and Rₑ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
R₄ is —Y₅N(R_{f})R_{g}, wherein:
   Y₅ is cycloalkanediyl_{(C≤12)}, heterocycloalkyl_{(C≤12)}, or a substituted version of either group;
   R_{f} and R_{g} are each independently hydrogen, alkyl_{(C≤12)}, cycloalkyl_{(C≤12)}, aryl_{(C≤12)}, heteroaryl_{(C≤12)}, heterocycloalkyl_{(C≤12)}, acyl_{(C≤12)}, or a substituted version thereof; or
R₄ is —Y₆R₇, wherein:
   Y₆ is alkanediyl_{(C≤12)} or a substituted version thereof; and
   R₇ is —C(O)Rₕ′, —S(O)_{z}Rₕ′, —N(Rₕ′)Rₕ″, heterocycloalkyl_{(C≤12)}, or substituted heterocycloalkyl_{(C≤12)}; wherein:
      z is 0, 1, or 2; and
      Rₕ′ and Rₕ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
         or a pharmaceutically acceptable salt thereof.

In some embodiments, the compounds are further defined as:

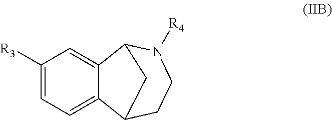

wherein:
R₃ is —Y₄(R_{c})_{b}, wherein:
   Y₄ is arenediyl_{(C≤18)}, substituted arenediyl_{(C≤18)}, heteroarenediyl_{(C≤18)}, substituted heteroarenediyl_{(C≤18)}, wherein b is 1; arenetriyl_{(C≤18)}, substituted arenetriyl_{(C≤18)}, heteroarenetriyl_{(C≤18)}, substituted heteroarenetriyl_{(C≤18)}, wherein b is 2; arenequadyl_{(C≤18)}, substituted arenequadyl_{(C≤18)}, heteroarenequadyl_{(C≤18)}, substituted heteroarenequadyl_{(C≤18)}, wherein b is 3; or arenepentayl_{(C≤18)}, substituted arenepentayl_{(C≤18)}, heteroarenepentayl_{(C≤18)}, substituted heteroarenepentayl_{(C≤18)}, wherein b is 4;
   b is 1, 2, 3, or 4; and
   Rₑ is hydrogen, —C(O)Rₑ′, —ORₑ′, —S(O)_{y}Rₑ′, —N(Rₑ′)Rₑ″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
      y is 0, 1, or 2; and
      Rₑ′ and Rₑ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
R₄ is —Y₆R₇, wherein:
   Y₆ is alkanediyl_{(C≤12)} or a substituted version thereof; and
   R₇ is —C(O)Rₕ′, —S(O)_{z}Rₕ′, —N(Rₕ′)Rₕ″, heterocycloalkyl_{(C≤12)}, or substituted heterocycloalkyl_{(C≤12)}; wherein:
      z is 0, 1, or 2; and
      Rₕ′ and Rₕ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
         or a pharmaceutically acceptable salt thereof.

In some embodiments, the compounds are further defined as:

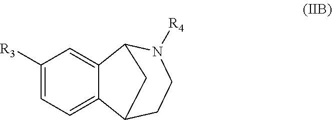

wherein:
R₃ is —Y₄(Rₑ)_{b}, wherein:
   Y₄ is arenediyl_{(C≤18)}, substituted arenediyl_{(C≤18)}, heteroarenediyl_{(C≤18)}, substituted heteroarenediyl_{(C≤18)}, wherein b is 1; arenetriyl_{(C≤18)}, substituted arenetriyl_{(C≤18)}, heteroarenetriyl_{(C≤18)}, substituted heteroarenetriyl_{(C≤18)}, wherein b is 2; arenequadyl_{(C≤18)}, substituted arenequadyl_{(C≤18)}, heteroarenequadyl_{(C≤18)}, substituted heteroarenequadyl_{(C≤18)}, wherein b is 3; or arenepentayl_{(C≤18)}, substituted arenepentayl_{(C≤18)}, heteroarenepentayl_{(C≤18)}, substituted heteroarenepentayl_{(C≤18)}, wherein b is 4;
   b is 1, 2, 3, or 4; and
   Rₑ is hydrogen, —C(O)Rₑ′, —ORₑ′, —S(O)_{y}Rₑ′, —N(Rₑ′)Rₑ″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
      y is 0, 1, or 2; and
      Rₑ′ and Rₑ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
R₄ is —Y₅N(R_{f})R_{g}, wherein:
   Y₅ is cycloalkanediyl_{(C≤12)}, heterocycloalkyl_{(C≤12)}, or a substituted version of either group;
   R_{f} and R_{g} are each independently hydrogen, alkyl_{(C≤12)}, cycloalkyl_{(C≤12)}, aryl_{(C≤12)}, heteroaryl_{(C≤12)}, heterocycloalkyl_{(C≤12)}, acyl_{(C≤12)}, or a substituted version thereof; or
      or a pharmaceutically acceptable salt thereof.

In other embodiments, the compounds are further defined as:

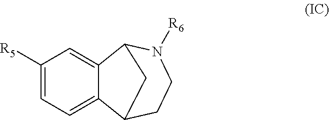

wherein:
R₅ is —Y₇(Rᵢ)_{c}, wherein:
   Y₇ is arenediyl_{(C≤18)}, substituted arenediyl_{(C≤18)}, heteroarenediyl_{(C≤18)}, substituted heteroarenediyl_{(C≤18)}, wherein c is 1, arenetriyl_{(C≤18)}, substituted arenetriyl_{(C≤18)}, heteroarenetriyl_{(C≤18)}, substituted heteroarenetriyl_{(C≤18)}, wherein c is 2; arenequadyl_{(C≤18)}, substituted arenequadyl_{(C≤18)}, heteroarenequadyl_{(C≤18)}, substituted heteroarenequadyl_{(C≤18)}, wherein c is 3; or arenepentayl_{(C≤18)}, substituted arenepentayl_{(C≤18)}, heteroarenepentayl_{(C≤18)}, substituted heteroarenepentayl_{(C≤18)}, wherein c is 4;
   c is 1, 2, 3, or 4; and
   Rᵢ is hydrogen, —C(O)Rᵢ′, —ORᵢ′, —S(O)ᵥRᵢ′, —N(Rᵢ′)Rᵢ″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
      v is 0, 1, or 2; and
      Rᵢ′ and Rᵢ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
R₆ is alkyl_{(C≤12)}, substituted alkyl_{(C≤12)}, cycloalkyl_{(C≤12)}, substituted cycloalkyl_{(C≤12)}, —Y₇R₈, wherein:
   Y₇ is alkanediyl_{(C≤12)} or a substituted version thereof; and
   R₈ is —ORⱼ′, wherein:
      Rⱼ′ is hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
         or a pharmaceutically acceptable salt thereof.

In some embodiments, n is 1. In some embodiments, m is 1. In some embodiments, X₁ is —C(O)Y₁— such as when Y₁ is a covalent bond. In other embodiments, X₁ is —S(O)ₓY₁—. In some embodiments, x is 2. In some embodiments, Y₁ is a covalent bond.

In some embodiments, R₁ is —Z₁(R₁′)ₐ, wherein: Z₁ is arenediyl_{(C≤18)}, substituted arenediyl_{(C≤18)}, heteroarenediyl_{(C≤18)}, substituted heteroarenediyl_{(C≤18)}, when a is 1; arenetriyl_{(C≤18)}, substituted arenetriyl_{(C≤18)}, heteroarenetriyl_{(C≤18)}, substituted heteroarenetriyl_{(C≤18)}, when a is 2; or arenequadyl_{(C≤18)}, substituted arenequadyl_{(C≤18)}, heteroarenequadyl_{(C≤18)}, substituted heteroarenequadyl_{(C≤18)}, when a is 3; a is 1, 2, or 3; and R₁′ is hydrogen, —C(O)R′, —OR′, —S(O)_{y}R′, —N(R′)R″, cyano, halo, alkyl_{(C≤8)}, substituted alkyl_{(C≤8)}, cycloalkyl_{(C≤8)}, cycloalkyl_{(C≤8)}, heterocycloalkyl_{(C≤8)}, or substituted heterocycloalkyl_{(C≤8)}; wherein: R′ and R″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof; and y is 0, 1, or 2.

In some embodiments, a is 1. In some embodiments, Z₁ is arenediyl_{(C≤18)} or substituted arenediyl_{(C≤18)}. In some embodiments, Z₁ is arenediyl_{(C≤18)} such as benzenediyl. In some embodiments, R₁′ is alkyl_{(C≤8)} or substituted alkyl_{(C≤8)}. In some embodiments, R₁′ is substituted alkyl_{(C≤8)} such as trifluoromethyl. In some embodiments, R₁′ is —OR′, wherein R′ is alkyl_{(C≤8)} or substituted alkyl_{(C≤8)}. In some embodiments, R′ is substituted alkyl_{(C≤8)} such as trifluoromethyl.

In other embodiments, a is 2. In some embodiments, Z₁ is arenetriyl_{(C≤18)} or substituted arenetriyl_{(C≤18)}. In some embodiments, Z₁ is arenetriyl_{(C≤18)} such as benzenetriyl. In some embodiments, R₁′ is alkyl_{(C≤8)} or substituted alkyl_{(C≤8)}. In some embodiments, R₁′ is substituted alkyl_{(C≤8)} such as trifluoromethyl. In some embodiments, R₁′ is —OR′, wherein R′ is alkyl_{(C≤8)} or substituted alkyl_{(C≤8)}. In some embodiments, R′ is substituted alkyl_{(C≤8)} such as trifluoromethyl.

In some embodiments, a is 3. In some embodiments, Z₁ is arenequadyl_{(C≤18)} or substituted arenequadyl_{(C≤18)}. In some embodiments, Z₁ is arenequadyl_{(C≤18)} such as benzenequadyl. In some embodiments, R₁′ is alkyl_{(C≤8)} or substituted alkyl_{(C≤8)}. In some embodiments, R₁′ is substituted alkyl_{(C≤8)} such as trifluoromethyl. In some embodiments, R₁′ is —OR′, wherein R′ is alkyl_{(C≤8)} or substituted alkyl_{(C≤8)}. In some embodiments, R′ is substituted alkyl_{(C≤8)} such as trifluoromethyl.

In some embodiments, R₁ is 3-trifluoromethylphenyl, 4-trifluoromethylphenyl, 3-trifluoromethoxyphenyl, or 4-trifluoromethoxyphenyl.

In some embodiments, R₂ is —Y₂N(R_{b})R_{c}. In some embodiments, Y₂ is cycloalkanediyl_{(C≤12)} or substituted cycloalkanediyl_{(C≤12)}. In some embodiments, Y₂ is cycloalkanediyl_{(C≤12)} such as cyclohexanediyl. In some embodiments, R_{b} is hydrogen. In other embodiments, R_{b} is alkyl_{(C≤6)} or substituted alkyl_{(C≤6)}. In some embodiments, R_{b} is alkyl_{(C≤6)} such as methyl. In some embodiments, R_{c} is hydrogen. In some embodiments, R_{c} is alkyl_{(C≤6)} or substituted alkyl_{(C≤6)}. In some embodiments, R_{c} is alkyl_{(C≤6)} such as methyl.

In other embodiments, R₂ is —Y₃R_{d}. In some embodiments, Y₃ is heterocycloalkanediyl_{(C≤12)} or substituted heterocycloalkanediyl_{(C≤12)}. In some embodiments, Y₃ is heterocycloalkanediyl_{(C≤12)} such as 3-aziridinediyl or 4-piperidinediyl. In some embodiments, R_{d} is hydrogen. In other embodiments, R_{d} is alkyl_{(C≤8)} or substituted alkyl_{(C≤8)}. In some embodiments, R_{d} is alkyl_{(C≤8)} such as methyl. In other embodiments, R_{d} is substituted alkyl_{(C≤8)} such as 3-fluoropropyl, 3-hydroxypropyl, 3-methoxypropyl, 2-methoxyethyl, or 2-hydroxyethyl.

In some embodiments, R₃ is —Y₄(Rₑ)_{b}, wherein: Y₄ is arenediyl_{(C≤12)}, substituted arenediyl_{(C≤12)}, arenetriyl_{(C≤18)}, substituted arenetriyl_{(C≤18)}, arenequadyl_{(C≤18)}, or substituted arenequadyl_{(C≤18)}. In some embodiments, b is 2. In some embodiments, Y₄ is arenediyl_{(C≤12)} or substituted arenediyl_{(C≤12)}. In some embodiments, Y₄ is arenediyl_{(C≤12)} such as benzenediyl. In some embodiments, Rₑ is alkyl_{(C≤6)} or substituted alkyl_{(C≤6)}. In some embodiments, Rₑ is substituted alkyl_{(C≤6)} such as trifluoromethyl. In other embodiments, Rₑ is —ORₑ′, wherein Rₑ′ is alkyl_{(C≤6)} or substituted alkyl_{(C≤6)}. In some embodiments, Rₑ′ is substituted alkyl_{(C≤6)} such as trifluoromethyl.

In other embodiments, b is 3. In some embodiments, Y₄ is arenetriyl_{(C≤12)} or substituted arenetriyl_{(C≤12)}. In some embodiments, Y₄ is arenetriyl_{(C≤12)} such as benzenetriyl. In some embodiments, Rₑ is alkyl_{(C≤6)} or substituted alkyl_{(C≤6)}. In some embodiments, Rₑ is substituted alkyl_{(C≤6)} such as trifluoromethyl. In other embodiments, Rₑ is —ORₑ′, wherein Rₑ′ is alkyl_{(C≤6)} or substituted alkyl_{(C≤6)}. In some embodiments, Rₑ′ is substituted alkyl_{(C≤6)} such as trifluoromethyl.

In other embodiments, b is 4. In some embodiments, Y₄ is arenequadyl_{(C≤12)} or substituted arenequadyl_{(C≤12)}. In some embodiments, Y₄ is arenequadyl_{(C≤12)} such as benzenequadyl. In some embodiments, Rₑ is alkyl_{(C≤6)} or substituted alkyl_{(C≤6)}. In some embodiments, Rₑ is substituted alkyl_{(C≤6)} such as trifluoromethyl. In other embodiments, Rₑ is —ORₑ′, wherein Rₑ′ is alkyl_{(C≤6)} or substituted alkyl_{(C≤6)}. In some embodiments, Rₑ′ is substituted alkyl_{(C≤6)} such as trifluoromethyl.

In some embodiments, R₄ is —Y₅N(R_{f})R_{g}, wherein: Y₅ is cycloalkanediyl_{(C≤12)} or substituted cycloalkanediyl_{(C≤12)}. In some embodiments, Y₅ is cycloalkanediyl_{(C≤12)} such as cyclohexanediyl. In some embodiments, Rr is hydrogen. In other embodiments, R_{f} is alkyl_{(C≤6)} or substituted alkyl_{(C≤6)}. In some embodiments, R_{f} is alkyl_{(C≤6)} such as methyl. In some embodiments, R_{g} is hydrogen. In some embodiments, R_{g} is alkyl_{(C≤6)} or substituted alkyl_{(C≤6)}. In some embodiments, R_{g} is alkyl_{(C≤6)} such as methyl.

In other embodiments, R₄ is —Y₆R₇, wherein: Y₆ is alkanediyl_{(C≤12)} or a substituted version thereof; and R₇ is —N(Rₕ′)Rₕ″, heterocycloalkyl_{(C≤12)}, or substituted heterocycloalkyl_{(C≤12)}; wherein: z is 0, 1, or 2; and Rₕ′ and Rₕ″ are each independently hydrogen, alkyl_{(C≤8)}, or a substituted version thereof.

In some embodiments, R₅ is —Y₇(Rᵢ)_{c}, wherein: Y₇ is arenediyl_{(C≤18)}, substituted arenediyl_{(C≤18)}, heteroarenediyl_{(C≤18)}, substituted heteroarenediyl_{(C≤18)}, wherein c is 1; arenetriyl_{(C≤18)}, substituted arenetriyl_{(C≤18)}, heteroarenetriyl_{(C≤18)}, substituted heteroarenetriyl_{(C≤18)}, wherein c is 2; arenequadyl_{(C≤18)}, substituted arenequadyl_{(C≤18)}, heteroarenequadyl_{(C≤18)}, substituted heteroarenequadyl_{(C≤18)}, wherein c is 3; c is 1, 2, or 3; and Rᵢ is hydrogen, —C(O)Rᵢ′, —ORᵢ′, —S(O)ᵥRᵢ′, —N(Rᵢ′)Rᵢ″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein: v is 0, 1, or 2; and Rᵢ′ and Rᵢ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof.

In some embodiments, c is 1. In some embodiments, Y₇ is arenediyl_{(C≤18)} or substituted arenediyl_{(C≤18)}. In some embodiments, Y₇ is arenediyl_{(C≤18)} such as benzenediyl. In some embodiments, Rᵢ is alkyl_{(C≤6)} or substituted alkyl_{(C≤6)}. In some embodiments, Rᵢ is substituted alkyl_{(C≤6)} such as trifluoromethyl. In other embodiments, Rᵢ is —ORᵢ′, wherein Rᵢ′ is alkyl_{(C≤6)} or substituted alkyl_{(C≤6)}. In some embodiments, Rᵢ′ is substituted alkyl_{(C≤6)} such as trifluoromethyl.

In other embodiments, c is 2. In some embodiments, Y₇ is arenetriyl_{(C≤18)} or substituted arenetriyl_{(C≤18)}. In some embodiments, Y₇ is arenetriyl_{(C≤18)} such as benzenetriyl. In some embodiments, Rᵢ is alkyl_{(C≤6)} or substituted alkyl_{(C≤6)}. In some embodiments, Rᵢ is substituted alkyl_{(C≤6)} such as trifluoromethyl. In other embodiments, Rᵢ is —ORᵢ′, wherein Rᵢ′ is alkyl_{(C≤6)} or substituted alkyl_{(C≤6)}. In some embodiments, Rᵢ′ is substituted alkyl_{(C≤6)} such as trifluoromethyl.

In other embodiments, c is 3. In some embodiments, Y₇ is arenediyl_{(C≤18)} or substituted arenediyl_{(C≤18)}. In some embodiments, Y₇ is arenediyl_{(C≤18)} such as benzenediyl. In some embodiments, Rᵢ is alkyl_{(C≤6)} or substituted alkyl_{(C≤6)}. In some embodiments, Rᵢ is substituted alkyl_{(C≤6)} such as trifluoromethyl. In other embodiments, Rᵢ is —ORᵢ′, wherein Rᵢ′ is alkyl_{(C≤6)} or substituted alkyl_{(C≤6)}. In some embodiments, Rᵢ′ is substituted alkyl_{(C≤6)} such as trifluoromethyl.

In some embodiments, R₆ is —Y₇R₈, wherein: Y₇ is alkanediyl_{(C≤12)} or a substituted version thereof; and R₈ is —ORⱼ′, wherein: Rⱼ′ is hydrogen, alkyl_{(C≤8)}, or a substituted version thereof. In some embodiments, Y₇ is alkanediyl_{(C≤12)} such as propanediyl. In some embodiments, Rⱼ′ is hydrogen.

In some embodiments, the compound is further defined as:

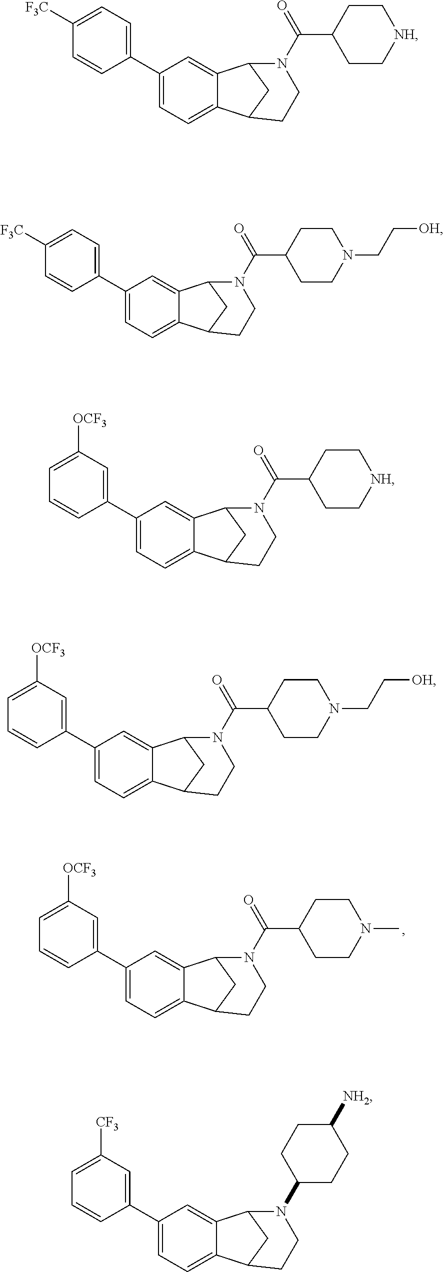

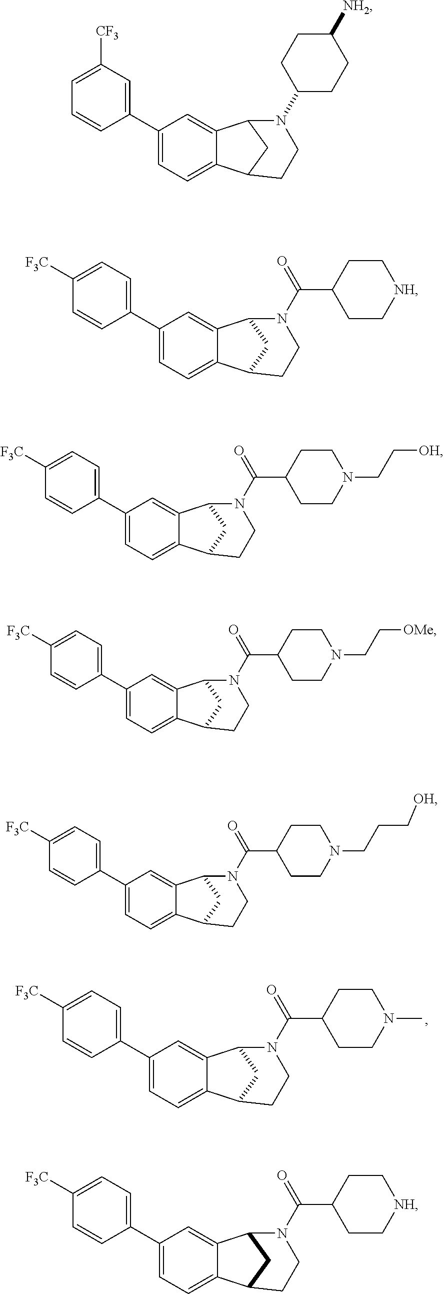

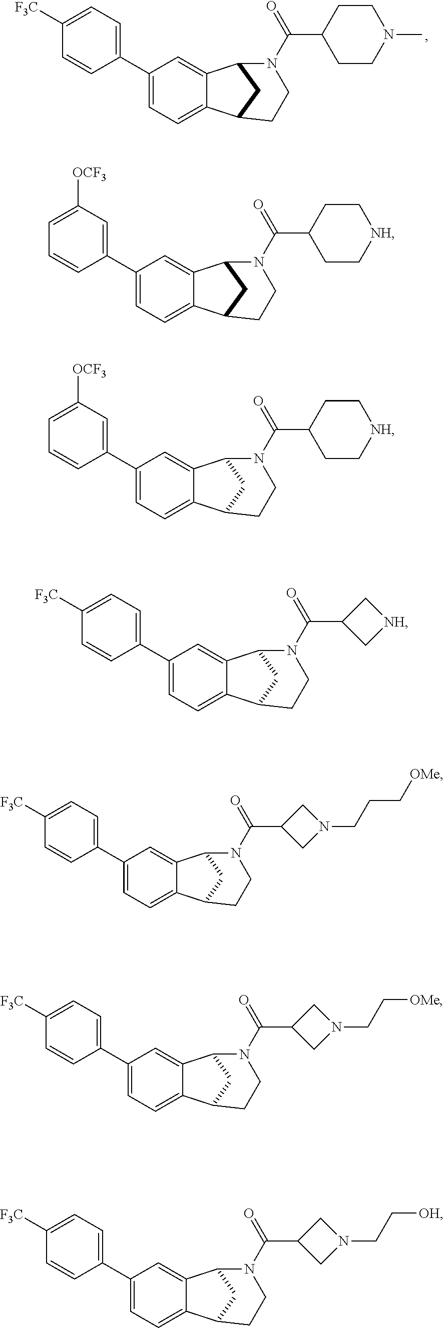

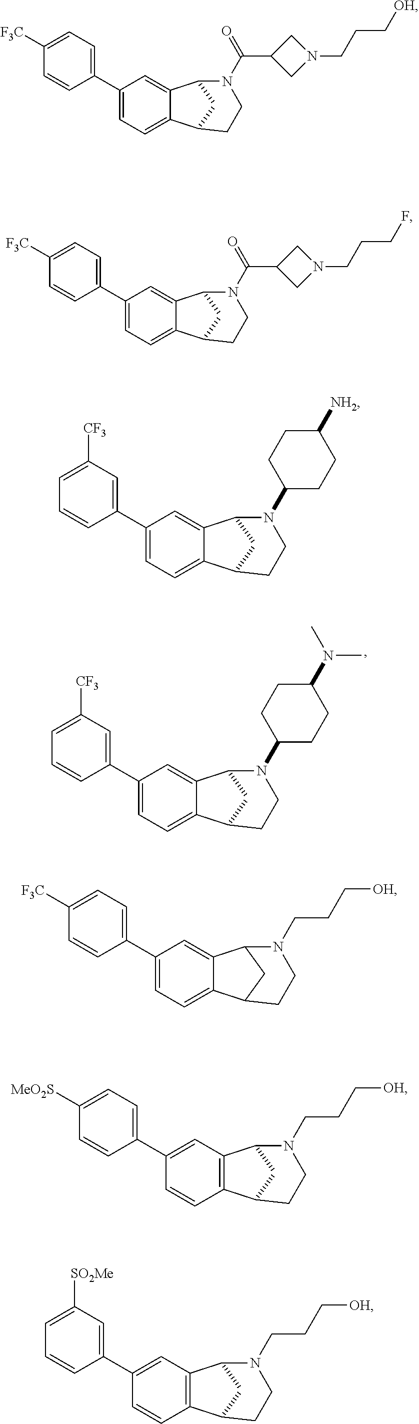

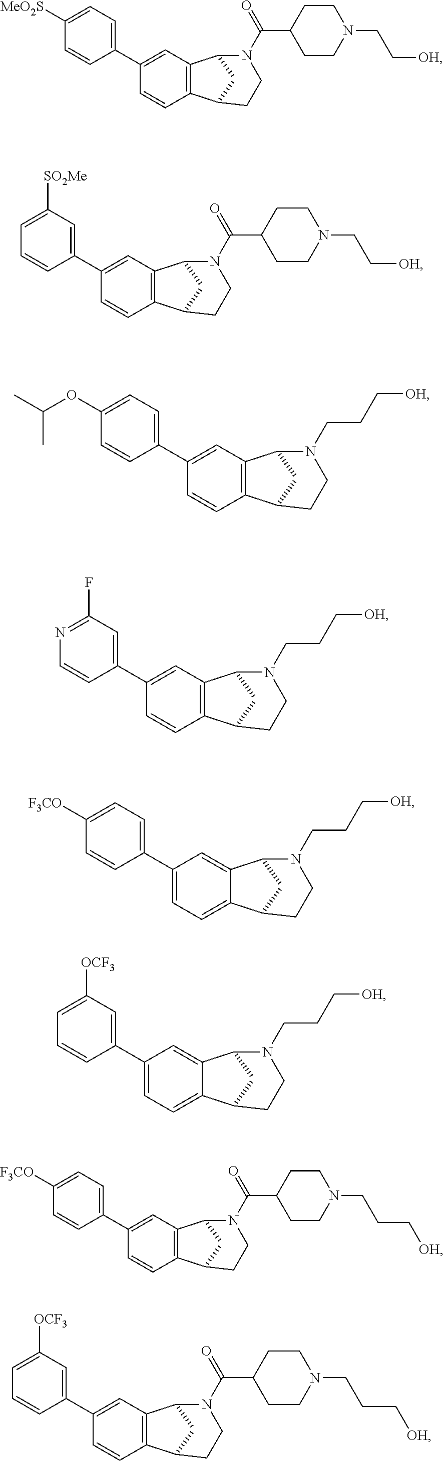

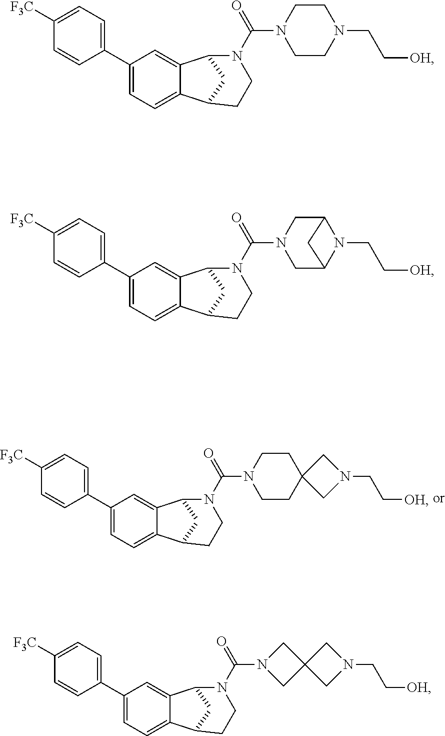

or a pharmaceutically acceptable salt thereof.

In still yet another aspect, the present disclosure provides pharmaceutical compositions comprising:
(A) a compound described herein; and
(B) an excipient.

In some embodiments, the pharmaceutical compositions are formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion.

In some embodiments, the pharmaceutical compositions are formulated for administration via injection. In some embodiments, the pharmaceutical compositions are formulated for oral administration. In some embodiments, the pharmaceutical compositions are formulated as a unit dose.

In still yet another aspect, the present disclosure provides methods of treating a disease or disorder in a patient comprising administering to the patient in need thereof a therapeutically effective amount of a compound or pharmaceutical composition described herein. In some embodiments, the disease or disorder is associated with the misregulation of a sigma 2 or TMEM97 receptor. In some embodiments, the disease or disorder is selected from cancer, neurodegenerative diseases or disorders, withdrawal, anxiety, depression, pain, ophthalmological conditions, or a traumatic brain injury. In some embodiments, the disease or disorder is a neurodegenerative disease or disorder such as Alzheimer's disease, amyotrophic lateral sclerosis, or Huntington's disease. In other embodiments, the disease or disorder is pain. In other embodiments, the disease or disorder is an ophthalmological condition such as retinitis pigmentosa, glaucoma, or dry age-related macular degeneration. In some embodiments, the methods comprise administering the compound or pharmaceutical composition in combination with one or more additional therapeutics. In some embodiments, the methods comprise administering the compound or pharmaceutical composition once. In other embodiments, the methods comprise administering the compound or pharmaceutical composition two or more times.

It is contemplated that any method or composition described herein can be implemented with respect to any other method or composition described herein.

The terms “comprise” (and any form of comprise, such as “comprises” and “comprising”), “have” (and any form of have, such as “has” and “having”), “contain” (and any form of contain, such as “contains” and “containing”), and “include” (and any form of include, such as “includes” and “including”) are open-ended linking verbs. As a result, a method, composition, kit, or system that “comprises,” “has,” “contains,” or “includes” one or more recited steps or elements possesses those recited steps or elements, but is not limited to possessing only those steps or elements; it may possess (i.e., cover) elements or steps that are not recited. Likewise, an element of a method, composition, kit, or system that “comprises,” “has,” “contains,” or “includes” one or more recited features possesses those features, but is not limited to possessing only those features; it may possess features that are not recited.

Any embodiment of any of the present methods, composition, kit, and systems may consist of or consist essentially of—rather than comprise/include/contain/have—the described steps and/or features. Thus, in any of the claims, the term “consisting of” or “consisting essentially of” may be substituted for any of the open-ended linking verbs recited above, in order to change the scope of a given claim from what it would otherwise be using the open-ended linking verb.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features, and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the structures of several σ₂R/TMEM97-selective modulators that were tested for neuroprotective effects in primary cortical neurons against mHTT-induced toxicity.

### DESCRIPTION OF ILLUSTRATIVE EMBODIMENTS

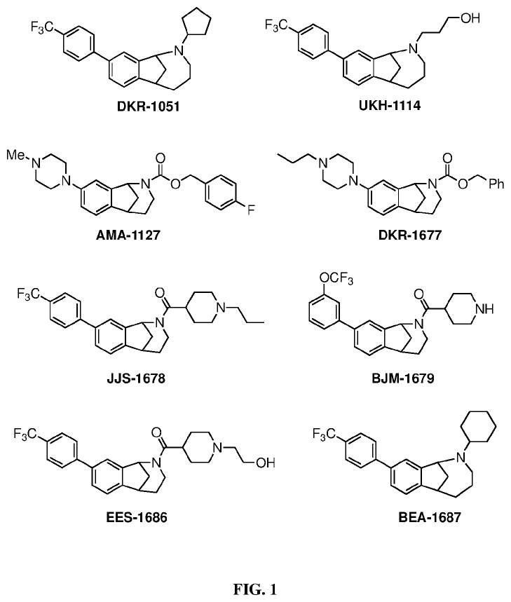

Toward testing the hypothesis that selective modulators of σ₂R/TMEM97 have neuroprotective attributes in Huntington's disease, a small panel of compounds were evaluated with differing affinities and selectivities for σ₂R/TMEM97 in a primary neuron model of HD. These compounds include racemic DKR-1051, UKH-1114, AMA-1127, DKR-1677, JJS-1678, BJM-1679, EES-1686, and BEA-1687 (FIG. 1), some of which have been previously tested in other disease models (Sahn et al, 2017; Vazquez-Rosa et al, 2019; Yi et al, 2017). In the present disclosure, these compounds are tested in an HD cell model to assess the effects of these compounds upon mHTT-induced neuronal toxicity. Briefly, primary neurons were co-transfected with plasmid expression of a 586 N-terminal Htt polypeptide with either normal Q (Htt-N586-22Q) or expanded Q (Htt-N586-82Q) repeats and green fluorescent protein (GFP). Some of these compounds showed strong protective effects on mHTT-induced neuronal cell death. These studies are the first to demonstrate that compounds that bind selectively to σ₂R/TMEM97 are neuroprotective in an HD model, and they support further mechanistic studies of the function of σ₂R/TMEM97 in mHTT protection as a possible new approach to treat HD.

The present disclosure relates to compounds that may be used in the modulation of σ₂R/TMEM97 compared to other σ receptors. These compounds may show one or more improved properties such as increased activity

### A. CHEMICAL DEFINITIONS

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “ [symbol] ” means a single bond, “ [symbol] ” means a double bond, and “ [symbol] ” means triple bond. The symbol “ [symbol] ” represents an optional bond, which if present is either single or double. The symbol “ [symbol] ” represents a single bond or a double bond. Thus, the formula

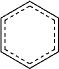

covers, for example,

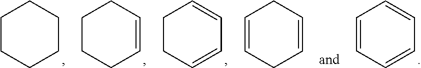

And it is understood that no one such ring atom forms part of more than one double bond. Furthermore, it is noted that the covalent bond symbol “ [symbol] ”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

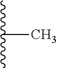

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

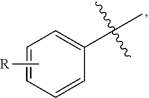

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

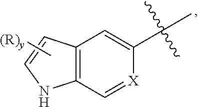

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “alkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C₁₋₄-alkyl”, “C1-4-alkyl”, “alkyl_{(C1-4)}”, and “alkyl_{(C≤4)}” are all synonymous. Except as noted below, every carbon atom is counted to determine whether the group or compound falls with the specified number of carbon atoms. For example, the group dihexylamino is an example of a dialkylamino_{(C12)} group; however, it is not an example of a dialkylamino (co) group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. An aromatic compound or chemical group may be depicted as a single resonance structure; however, depiction of one resonance structure is taken to also refer to any other resonance structure. For example:

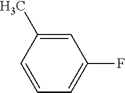

is also taken to refer to

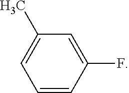

Aromatic compounds may also be depicted using a circle to represent the delocalized nature of the electrons in the fully conjugated cyclic π system, two non-limiting examples of which are shown below:

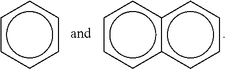

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃) CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. If more than one ring is present, the rings may be fused, bridged, or spirocyclic. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

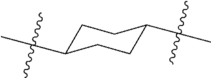 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The terms “arenediyl”, “arenetriyl”, “arenequadyl”, and “arenepentayl” refers to a divalent, trivalent, quadvalent, or pentavalent aromatic group with two, three, four, or five aromatic carbon atoms as points of attachment, said carbon atoms forming part of one or more six-membered aromatic ring structures, each with six ring atoms that are all carbon, and wherein the divalent, trivalent, quadvalent, or pentavalent group consists of no atoms other than carbon and hydrogen. As used herein, the term arenediyl, arenetriyl, arenequadyl, and arenepentayl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl, arenetriyl, arenequadyl, and arenepentayl groups include:

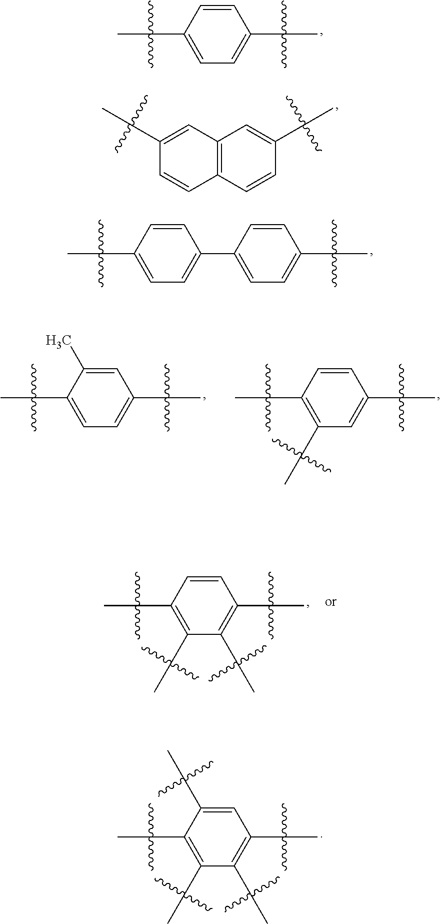

An “arene” refers to the class of compounds having the formula H—R, wherein R is aryl as that term is defined above. Benzene and toluene are non-limiting examples of arenes.

The term “aralkyl” refers to the monovalent group-alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The terms “heteroarenediyl”, “heteroarenetriyl”, “heteroarenequadyl”, and “heteroarenepentayl” refers to a divalent, trivalent, quadvalent, or pentavalent aromatic group, with either an aromatic carbon atom or aromatic nitrogen atom as the points of attachment, said atoms forming part of one or more aromatic ring structures, each with three to eight ring atoms, wherein at least one of the ring atoms of the aromatic ring structure(s) is nitrogen, oxygen or sulfur, and wherein the divalent, trivalent, quadvalent, or pentavalent group consists of no atoms other than carbon, hydrogen, aromatic nitrogen, aromatic oxygen and aromatic sulfur. If more than one ring is present, the rings are fused; however, the terms heteroarenediyl, heteroarenetriyl, heteroarenequadyl, and heteroarenepentayl does not preclude the presence of one or more alkyl or aryl groups (carbon number limitation permitting) attached to one or more ring atoms. Non-limiting examples of heteroarenediyl, heteroarenetriyl, heteroarenequadyl, and heteroarenepentayl groups include:

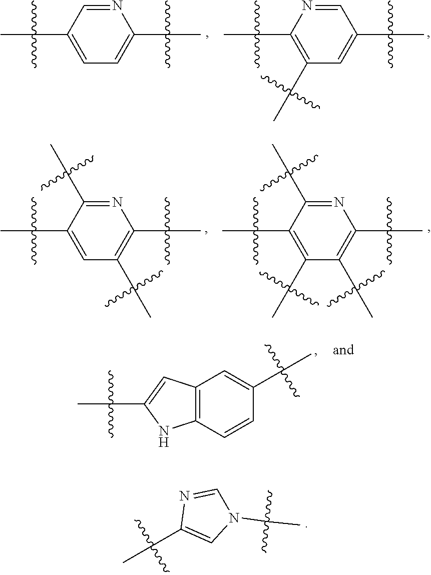 [SMILES: *c1ccc(*)nc1 ; *c1cnc(*)c(*)c1 ; *c1cc(*)c(*)nc1* ; *c1nc(*)c(*)c(*)c1* ; *c1ccc2[nH]c(*)cc2c1 ; *c1cn(*)cn1]

The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes.

The term “heteroaralkyl” refers to the monovalent group -alkanediyl-heteroaryl, in which the terms alkanediyl and heteroaryl are each used in a manner consistent with the definitions provided above. Non-limiting examples are: pyridinylmethyl and 2-quinolinyl-ethyl.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings may be fused, bridged, or spirocyclic. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, tetrahydropyridinyl, pyranyl, oxiranyl, and oxetanyl. The term “N-heterocycloalkyl” refers to a heterocycloalkyl group with a nitrogen atom as the point of attachment. N-pyrrolidinyl is an example of such a group. The term “heterocycloalkanediyl” refers to a divalent cyclic group, with two carbon atoms, two nitrogen atoms, or one carbon atom and one nitrogen atom as the two points of attachment, said atoms forming part of one or more ring structure(s) wherein at least one of the ring atoms of the non-aromatic ring structure(s) is nitrogen, oxygen or sulfur, and wherein the divalent group consists of no atoms other than carbon, hydrogen, nitrogen, oxygen and sulfur. If more than one ring is present, the rings may be fused, bridged, or spirocyclic. As used herein, the term heterocycloalkanediyl does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkanediyl groups include:

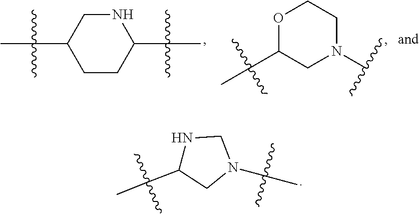 [SMILES: *C1CCC(*)NC1 ; *C1CN(*)CCO1 ; *C1CN(*)CN1]

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O) R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The term “amido” (acylamino), when used without the “substituted” modifier, refers to the group —NHR, in which R is acyl, as that term is defined above. A non-limiting example of an amido group is —NHC(O)CH₃.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active pharmaceutical ingredient of the present invention. The prodrug itself may or may not have activity in its prodrug form. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

In the context of this application, “selectively” means that greater than 50% of the activity of the compound is exhibited in the noted location or the compound is at least twice as active in the desired target. On the other hand, “preferentially” means that greater than 75% of the activity of the compound is exhibited in the noted location or the compound is at least three times as active in the desired target.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2″, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that for tetrahedral stereogenic centers the stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### B. COMPOUNDS OF THE PRESENT DISCLOSURE

The compounds of the present disclosure are shown, for example, above, in the summary of the invention section, and in the claims below. They may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

In some aspects, the present disclosure relates to compounds of the formula:

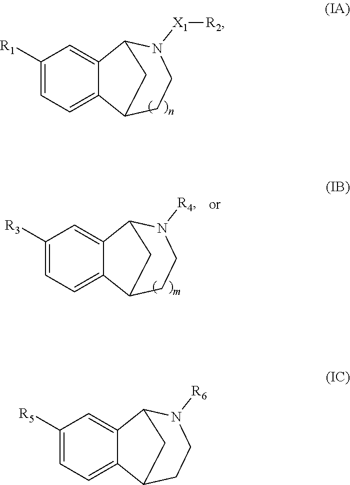

wherein:
m and n are each independently 1 or 2;
R₁ is —Z₁(R₁′)ₐ, wherein:
   Z₁ is arenediyl_{(C≤18)}, heteroarenediyl_{(C≤18)}, or a substituted version thereof, when a is 1, arenetriyl_{(C≤18)}, heteroarenetriyl_{(C≤18)}, or a substituted version thereof, when a is 2, arenequadyl_{(C≤18)}, heteroarenequadyl_{(C≤18)}, or a substituted version thereof, when a is 3, or arenepentayl_{(C≤18)}, heteroarenepentayl_{(C≤18)}, or a substituted version thereof, when a is 4;
   a is 1, 2, 3, or 4; and
   R₁′ is hydrogen, —C(O)R′, —OR′, —S(O)_{y}R′, —N(R′)R″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
      R′ and R″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof; and
      y is 0, 1, or 2;
X₁ is —C(O)Y₁— or —S(O)ₓY₁—, wherein:
   x is 0, 1, or 2; and
   Y₁ is a covalent bond; —O—; —NRₐ—, wherein Rₐ is hydrogen, alkyl_{(C≤6)}, or substituted alkyl_{(C≤6)}; alkanediyl_{(C≤8)}; or substituted alkanediyl_{(C≤8)}; and
R₂ is —Y₂N(R_{b})R_{c}, wherein:
   Y₂ is alkanediyl_{(C≤8)}, cycloalkanediyl_{(C≤8)}, alkenediyl_{(C≤8)}, alkynediyl_{(C≤8)}, arenediyl_{(C≤8)}, heterocycloalkanediyl_{(C≤8)}, heteroarenediyl_{(C≤8)}, or a substituted version thereof;
   R_{b} and R_{c} are each independently hydrogen, alkyl_{(C≤12)}, cycloalkyl_{(C≤12)}, aryl_{(C≤12)}, heteroaryl_{(C≤12)}, heterocycloalkyl_{(C≤12)}, acyl_{(C≤12)}, or a substituted version thereof; or
R₂ is —Y₃R_{d}, wherein:
   Y₃ is heterocycloalkanediyl_{(C≤8)}, heteroarenediyl_{(C≤8)}, or a substituted version thereof; and
   R_{d} is hydrogen, alkyl_{(C≤12)}, alkenyl_{(C≤12)}, alkynyl_{(C≤12)}, aryl_{(C≤12)}, heteroaryl_{(C≤12)}, heterocycloalkyl_{(C≤12)}, or a substituted version thereof;
R₃ is —Y₄(Rₑ)_{b}, wherein:
   Y₄ is arenediyl_{(C≤18)}, heteroarenediyl_{(C≤18)}, or a substituted version thereof, wherein b is 1, arenetriyl_{(C≤18)}, heteroarenetriyl_{(C≤18)}, or a substituted version thereof, wherein b is 2, arenequadyl_{(C≤18)}, heteroarenequadyl_{(C≤18)}, or a substituted version thereof, wherein b is 3, or arenepentayl_{(C≤18)}, heteroarenepentayl_{(C≤18)}, or a substituted version thereof, wherein b is 4;
   b is 1, 2, 3, or 4; and
   Rₑ is hydrogen, —C(O)Rₑ′, —ORₑ′, —S(O)_{y}Rₑ′, —N(Rₑ′)Rₑ″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
      y is 0, 1, or 2; and
      Rₑ′ and Rₑ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
R₄ is —Y₅N(R_{f})R_{g}, wherein:
   Y₅ is cycloalkanediyl_{(C≤12)}, heterocycloalkyl_{(C≤12)}, or a substituted version of either group;
   R_{f} and R_{g} are each independently hydrogen, alkyl_{(C≤12)}, cycloalkyl_{(C≤12)}, aryl_{(C≤12)}, heteroaryl_{(C≤12)}, heterocycloalkyl_{(C≤12)}, acyl_{(C≤12)}, or a substituted version thereof; or
R₄ is —Y₆R₇, wherein:
   Y₆ is alkanediyl_{(C≤12)} or a substituted version thereof; and
   R₇ is —C(O)Rₕ′, —S(O)_{z}Rₕ′, —N(Rₕ′)Rₕ″, heterocycloalkyl_{(C≤12)}, or substituted heterocycloalkyl_{(C≤12)}; wherein:
      z is 0, 1, or 2; and
      Rₕ′ and Rₕ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
R₅ is —Y₇(Rᵢ)_{c}, wherein:
   Y₇ is arenediyl_{(C≤18)}, heteroarenediyl_{(C≤18)}, or a substituted version thereof, wherein c is 1, arenetriyl_{(C≤18)}, heteroarenetriyl_{(C≤18)}, or a substituted version thereof, wherein c is 2, arenequadyl_{(C≤18)}, heteroarenequadyl_{(C≤18)}, or a substituted version thereof, wherein c is 3, or arenepentayl_{(C≤18)}, heteroarenepentayl_{(C≤18)}, or a substituted version thereof, wherein c is 4;
   c is 1, 2, 3, or 4; and
   Rᵢ is hydrogen, —C(O)Rᵢ′, —ORᵢ′, —S(O)ᵥRᵢ′, —N(Rᵢ′)Rᵢ″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
      v is 0, 1, or 2; and
      Rᵢ′ and Rᵢ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
R₆ is —Y₇R₈, wherein:
   Y₇ is alkanediyl_{(C≤12)} or a substituted version thereof; and
   R₈ is —ORⱼ′, wherein:
      Rⱼ′ is hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino (C≤8), dialkylamino_{(C≤8)}, or a substituted version thereof;
or a pharmaceutically acceptable salt thereof.

TABLE 1
Compounds with Binding Values

| Compound | σ2R/TMEM97 Kᵢ (nM) |
|---|---|
| 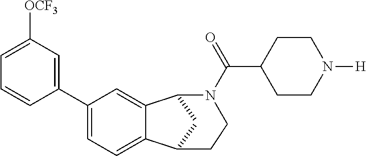 | 35.4 |
| 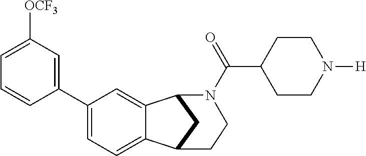 | 37.1 |
| 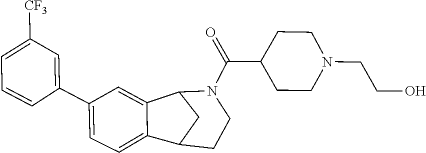 [SMILES: O=C(C1CCN(CCO)CC1)N1CCC2CC1c1cc(-c3cccc(C(F)(F)F)c3)ccc12] | 32.3 |
| 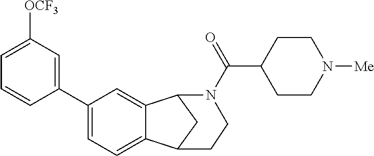 [SMILES: CN1CCC(C(=O)N2CCC3CC2c2cc(-c4cccc(OC(F)(F)F)c4)ccc23)CC1] | 30.4 |
| 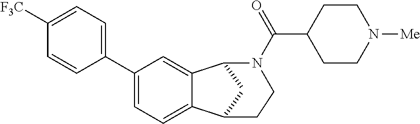 | 32.8 |
| 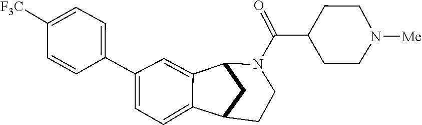 | 35.1 |
| 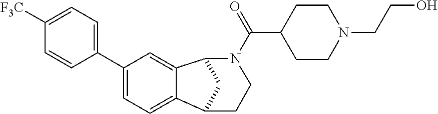 | 13.6 |
| 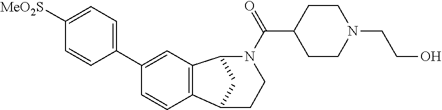 | 834 |
| 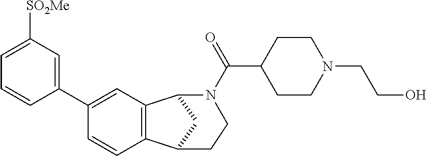 | 277 |
| 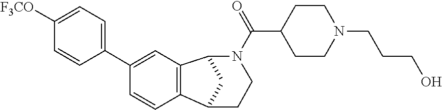 | 79.9 |
| 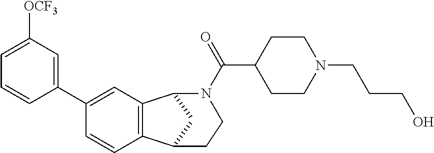 | 264 |
| 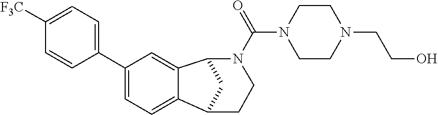 | 955 |
| 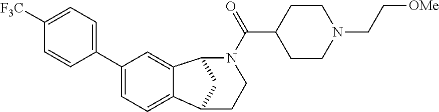 | 10.3 |
| 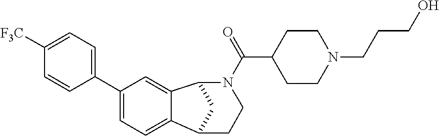 | 7.7 |
| 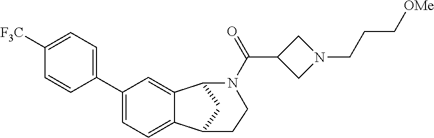 | 4.6 |
| 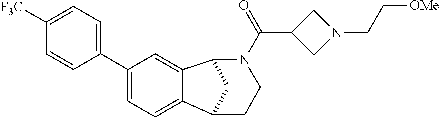 | 17.6 |
| 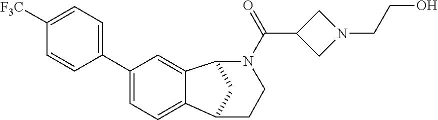 | 15.3 |
| 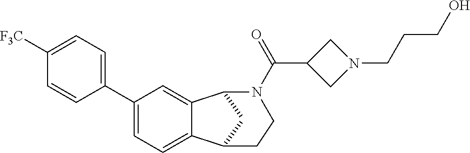 | 11.9 |
| 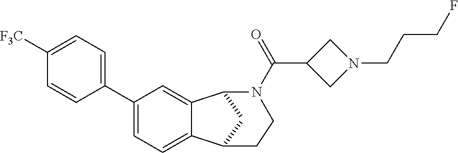 | 14.7 |
| 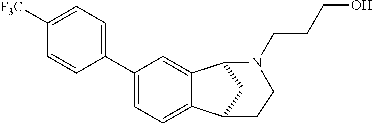 | 30.6 |
| 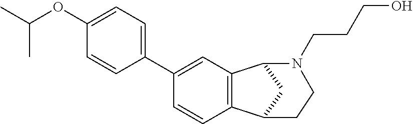 | 65.9 |
| 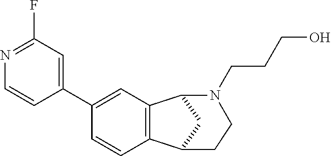 | 802 |
| 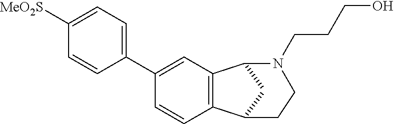 | 1181 |
| 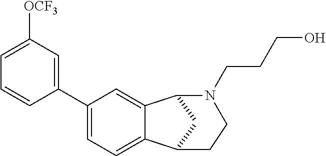 | 48.3 |
| 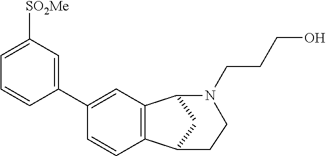 | 1848 |
| 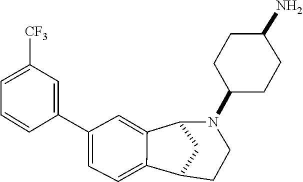 | 30.8 |
| 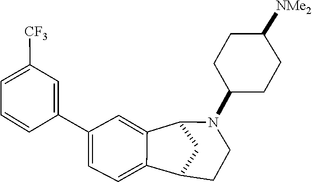 | 9.5 |
| 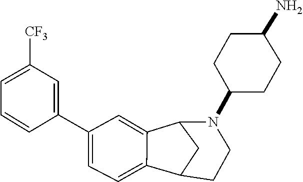 [SMILES: N[C@H]1CC[C@@H](N2CCC3CC2c2cc(-c4cccc(C(F)(F)F)c4)ccc23)CC1] | 7.9 |
| 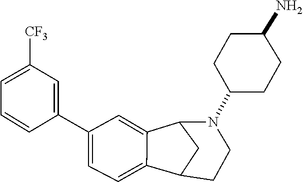 [SMILES: N[C@H]1CC[C@H](N2CCC3CC2c2cc(-c4cccc(C(F)(F)F)c4)ccc23)CC1] | 70.5 |
| 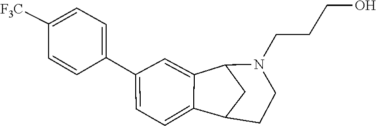 [SMILES: OCCCN1CCC2CC1c1cc(-c3ccc(C(F)(F)F)cc3)ccc12] | 11 |
| 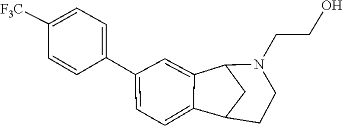 [SMILES: OCCN1CCC2CC1c1cc(-c3ccc(C(F)(F)F)cc3)ccc12] | 32 |
| 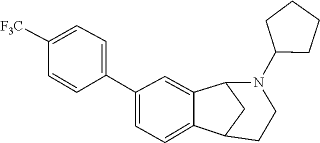 [SMILES: FC(F)(F)c1ccc(-c2ccc3c(c2)C2CC3CCN2C2CCCC2)cc1] | 82.3 |
| 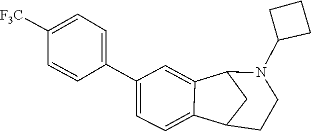 [SMILES: FC(F)(F)c1ccc(-c2ccc3c(c2)C2CC3CCN2C2CCC2)cc1] | 213 |
| 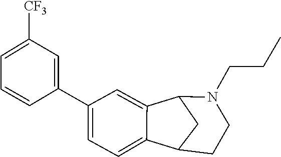 [SMILES: CCCN1CCC2CC1c1cc(-c3cccc(C(F)(F)F)c3)ccc12] | 111.7 |
| 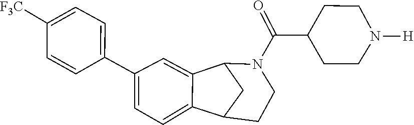 [SMILES: O=C(C1CCNCC1)N1CCC2CC1c1cc(-c3ccc(C(F)(F)F)cc3)ccc12] | 6.8 |
| 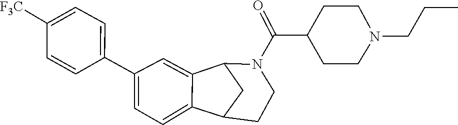 [SMILES: CCCN1CCC(C(=O)N2CCC3CC2c2cc(-c4ccc(C(F)(F)F)cc4)ccc23)CC1] | 1.3 |
| 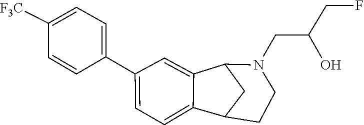 [SMILES: OC(CF)CN1CCC2CC1c1cc(-c3ccc(C(F)(F)F)cc3)ccc12] | 34 |
| 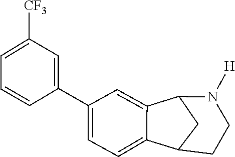 [SMILES: FC(F)(F)c1cccc(-c2ccc3c(c2)C2CC3CCN2)c1] | 57 |
| 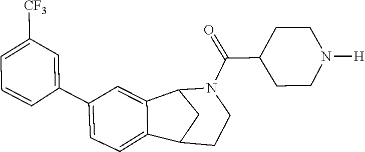 [SMILES: O=C(C1CCNCC1)N1CCC2CC1c1cc(-c3cccc(C(F)(F)F)c3)ccc12] | 5.5 |
| 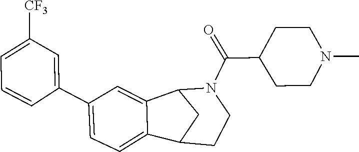 [SMILES: CN1CCC(C(=O)N2CCC3CC2c2cc(-c4cccc(C(F)(F)F)c4)ccc23)CC1] | 2.3 |
| 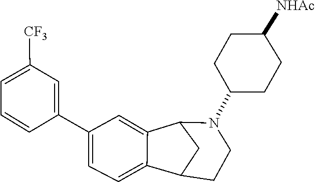 [SMILES: CC(=O)N[C@H]1CC[C@H](N2CCC3CC2c2cc(-c4cccc(C(F)(F)F)c4)ccc23)CC1] | 4.1 |
| 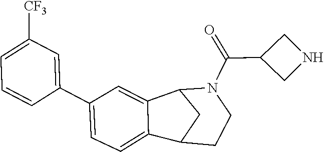 [SMILES: O=C(C1CNC1)N1CCC2CC1c1cc(-c3cccc(C(F)(F)F)c3)ccc12] | 16 |
| 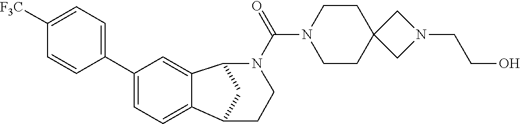 | 91.6 |
| 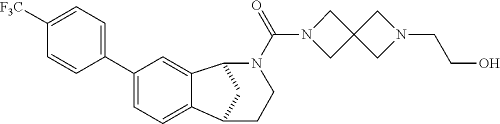 | 441 |
| 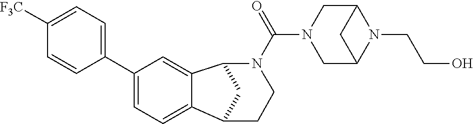 | 62.9 |
| 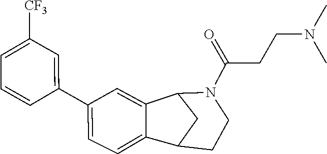 [SMILES: CN(C)CCC(=O)N1CCC2CC1c1cc(-c3cccc(C(F)(F)F)c3)ccc12] | 8.7 |
| 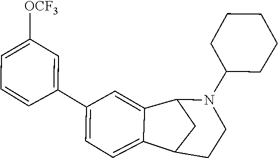 [SMILES: FC(F)(F)Oc1cccc(-c2ccc3c(c2)C2CC3CCN2C2CCCCC2)c1] | 79 |
| 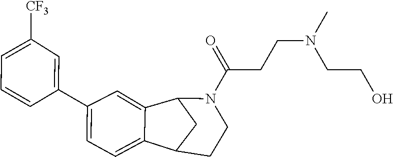 [SMILES: CN(CCO)CCC(=O)N1CCC2CC1c1cc(-c3cccc(C(F)(F)F)c3)ccc12] | 4.3 |
| 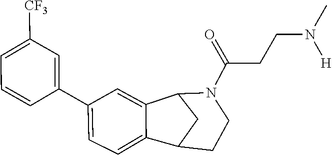 [SMILES: CNCCC(=O)N1CCC2CC1c1cc(-c3cccc(C(F)(F)F)c3)ccc12] | 4.9 |
| 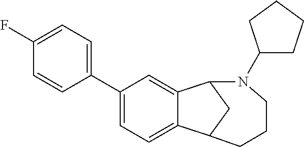 [SMILES: Fc1ccc(-c2ccc3c(c2)C2CC3CCCN2C2CCCC2)cc1] | 152.7 |
| 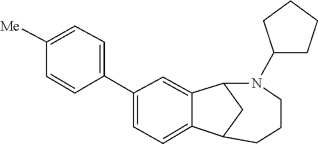 [SMILES: Cc1ccc(-c2ccc3c(c2)C2CC3CCCN2C2CCCC2)cc1] | 187 |
| 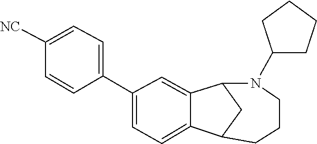 [SMILES: N#Cc1ccc(-c2ccc3c(c2)C2CC3CCCN2C2CCCC2)cc1] | 119 |
| 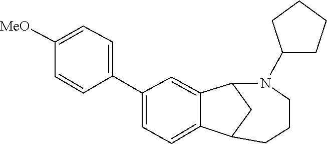 [SMILES: COc1ccc(-c2ccc3c(c2)C2CC3CCCN2C2CCCC2)cc1] | 194 |
| 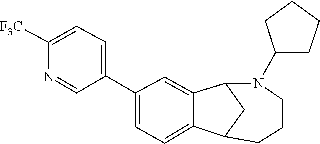 [SMILES: FC(F)(F)c1ccc(-c2ccc3c(c2)C2CC3CCCN2C2CCCC2)cn1] | 336 |
| 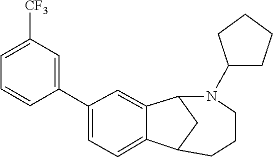 [SMILES: FC(F)(F)c1cccc(-c2ccc3c(c2)C2CC3CCCN2C2CCCC2)c1] | 62 |
| 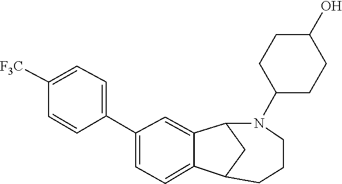 [SMILES: OC1CCC(N2CCCC3CC2c2cc(-c4ccc(C(F)(F)F)cc4)ccc23)CC1] | 39 |
| 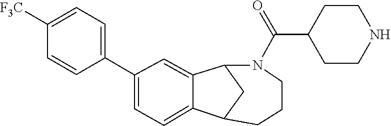 [SMILES: O=C(C1CCNCC1)N1CCCC2CC1c1cc(-c3ccc(C(F)(F)F)cc3)ccc12] | 52.5 |
| 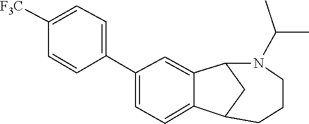 [SMILES: CC(C)N1CCCC2CC1c1cc(-c3ccc(C(F)(F)F)cc3)ccc12] | 87 |
| 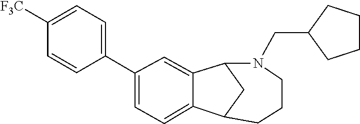 [SMILES: FC(F)(F)c1ccc(-c2ccc3c(c2)C2CC3CCCN2CC2CCCC2)cc1] | 99 |
| 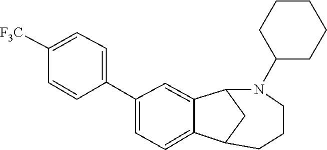 [SMILES: FC(F)(F)c1ccc(-c2ccc3c(c2)C2CC3CCCN2C2CCCCC2)cc1] | 25 |
| 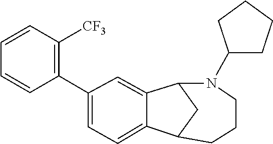 [SMILES: FC(F)(F)c1ccccc1-c1ccc2c(c1)C1CC2CCCN1C1CCCC1] | 549 |
| 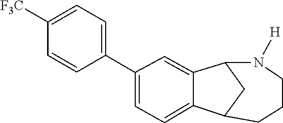 [SMILES: FC(F)(F)c1ccc(-c2ccc3c(c2)C2CC3CCCN2)cc1] | 21 |

All the compounds of the present disclosure may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present invention are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present disclosure have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Compounds of the present disclosure may contain one or more asymmetrically substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration. In some embodiments, the present compounds may contain two or more atoms which have a defined stereochemical orientation.

Chemical formulas used to represent compounds of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, compounds of the present disclosure function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, compounds of the present disclosure exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present invention.

### C. INDICATIONS

In some embodiments of the invention, a disease is treated by targeting the sigma-2 receptor, σ₂R/TMEM9, which is an 18-21 kDa membrane receptor located in lipid rafts that plays a role in hormonal, calcium, and neuronal signaling. The receptor can bind hormones and sterols (e.g. testosterone, progesterone, and cholesterol) and mediate signaling cascades via a calcium secondary messenger. High densities of the receptor can be found in the several areas of the CNS (e.g. cerebellum, motor cortex, hippocampus, substantia nigra, nucleus accumbens, central grey matter, olfactory bulb, subventricular zone, and oculomotor nucleus), liver, and kidney and the receptor is responsible for motor function and emotional response. The receptor is pharmacologically defined as a high-affinity binding site for di-o-tolylguanidine (DTG; Kᵢ=21.2 nM) and haloperidol (Kᵢ=48.7 nM), but with low affinity for (+)-benzomorphans. This contrasts with the sigma-1 receptor, which shows high affinity for all three compounds. The receptor has been shown to bind antipsychotic drugs (e.g. haloperidol, MIN-101), implicating it in a number of neuropsychiatric disorders associated with mood (affect) and emotional responses. Furthermore, the receptor is overexpressed in several cancer cell lines and proliferating tumors, rendering it a key cancer biomarker and potential therapeutic target.

In some embodiments, a disease is treated by targeting σ₂R/TMEM9. σ₂R/TMEM9 is a four pass ER-resident transmembrane protein that has been identified as a modulator of cholesterol levels. H. sapiens TMEM97, also known as MAC30, has the following cDNA and protein sequences:

TMEM97 cDNA sequence (528)
SEQ ID NO: 1

| | |
|---|---|
| 1 | ATGGGGGCTC CGGCAACCAG GCGCTGCGTG GAGTGGCTGC |
| | TGGGCCTCTA CTTCCTCAGC |
| 61 | CACATCCCCA TCACCCTGTT CATGGACCTG CAGGCGGTGC |
| | TGCCGCGCGA GCTCTACCCA |
| 121 | GTCGAGTTTA GAAACCTGCT GAAGTGGTAT GCTAAGGAGT |
| | TCAAAGACCC ACTGCTACAG |
| 181 | GAGCCCCCAG CCTGGTTTAA GTCCTTTCTG TTTTGCGAGC |
| | TTGTGTTTCA GCTGCCTTTC |
| 241 | TTTCCCATTG CAACGTATGC CTTCCTCAAA GGAAGCTGCA |
| | AGTGGATTCG AACTCCTGCA |
| 301 | ATCATCTACT CTGTTCACAC CATGACAACC TTAATTCCGA |
| | TACTCTCCAC ATTTCTGTTT |
| 361 | GAGGATTTCT CCAAAGCCAG TGGTTTCAAG GGACAAAGAC |
| | CTGAGACTTT GCATGAACGG |
| 421 | TTAACCCTTG TGTCTGTCTA TGCCCCCTAC TTACTCATCC |
| | CATTCATACT TTTAATTTTC |
| 481 | ATGTTGCGGA GCCCCTACTA CAAGTATGAA GAGAAAAGAA |
| | AAAAAAAA |

TMEM97 protein sequence (176)
SEQ ID NO: 2
MGAPATRRCVEWLLGLYFLSHIPITLFMDLQAVLPRELYPVEFRNLLKWY
AKEFKDPLLQEPPAWFKSFLFCELVFQLPFFPIATYAFLKGSCKWIRTPA
IIYSVHTMTTLIPILSTFLFEDFSKASGFKGQRPETLHERLTLVSVYAPY
LLIPFILLIFMLRSPYYKYEEKRKKK

i. Neurodegenerative Diseases or Disorders

Neurodegenerative diseases (NDs) include a wide variety of debilitating afflictions of the central and peripheral nervous systems. Most, however, affect the CNS. Some non-limiting examples of neurodegenerative diseases include, but are not limited to, Alzheimer's disease, Pick's disease, senile dementia, Parkinson's disease, multiple sclerosis, multiple system atrophy, dementia with Lewy bodies, Huntingon's disease, progressive supranuclear palsy, Creutzfeldt-Jakob disease, amyotrophic lateral sclerosis, dementia, motor neuron disease, prion disease, Huntington's disease, tauopathies, Chromosome 17 dementias, hereditary neuropathies, and diseases involving cerebellar degeneration. Exemplary neurodegenerative disease could be a condition requiring neuroprotection, stroke, anxiety, depression, Alzheimer's disease, Frontotemporal dementia, Lewy Body dementia, Pick's disease, Huntington's disease, pain, Parkinson's disease, multiple sclerosis, microglia inflammation, schizophrenia, addiction, and head injury (e.g., concussion or traumatic brain injury). Examples of neurological conditions include pain, neuropathic pain, and addiction (e.g., addiction to opioid, cocaine, methamphetamine, and alcohol). Additional neurodegenerative diseases are described in US 2010/0278743, US 2007/0213366, US 2007/0276034, US 2014/0099304, US 2014/0171373, and US 2014/0086880.

a. Alzheimer's Disease

Dementia is a brain disorder that seriously affects a person's ability to carry out daily activities. Alzheimer's disease (AD) is the most common form of dementia among older people. Scientists believe that up to 4 million Americans suffer from AD. The disease usually begins after age 60, and risk goes up with age. While younger people also may get AD, it is much less common. About 3 percent of men and women ages 65 to 74 have AD, and nearly half of those age 85 and older may have the disease.

AD attacks parts of the brain that control thought, memory and language. It was named after Dr. Alois Alzheimer, a German doctor. In 1906, Dr. Alzheimer noticed changes in the brain tissue of a woman who had died of an unusual mental illness. He found abnormal clumps (now called amyloid plaques) and tangled bundles of fibers (now called neurofibrillary tangles). Today, these plaques and tangles in the brain are considered hallmarks of AD.

Scientists also have found other brain changes in people with AD. There is a loss of nerve cells in areas of the brain that are vital to memory and other mental abilities. There also are lower levels of chemicals in the brain that carry complex messages back and forth between nerve cells. Thus, AD may disrupt normal thinking and memory by inhibiting, both physically and chemically, the transfer of message between nerve cells.

AD is progressive, characterized by memory loss, language deterioration, impaired visuospatial skills, poor judgment, indifferent attitude, but preserved motor function. As mentioned, AD usually begins after age 65, but its onset may occur as early as age 40, appearing first as memory decline and, over several years, destroying cognition, personality, and ability to function. Confusion and restlessness may also occur. The type, severity, sequence, and progression of mental changes vary widely. The early symptoms of AD, which include forgetfulness and loss of concentration, can be missed easily because they resemble natural signs of aging. Similar symptoms can also result from fatigue, grief, depression, illness, vision or hearing loss, the use of alcohol or certain medications, or simply the burden of too many details to remember at once.

The course of the disease varies from person to person. Some people have the disease only for the last 5 years of life, while others may have it for as many as 20 years. The most common cause of death in AD patients is infection.

The molecular aspect of AD is complicated and not yet fully defined. AD is characterized by the formation of amyloid plaques and neurofibrillary tangles in the brain, particularly in the hippocampus which is the center for memory processing. Several molecules contribute to these structures: amyloid β protein (Aβ), presenilin (PS), cholesterol, apolipoprotein E (ApoE), and Tau protein. Of these, Aβ appears to play the central role.

Aβ contains approximately 40 amino acid residues. The 42 and 43 residue forms are much more toxic than the 40 residue form. Aβ is generated from an amyloid precursor protein (APP) by sequential proteolysis. One of the enzymes lacks sequence specificity and thus can generate Aβ of varying (39-43) lengths. The toxic forms of Aβ cause abnormal events such as apoptosis, free radical formation, aggregation and inflammation.

Tau protein, associated with microtubules in normal brain, forms paired helical filaments (PHFs) in AD-affected brains which are the primary constituent of neurofibrillary tangles. Recent evidence suggests that Aβ proteins may cause hyperphosphorylation of Tau proteins, leading to disassociation from microtubules and aggregation into PHFs.

b. Huntington's Disease

Huntington's disease (HD) is an autosomal dominant neurodegenerative disease caused by an expanded CAG repeat in the human Huntingtin gene (HTT) that yields an expanded polyglutamine (polyQ) repeat in exon-1 of the human mutant huntingtin (mHTT) protein (The Huntington's Disease Collaborative Research Group, 1993). The mutant huntingtin protein (mHTT) preferentially affects striatum of HD patients, and the disease is manifested as uncontrolled chorea movement, cognitive decline and mood alterations that get progressively worse over time (Walker, 2007). Although some symptomatic treatments are available, there is no disease modifying treatment for HD, so there is an urgent need for neuroprotective therapies (Adam and Jankovic, 2008; Dash and Mestre, 2020).

HD is a devastating disease for which there is neither a cure nor an approved treatment that slows, stops or reverses its progression. HD patients typically develop symptoms at mid-adulthood, and the associated disabilities worsen over time ending in death within 10-20 years following the onset of symptoms (Tabrizi et al, 2020) (Ross, 2004) (Ross and Tabrizi, 2011). Research using cell and animal models has achieved significant progress in understanding the etiology and pathology of HD, but effective treatments have been elusive. HD is caused by mutation in HTT gene that encodes mutant huntingtin (mHTT) with expanded polyglutamine repeats (>37Q) (Ross, 2004; Ross and Tabrizi, 2011). Mutant HTT is specifically toxic to striatal medium spiny neurons and causes neuronal death in the striatum (Tabrizi et al, 2020; Ross, 2004; Ross and Tabrizi, 2011). The mechanism of neuronal death includes mHTT-related transcriptional dysregulation, neurotrophic factor deficit, abnormal mitochondrial function, energy and cholesterol metabolic abnormalities, and impaired protein degradation (Saudou and Humbert, 2016). Although numerous attempts toward discovering agents that can reduce or reverse mHTT toxicity have been unsuccessful, genetic modification of mHTT expression is a promising technique, albeit one limited by central delivery and lack of specificity (Barker et al, 2020; Marxreiter et al, 2020).

c. Neuropathic Pain

“Neuropathic pain” is used according to its plain and ordinary meaning and refers to pain, both episodic and chronic, associated with nerve fiber damage, dysfunction, or injury. Neuropathic pain is a pain initiated or caused by a primary lesion or dysfunction in the nervous system.

For example, neuropathic pain syndromes include postherpetic neuralgia (caused by Herpes Zoster), root avulsions, painful traumatic mononeuropathy, painful polyneuropathy (particularly due to diabetes), central pain syndromes (potentially caused by virtually any lesion at any level of the nervous system), postsurgical pain syndromes (e.g., postmastectomy syndrome, postthoracotomy syndrome, phantom pain), and complex regional pain syndrome (reflex sympathetic dystrophy and causalgia).

In some aspects, the neuropathic pain have typical symptoms like dysesthesias (spontaneous or evoked burning pain, often with a superimposed lancinating component), but pain may also be deep and aching. Other sensations like; hyperesthesia, hyperalgesia, allodynia (pain due to a nonnoxious stimulus), and hyperpathia (particularly unpleasant, exaggerated paid response) may also occur.

Neuropathic pain could be divided into “peripheral” (originating in the peripheral nervous system) and “central” (originating in the brain or spinal cord). Central neuropathic pain is of a type that has a cause that is selected from the following group of causes: cerebral lesions that are predominantly thalamic; infarction, e.g. thalamic infarction or brain stem infarction; cerebral tumors or abscesses compressing the thalamus or brain stem; multiple sclerosis; brain operations, e.g. thalamotomy in cases of motoric disorders; spinal cord lesions; spinal cord injuries; spinal cord operations, e.g. anterolateral cordotomy; ischemic lesions; anterior spinal artery syndrome; Wallenberg's syndrome; and syringomyelia.

In one aspect, the neuropathic pain is a central neuropathic pain syndrome. In some examples the central neuropathic pain syndrome is caused by spinal cord injury and/or spinal cord contusion. In other embodiments, the neuropathic pain is a head pain syndrome caused by central pain mechanisms like in migraine or migraine pain.

Alternatively, the neuropathic pain is a peripheral neuropathic pain. In some examples, the peripheral neuropathic pain is caused by chronic constriction injury or by ligation of the sciatic nerve. The predominantly peripheral neuropathic pain includes a type that is selected from the following types of neuropathic pain and/or has a cause that is selected from the group of the following causes: systemic diseases, e.g. diabetic neuropathy; drug-induced lesions, e.g. neuropathy due to chemotherapy; traumatic syndrome and entrapment syndrome; lesions in nerve roots and posterior ganglia; neuropathies after HIV infections; neuralgia after Herpes infections; nerve roof avulsions; cranial nerve lesions; cranial neuralgia, e.g., trigeminal neuralgia; neuropathic cancer pain; phantom pain; compression of peripheral nerves, neuroplexus and nerve roots; paraneoplastic peripheral neuropathy and ganglionopathy; complications of cancer therapies, e.g. chemotherapy, irradiation, and surgical interventions; complex regional pain syndrome; type I lesions (previously known as sympathetic reflex dystrophy); and type II lesions (corresponding approximately to causalgia). Alternatively, the pain may be a chemically induced pain such as chemotherapy-induced pain is a form of neuropathic pain associated with neurotoxic drugs such as vinca alkaloids.

d. Traumatic Brain Injury

Similarly, the term “traumatic brain injury” or “TBI” is used according to its plain and ordinary meaning and refers to the resultant injury to nerves or the brain caused by an external force. TBI can result in physical, cognitive, social, emotional, and behavioral symptoms and can results in an injury which results in full recovery or permanent disability or damage including death. Even after the initial event, a secondary injury is included in the term traumatic brain injury wherein the cerebral blood flow or pressure within the skulls causes some damage to the brain itself. Additional events which are related to the secondary injury include damage to the blood-brain barrier, release of factors that cause inflammation, free radical overload, excessive release of the neurotransmitter glutamate (excitotoxicity), influx of calcium and sodium ions into neurons, dysfunction of mitochondria, damage to the white matter which results in the separate of cell bodies, changes in the blood flow to the brain; ischemia (insufficient blood flow); cerebral hypoxia (insufficient oxygen in the brain), cerebral edema (swelling of the brain), and raised intracranial pressure (the pressure within the skull). The primary injury results from the initial impact and includes damage from the trauma when tissues and blood vessels are stretched, compressed, and torn.

ii. Hyperproliferative Diseases

While hyperproliferative diseases can be associated with any medical disorder that causes a cell to begin to reproduce uncontrollably, the prototypical example is cancer. One of the key elements of cancer is that the normal apoptotic cycle of the cell is interrupted and thus agents that lead to apoptosis of the cell are important therapeutic agents for treating these diseases. As such, the σ₂R/TMEM9 modulating compounds described in this disclosure may be effective in treating cancers.

Cancer cells that may be treated with the compounds according to the embodiments include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, sarcoma; mesothelioma, malignant; dysgerminoma; embryonal malignant; synovial carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

Therefore, “cancer” refers to all types of cancer, neoplasm, or malignant or benign tumors found in mammals, including leukemia, carcinomas and sarcomas. Exemplary cancers include acute myeloid leukemia (“AML”), chronic myelogenous leukemia (“CML”), and cancer of the brain, breast, triple-negative breast cancer, pancreas, colon, liver, kidney, lung, non-small cell lung, melanoma, ovary, sarcoma, and prostate. Additional examples include, cervix cancers, stomach cancers, head & neck cancers, uterus cancers, mesothelioma, metastatic bone cancer, Medulloblastoma, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, and neoplasms of the endocrine and exocrine pancreas.

a. Leukemia

Leukemia refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The murine leukemia model is widely accepted as being predictive of in vivo anti-leukemic activity. It is believed that a compound that tests positive in the P388 cell assay will generally exhibit some level of anti-leukemic activity regardless of the type of leukemia being treated. Accordingly, the present invention includes a method of treating leukemia, including treating acute myeloid leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

b. Sarcoma

Sarcoma generally refers to a tumor which is made up of a substance like the embryonic connective tissue and is generally composed of closely packed cells embedded in a fibrillar or homogeneous substance. Sarcomas which can be treated with a combination of antineoplastic thiol-binding mitochondrial oxidant and an anticancer agent include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

c. Melanoma

Melanoma is taken to mean a tumor arising from the melanocytic system of the skin and other organs. Melanomas which can be treated with a combination of antineoplastic thiol-binding mitochondrial oxidant and an anticancer agent include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

### D. Carcinoma

Carcinoma refers to a malignant new growth made up of epithelial cells tending to infiltrate the surrounding tissues and give rise to metastases. Exemplary carcinomas which can be treated with a combination of antineoplastic thiol-binding mitochondrial oxidant and an anticancer agent include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

iii. Sterol Homeostasis Diseases

Sterol homeostasis diseases are conditions that disrupt the normal equilibrium of natural steroid alcohols in the cell. These diseases may be caused, for example, by disruption of sterol transport or sterol biogenesis. Exemplary sterol homeostasis diseases are Niemann-Pick disease and Smith-Lemli-Opitz syndrome (SLOS).

a. Niemann-Pick Disease

Niemann-Pick disease is a metabolic disorder in which sphingolipids accumulate in cell lysosomes. Lysosomes are responsible for transportation of material in and out of cells, while mutations that disrupt this process cause the disease. Niemann-Pick disease is commonly divided into four subtypes, type A (NPA), B (NPB), C1 (NPC1), and C2 (NPC2). NPA and NPB are associated with mutations in the SMPD1 gene, a sphingomyelin phosphodisesterase, while mutations in the NPC1 and NPC2 genes are associated with NPC1 and NPC2, respectively. NPC1 and NPC2 function as a tag team of membrane proteins that mediate intracellular cholesterol trafficking in mammals. NPC2 binds cholesterol that has been released in the endosomal lumen and transfers it to the cholesterol-binding pocket of the N-terminal domain of NPC1. NPC1 then exports the cholesterol to the ER and plasma membranes. Thus, loss of or mutations in either of NPC1 or NPC2 perturbs this transportation process and disrupts normal cholesterol homeostasis.

Niemann-Pick disease is inherited and autosomally recessive. Thus, two defective copies of the gene are required for manifestation of the disease. Common symptoms include enlargement of the liver and spleen due to accumulation of sphingomyelin, low platelet count, and persistent lung infection. Furthermore, accumulation of sphingomyelin in the central nervous system (CNS) can result in seizures, ataxia, dysarthria, dysphagia, and a number of other cognitive and physical impairments. NPA is usually childhood lethal by 18 months, NPB presents itself in mid-childhood with survival into adulthood, while NPC1 and NPC2 presents later with some surviving into adulthood. Currently, no effective therapeutics exist for the disease, with most treatments focusing on symptomatic relief.

iv. Ophthalmological Conditions

The compounds of the present disclosure may be used in a variety of different ophthalmological conditions. These conditions may include age-related macular degeneration, cataract, diabetic retinopathy, glaucoma, or retinitis pigmentosa.

a. Age Related Macular Degeneration

Age related macular degeneration (AMD) is a progressive eye condition affecting as many as 10 million Americans. AMD is the number one cause of vision loss and legal blindness in adults over 60 in the U.S. As the population ages, and the “baby boomers” advance into their 60's and 70's, a virtual epidemic of AMD will be prevalent. The disease affects the macula of the eye, where the sharpest central vision occurs. Although it rarely results in complete blindness, it robs the individual of all but the outermost, peripheral vision, leaving only dim images or black holes at the center of vision.

Macular degeneration is categorized as either dry (atrophic) or wet (neovascular). The dry form is more common than the wet, with about 90% of AMD patients diagnosed with dry AMD. The wet form of the disease usually leads to more serious vision loss.

In the dry form, there is a breakdown or thinning of the retinal pigment epithelial cells (RPE) in the macula, hence the term “atrophy”. These RPE cells are important to the function of the retina, as they metabolically support the overlying photoreceptors.

The clinical hallmark of atrophic AMD is accumulation of macular drusen, yellowish deposits just deep to the retinal pigment epithelium (“RPE”). Histopathologic examination of eyes with atrophic AMD reveals deposition of lipid and proteinaceous material deep to the RPE in Bruch's membrane. In aged eyes with AMD, Bruch's membrane is often about 3 times thicker than normal. This thickening is thought to be comprised of lipid as well as modified and cross-linked protein, which impedes transport of nutrients across Bruch's membrane from the choriocapillaris to the outer retina. This thickened barrier comprised of lipid and cross-linked protein impedes transport of nutrients across Bruch's membrane from the choriocapillaris to the outer retina. At present, there is no proven effective treatment for dry AMD other than the use of multivitamins and micronutrients.

Wet AMD occurs when new vessels form and grow through Bruch's membrane into the sub-RPE and subretinal space. This neovascular tissue is very fragile and hyperpermeable. Frequently, it bleeds causing damage to the overlying retina. As the blood organizes, functional macular tissue is replaced by scar tissue. To prevent visual loss, it would be desirable to intervene therapeutically prior to the development of neovascularization.

Although the exact etiology of AMD is not known, several risk factors seem to be important. For example, ARMD may be caused by chronic exposure of the retina to light. The presence or absence of certain nutrients in the diet, such as the antioxidant vitamins E and C, also may affect one's predisposition for ARMD. Other conditions, such as hypertension and smoking, are also considered to be important risk factors for the development of this disease.

AMD is a challenging disease for both patient and doctor, because there are very few treatment options and, with the exception of anti-oxidants, no proven preventative therapy. While some individuals experience only minor inconvenience from macular degeneration, many others with more severe forms of macular degeneration are incapacitated. Current therapies, including laser photocoagulation, photodynamic therapy, and anti-angiogenic therapeutics have had mixed results, and, in certain instances, have caused deleterious side effects.

b. Retinitis Pigmentosa

One of the most devastating conditions affecting the retinal rods is “retinitis pigmentosa,” an inherited disorder which the rods gradually degenerate where the rods become dysfunctional affecting vision. The chief function of the retina is transduction (conversion) of light into nervous impulses by the rods and the cones. Retinitis pigmentosa is a chronic retinal degeneration where the deterioration is accompanied by abnormal deposits of pigment in the rods of the retina. The disease causes a progressive decrease in peripheral vision which this type of vision is the side vision. Eventually, the person with retinitis pigmentosa can see only straight ahead which the patient experiences a condition known as “tunnel vision”. The retinitis pigmentosa was recognized which the condition of RP (retinitis pigmentosa) was classified midway through the last century. There is little known about the causes of RP, the progression, and the treatment of RP.

Retinitis pigmentosa (RP) is a group of inherited diseases that damage the light-sensitive rods and the cones which make up the outer layers of the retina. Rods provide side (peripheral) and night vision. The rods are affected more than the cones. The cones are concentrated in macula called fovea centralis. The foveal centralis provides color and clear sharp central vision, also, called foveal vision which this vision is necessary in humans for reading, for watching television, for driving, and with activities where visual detail is required. The fovea centralis includes parafovea and perifovea of macular regions. Macula lutea is devoid of blood vessels where the macula lutea receives oxygen and nutrition from choroidal BV, across the Bruch's membrane, and retinal pigment epithelium (RPE).

The prevalence of retinitis pigmentosa (RP) in The United States is about 1 in 4000. The worldwide prevalence of RP is about 1 in 3000 where some estimate the prevalence is 1 in 5000. The carrier state is recognized to be approximately 1 in 100. The present invention described herein can be used with known carriers to prevent the development of retinitis pigmentosa. The highest reported incidence of occurrence for RP is among the Navajo Indians where there is 1 in 1878 and the lowest is in Switzerland (1 in 7000). A multicenter population study of retinitis pigmentosa population is 45 years or older. The study found that 52% had 20/40 or better vision in at least one eye, 25% had 20/200 or worse vision, and 0.5% had no light perception. RP is diagnosed in young adulthood where RP can afflict anywhere from infancy to the mid 30s to 50s. The X-linked RP is expressed only in the male. These X-linked varieties point out those men may be affected to some extent more than women.

Retinitis pigmentosa (RP) is not a single disease, but a collection of genetic eye conditions with symptoms of night blindness, which RP precedes tunnel vision for many years. This is due to progressive retinal dystrophy with rods reduction due to the apoptosis in which the RP can lead to blindness. Many people with RP will not become legally blind until, they are in their fifties where these individuals maintain a quantity of sight through their lives. Others go completely blind from RP where some cases result with blindness in early childhood. Development and progression of RP is different in each case. Retinitis pigmentosa is due to abnormalities of the photoreceptors (mostly rods and maybe some cones) and/or the retinal pigment epithelium (RPE) covering of the retina. RP leads to progressive visual loss. For the first time, the Retinitis pigmentosa afflicted experience defective darkness adaptation or nyctalopia (night blindness). The vision defect is followed by reduction of the peripheral visual field (contributing to the term known as tunnel vision), and the loss of central vision which tunnel vision occurs, later, in the course of the disease.

The initial symptom in RP is night blindness (Nyctalopia), which is a painless and progressive. Nyctalopia is considered a feature of the disease. Patients might struggle with tasks at night or in dark places. There is a problem walking in dim lit rooms (e.g., movie theaters), difficulties driving in low light, sundown, misty and cloudy conditions where the individual needs a prolonged period of time to adapt from light to dark. In the early on, the peripheral vision loss is often asymptomatic. Other characteristic symptoms are the reaction to excessively strong light (dazzlement—temporarily deprivation of the sight). A gradual narrowing of the visual field which the field displays itself in the form of difficulty in perceiving objects situated on either side or stumbling over steps or other low down impediment. Such patients may report running into furniture or door frames. The patients struggle with sports such as tennis, softball, football and basketball where peripheral vision is required. Many patients with RP relate to seeing flashes of light (photopsia). The patients describes seeing small, iridescent, blinking lights similar to the symptoms of an ophthalmic migraine aura. However, in contrast to the patient with an ophthalmic migraine, the photopsia may be continuous rather than periodic. The physician has to rule out phenothiazines/thioridazine toxicity to diagnose retinitis pigmentosa. The course of the illness is gradual and variable which the illness leads to vision disability. The direction and the course of the disease can be monitored with computer-averaged and narrow-band passed filtered responses. These studies show that the patients whom ages range from 6 to 49 lose an average 16% of remaining full-field ERG amplitude per year. Cones and rods appear to be functioning normally for their number with their amounts of remaining visual pigment.

The common findings are: Vision changes where the Snelling visual acuity can vary from 20/20 to light perception. This is usually preserved until late in the disease. Pupil reaction can be normal or lacks a defect. Surprisingly, nearly 50% of adult patients develop posterior sub capsular cataracts. This is a hint that there is oxidative damage due to generation of free radicals by the light in both these conditions. The retinal fundus is unaffected early in the disease. Findings on examination include: Bone spicules—Midperipheral retinal hyper pigmentation in a characteristic pattern; Optic nerve waxy pallor appearance; Atrophy of the retinal pigment epithelium in the mid periphery of the retina with retinal arteriolar attenuation; loss of the foveolar reflex or an abnormal vitreoretinal interface. The patient may develop cystoid macular edema coupled with fast and potentially reversible loss of vision. Retinitis pigmentosa, on occasions, shows Retinitis Punctata Albescens, a variant of RP, present with yellow deposits deep in the retina rather than the normal increased pigmentation of the peripheral retina.

Retinitis pigmentosa can be associated with rod-cone retinal degenerations present with central macular pigmentary changes (bull's eye maculopathy), Choroideremia—an X-linked recessive retinal degenerative disease which the disease leads to the degeneration of the choriocapillares, the retinal pigment epithelium and the photoreceptor of the eye. The gyrate atrophy, an autosomal recessive disease, causing progressive chorioretinal degeneration resulting in blindness which the blindness is caused by a deficiency of ornithine-aminotransferase (OAT). This OAT deficiency is described as “atypical retinitis pigmentosa” by Jacobsohn in 1888 which OAT has large characteristic scalloped appearance areas of peripheral retinal atrophy.

c. Glaucoma

Glaucoma is a disease that the ocular pressure elevated due to various etiologies leads to damage and atrophy of the optic nerve, resulting in the abnormal visual field, and thus visual acuity is gradually reduced. Since the optic nerve does not recover once optic nerve atrophy occurs, glaucoma is a refractory disease in that not only vision is lost if glaucoma is untreated, but also the condition is only maintained even after successful treatment, and recovery cannot be expected. Furthermore, ocular hypertension, which may lead to development of glaucoma over a long time although in the absence of visual field defects, also has a similar risk.

Glaucoma is classified into three types: developmental glaucoma, secondary glaucoma, and primary glaucoma. Patients with developmental glaucoma are born with underdevelopment of angle, and obstruction of the aqueous outflow causes this type of glaucoma. Secondary glaucoma arises as a result of clear causes such as inflammation or injury and is caused by ocular co-morbidity such as uveitis or ocular injury as well as hemorrhage due to diabetes, long-term use of steroid hormones for the treatment of other diseases, and the like. Primary glaucoma is a generic name of glaucomas of types with unclear causes and occurs most commonly of glaucomas, with a high incidence among middle aged and elderly persons. Primary glaucoma and secondary glaucoma are further subdivided into two types, open-angle glaucoma and angle closure glaucoma, depending on the blockage of the aqueous outflow. While many patients develop normal tension glaucoma in the absence of elevated intraocular pressure, the primary aim of glaucoma treatment is to lower the intraocular pressure.

For the treatment of glaucoma, laser treatment (laser trabeculoplasty), surgical therapy (trabeculectomy or trabeculotomy), or the like is performed when intraocular pressure cannot be controlled with a drug or a patient with angle closure glaucoma has an acute glaucoma attack, but drug therapy is used as the first line therapy.

Drugs used in the drug therapy of glaucoma include sympathetic nerve stimulants (nonselective stimulants such as epinephrine and α2 stimulants such as apraclonidine), sympathetic nerve blockers (β blockers such as timolol, befunolol, carteolol, nipradilol, betaxolol, levobunolol, and metipranolol and al blockers such as bunazosin hydrochloride), parasympathetic nerve agonists (pilocarpine, etc.), carbonic anhydrase inhibitors (acetazolamide, etc.), prostaglandins (isopropyl unoprostone, latanoprost, travoprost, bimatoprost, tafluprost, etc.), and the like.

Of these drugs, a prostaglandin is a type of drug which facilitates the aqueous outflow from the uveoscleral outflow to lower intraocular pressure and is also commonly used in clinical practice.

Meanwhile, Rho kinase inhibitors have been found as candidate remedies for glaucoma based on a novel mechanism of action. Rho kinase inhibitors lower intraocular pressure by promoting aqueous outflow from the trabecular outflow pathway, and it is further suggested that this action may be attributed to changes in the cytoskeleton of a trabecular cell.

In the treatment of glaucoma and ocular hypertension, drugs having an intraocular pressure lowering action are used in combination to enhance the intraocular pressure lowering action. For example, combination use of a prostaglandin and a sympathetic nerve blocker, glaucoma therapy through ocular administration of a combination of some drugs having an intraocular pressure lowering action, and the like have been reported. Furthermore, a remedy for glaucoma comprising a Rho kinase inhibitor and a prostaglandin in combination have also been reported.

### D. Pharmaceutical Formulations and Routes of Administration

For administration to a mammal in need of such treatment, the σ₂R/TMEM97 modulating compounds of the present disclosure are ordinarily combined with one or more excipients appropriate to the indicated route of administration. The σ₂R/TMEM97 modulating compounds of the present disclosure may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol, and tableted or encapsulated for convenient administration. Alternatively, the σ₂R/TMEM97 modulating compounds of the present disclosure may be dissolved in water, polyethylene glycol, propylene glycol, ethanol, corn oil, cottonseed oil, peanut oil, sesame oil, benzyl alcohol, sodium chloride, and/or various buffers. Other excipients and modes of administration are well and widely known in the pharmaceutical art.

The pharmaceutical compositions useful in the present disclosure may be subjected to conventional pharmaceutical operations such as sterilization and/or may contain conventional pharmaceutical carriers and excipients such as preservatives, stabilizers, wetting agents, emulsifiers, buffers, etc.

The σ₂R/TMEM97 modulating compounds of the present disclosure may be administered by a variety of methods, e.g., orally or by injection (e.g. subcutaneous, intravenous, intraperitoneal, etc.). Depending on the route of administration, the conjugates may be coated in a material to protect the compound from the action of acids and other natural conditions which may inactivate the compound. They may also be administered by continuous perfusion/infusion of a disease or wound site.

To administer the therapeutic compound by other than parenteral administration, it may be necessary to coat the σ₂R/TMEM97 modulating compounds of the present disclosure with or co-administer the σ₂R/TMEM97 modulating compounds of the present disclosure with, a material to prevent its inactivation. For example, the therapeutic compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes. Additionally, Trapasol®, Travasol®, cyclodextrin, and other drug carrier molecules may also be used in combination with the σ₂R/TMEM97 modulating compounds of the present disclosure. It is contemplated that the compounds of the present disclosure may be formulated with a cyclodextrin as a drug carrier using an organic solvent such as dimethylaceamide with a polyethylene glycol and a poloxamer composition in an aqueous sugar solution. In some embodiments, the organic solvent is dimethylsulfoxide, dimethylformamide, dimethylacetamide, or other biologically compatible organic solvents. Additionally, the composition may be diluted with a polyethylene glycol polymer such as PEG100, PEG200, PEG250, PEG400, PEG500, PEG600, PEG750, PEG800, PEG900, PEG1000, PEG2000, PEG2500, PEG3000, or PEG4000. Additionally, the composition may further comprise one or more poloxamer composition wherein the poloxamer contains two hydrophilic polyoxyethylene groups and a hydrophobic polyoxypropylene or a substituted version of these groups. This mixture may be further diluted using an aqueous sugar solution such as 5% aqueous dextrose solution.

The σ2R/TMEM97 modulating compounds of the present disclosure may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion are also envisioned. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating σ2R/TMEM97 modulating compounds of the present disclosure in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (i.e., the therapeutic compound) plus any additional desired ingredient from a previously sterile-filtered solution thereof.

The σ2R/TMEM97 modulating compounds of the present disclosure can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the σ2R/TMEM97 modulating compounds of the present disclosure in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of the σ2R/TMEM97 modulating compounds of the present disclosure calculated to produce the desired therapeutic effect in association with the required pharmaceutical carrier. The specification for the dosage unit forms of the invention are dictated by and directly dependent on (a) the unique characteristics of the σ2R/TMEM97 modulating compounds of the present disclosure and the particular therapeutic effect to be achieved, and (b) the limitations inherent in the art of compounding such a therapeutic compound for the treatment of a selected condition in a patient.

The therapeutic compound may also be administered topically to the skin, eye, or mucosa. Alternatively, if local delivery to the lungs is desired the therapeutic compound may be administered by inhalation in a dry-powder or aerosol formulation.

The σ2R/TMEM97 modulating compounds of the present disclosure describe in this disclosure are administered at a therapeutically effective dosage sufficient to treat a condition associated with a condition in a patient. For example, the efficacy of the σ2R/TMEM97 modulating compounds of the present disclosure can be evaluated in an animal model system that may be predictive of efficacy in treating the disease in humans, such as the model systems shown in the examples and drawings.

The actual dosage amount of the σ2R/TMEM97 modulating compounds of the present disclosure comprising the compounds of the present disclosure administered to a subject may be determined by physical and physiological factors such as age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22(3):659-661, 2008, which is incorporated herein by reference):

$$HED ( mg / kg ) = Animal dose ( mg / kg ) \times ( Animal {K}_{m} / Human {K}_{m} )$$

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

An effective amount typically will vary from about 1 mg/kg to about 50 mg/kg, in one or more dose administrations daily, for one or several days (depending of course of the mode of administration and the factors discussed above). In some particular embodiments, the amount is less than 5,000 mg per day with a range of 10 mg to 4500 mg per day.

The effective amount may be less than 10 mg/kg/day, less than 50 mg/kg/day, less than 100 mg/kg/day, less than 250 mg/kg/day. It may alternatively be in the range of 1 mg/kg/day to 250 mg/kg/day.

In other non-limiting examples, a dose may also comprise from about 0.1 mg/kg/body weight, about 1 mg/kg/body weight, about 10 g/kg/body weight, about 50 g/kg/body weight, or more per administration, and any range derivable therein. In non-limiting examples of a derivable range from the numbers listed herein, a range of about 1 mg/kg/body weight to about 50 mg/kg/body weight, about 5 g/kg/body weight to about 10 g/kg/body weight, etc., can be administered, based on the numbers described above.

In certain embodiments, a pharmaceutical composition of the present disclosure may comprise, for example, at least about 0.1% of a compound described in the present disclosure. In other embodiments, the compound of the present disclosure may comprise between about 0.25% to about 75% of the weight of the unit, or between about 25% to about 60%, or between about 1% to about 10%, for example, and any range derivable therein.

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. In some embodiments, the agents are administered once a day.

The compounds may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may take orally and that the timing of which is or is not dependent upon food intake. Thus, for example, the agents can be taken every morning and/or every evening, regardless of when the subject has eaten or will eat.

### E. Combination Therapy

In addition to being used as a monotherapy, the σ2R/TMEM97 modulating compounds of the present disclosure may also find use in combination therapies. Effective combination therapy may be achieved with a single composition or pharmacological formulation that includes both agents, or with two distinct compositions or formulations, administered at the same time, wherein one composition includes σ2R/TMEM97 modulating compounds and compositions, and the other includes the second agent(s). The other therapeutic modality may be administered before, concurrently with, or following administration of the σ2R/TMEM97 modulating compounds of the present disclosure. The therapy using the σ2R/TMEM97 modulating compounds of the present disclosure may precede or follow administration of the other agent(s) by intervals ranging from minutes to weeks. In embodiments where the other agent and the compounds or compositions of the present disclosure are administered separately, one would generally ensure that a significant period of time did not expire between the time of each delivery, such that each agent would still be able to exert an advantageously combined effect. In such instances, it is contemplated that one would typically administer the σ2R/TMEM97 modulating compounds of the present disclosure and the other therapeutic agent within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of the σ2R/TMEM97 modulating compounds of the present disclosure or the other agent will be desired. In this regard, various combinations may be employed. By way of illustration, where the compounds of the present disclosure are “A” and the other agent is “B”, the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are likewise contemplated. Non-limiting examples of pharmacological agents that may be used in the present invention include any pharmacological agent known to be of benefit in the treatment of the neurological diseases or disorders or cancer.

### D. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Synthesis of Compounds

### A. Materials and Methods

i. Preparation of Solutions of σ₂R/TMEM97 Modulators.

Stock solutions of σ₂R/TMEM97 modulators were prepared by dissolving the compound in DMSO to a concentration of 10 mM. For the in vitro assays, the stock solution was diluted with culture medium (1:1000) to a working concentration of 10 μM of σ₂R/TMEM97 modulator. Serial dilutions were then performed using culture medium to prepare other concentrations of the modulator. The final DMSO concentration is less than 0.1%. The vehicle group was performed using 0.1% DMSO in culture medium.

ii. Receptor Binding Assays.

Receptor binding assays for compounds determined by LC-MS to be >95% pure were performed by the Psychoactive Drug Screening Program (PDSP) at Chapel Hill, North Carolina (Besnard et al, 2012). Briefly, binding affinities, Kᵢ, for σ₂R/TMEM97 (rat PC12 cells) were determined through competition binding assays using the radioligand [³H]-ditolylguanidine in the presence of (+)-pentazocine to block σ₁R binding sites, whereas binding affinities, Kᵢ, for σ₁R (guinea pig brain) were determined through competition binding assays with [³H]-(+)-pentazocine. Binding affinities, Kᵢ, for σ₂R/TMEM97 (human clone transiently expressed in HEK293 cells) were determined through competition binding assays using the radioligand [³H]-ditolylguanidine in the presence of (+)-pentazocine to block σ₁R binding sites, and binding affinities, Kᵢ, for σ₁R (human clone transiently expressed in HEK293 cells) were determined through competition binding assays with [³H]-(+)-pentazocine. Kᵢ values are calculated from best-fit IC₅₀ determinations performed in triplicate. For those σ₂R/TMEM97 modulators that were found to be neuroprotective in these studies, their Kᵢ values for other CNS proteins were also determined by the PDSP. Detailed experimental protocols are available on the NIMH PDSP website at pdspdb.unc.edu/pdspWeb.

### B. Results

i. Receptor Binding Profiles

The binding affinities (Kᵢ) of all synthetic compounds for σ₂R/TMEM97 and σ₁R were determined at the Psychoactive Drug Screening Program. Prior to the identification of σ₂R as TMEM97, Kᵢ values were measured using σ₂R sourced from rat PC12 cells and σ₁R sourced from guinea pig brain, but subsequently σ₂R/TMEM97 and σ₁R binding isotherms were determined using human protein obtained by transfection in HEK293T cells. Examination of the Kᵢ values for AMA-1127, DKR-1051, DKR-1677 and UKH-1114, which were obtained using rat σ₂R/TMEM97 and guinea pig σ₁R proteins, show that each of these compounds has high affinity and good selectivity for σ₂R/TMEM97 vs σ₁R (FIG. 1A). Similarly, the Kᵢ values for JJS-1678, BJM-1679, EES-1686 and BEA-1687, which were obtained using human σ₂R/TMEM97 and σ₁R proteins, also display high affinity and good for σ₂R/TMEM97 vs σ₁R (FIG. 1B). Examination of the structures of these σ₂R/TMEM97-selective compounds reveals that they belong to structural classes similarly to previously identified (Sahn et al, 2016) (Sahn and Martin, 2012) (Sahn et al, 2017). Although the two molecular scaffolds differ by the presence of an extra methylene group in the methanobenzazocines DKR-1051 and UKH-1114, two distinct chemotypes are also represented. Namely, AMA-1127 and DKR-1677 have a basic piperazine group on the aromatic ring of the B-norbenzomorphan core, whereas all of the other compounds have an aryl substituent on the parent molecular framework.

ii. Present Compounds Binding to σ₂R/TMEM97 Protect Cortical Primary Neurons from mHTT Induced Toxicity.

To assess the possible neuroprotective effects of selective σ₂R/TMEM97 modulators in neurodegenerative processes associated with HD, a cell model was used that has been previously described (Arbez et al, 2017). Briefly, primary cortical neurons were transfected with plasmids expressing mHTT (Htt-N586-82Q) and treated with σ₂R/TMEM97 modulators DKR-1051, UKH-1114, AMA-1127, BJM-1679, EES-1686 and BEA-1687 (FIG. 1) at concentrations varying from 0.01-10 μM. These compounds provide significant neuroprotection in a dose dependent fashion at concentrations that range from a low of 10 nM for EES-1686 to a high of 5 μM for BJM-1679. None of these compounds appear to exhibit any intrinsic toxicity (Jin et al., 2023).

### Present Compounds Binding to σ₂R/TMEM97 are Efficacious in Pain Models.

Compounds that bind to σ₂R/TMEM97 have been shown to be efficacious in alleviating neuropathic pain (Sahn et al., 2017). In these studies, earlier generation compounds, DKR-1051 and UKH-1114, relieved pain in the spared nerve injury (SNI) model (Sahn et al, 2017). Among the current compounds, FEM-1689 also reverses mechanical hypersensitivity in wild-type male and female mice after SNI, but it fails to produce an analgesic effect in a global σ₂R/TMEM97 knockout mouse (Yousuf et al, 2023), indicating that the effect was mediated by σ₂R/TMEM97. Moreover, FEM-1689 inhibits the integrated stress response and promotes neurite outgrowth.

### Example 2: Synthesis of Compounds

Chemical Synthesis and Characterization. Acetonitrile was dried by filtration through two columns of activated molecular sieves, and toluene was dried by filtration through one column of activated, neutral alumina followed by one column of Q5 reactant. Methylene chloride and diisopropylethylamine (Hünigs base) were distilled from calcium hydride immediately prior to use. Dioxane was distilled from sodium metal and benzophenone prior to use. All solvents were determined to have less than 50 ppm H₂O by Karl Fischer coulometric moisture analysis. All reagents were reagent grade and used without purification unless otherwise noted. All reactions involving air or moisture sensitive reagents or intermediates were performed under an inert atmosphere of nitrogen or argon in glassware that was flame or oven dried. Solutions were degassed using three freeze-thaw cycles under vacuum. Reaction temperatures refer to the temperature of the cooling/heating bath. Volatile solvents were removed under reduced pressure using a Büchi rotary evaporator at 25-30° C. Thin layer chromatography was performed using run on pre-coated plates of silica gel with a 0.25 mm thickness containing 60F-254 indicator (Merck). Chromatography was performed using forced flow (flash chromatography) and the indicated solvent system on 230-400 mesh silica gel (E. Merck reagent silica gel 60). Radial Preparative Liquid Chromatography (radial plc) was performed on a Chromatotron® using glass plates coated with Merck, TLC grade 7749 silica gel with gypsum binder and fluorescent indicator. All compounds submitted for in vivo testing were >95% purity as determined by LC via AUC at 214- and 254 nm. Proton nuclear magnetic resonance (¹H NMR) and carbon nuclear magnetic resonance (¹³C NMR) spectra were obtained at the indicated field as solutions in CDCl₃ unless otherwise indicated. Chemical shifts are referenced to the deuterated solvent and are reported in parts per million (ppm, δ) downfield from tetramethylsilane (TMS, δ=0.00 ppm). Coupling constants (J) are reported in Hz and the splitting abbreviations used are: s, singlet; d, doublet; t, triplet; q, quartet; m, multiplet; comp, overlapping multiplets of magnetically nonequivalent protons; br, broad; app, apparent. New compounds were prepared according to the reactions summarized in Schemes 1-4.

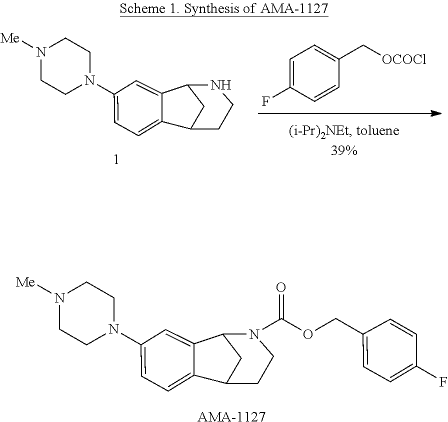

(±)-4-Fluorobenzyl-8-(4-methylpiperazin-1-yl)-1,3,4,5-tetrahydro-2H-1,5-methanobenzo[c]azepine-2-carboxylate (AMA-1127). 4-Fluorobenzyl chloroformate was prepared by slowly adding a solution of phosgene (111 μL of 15 wt % in toluene, 0.155 mmol) to a stirred solution of 4-fluorobenzyl alcohol (21 mg, 0.163 mmol) and diisopropylethylamine (30 mg, 41 μL, 0.233 mmol) in toluene (1 mL) at 0° C. A solution of amine 1 (19 mg, 0.075 mmol) in toluene (0.5 mL) was then added with stirring, the cooling bath was removed, and the solution was stirred for 1 h. The mixture was diluted with aqueous NaOH (1 M, 10 mL), and the aqueous mixture was extracted with EtOAc (3×10 mL). The combined organic extracts were washed with brine (1×10 mL), dried (Na₂SO₄), and concentrated under reduced pressure. The residue was purified via flash column chromatography (SiO₂), eluting with MeOH/CH₂Cl₂ (2% v/v), to afford 12 mg (39%) of AMA-1127 as a pale yellow oil. ¹H NMR (500 MHz) (rotamers) δ 7.46-7.28 (comp, 2H), 7.11 (d, J=8.1 Hz, 1H), 7.10-7.00 (comp, 2H), 6.97 (brs, 0.5H), 6.81 (dd, J=8.1, 2.0 Hz, 1H), 6.77 (brs, 0.5H), 5.45 (dd, J=2.9 Hz, 0.5H), 5.31 (dd, J=2.9 Hz, 0.5H), 5.23-5.02 (comp, 2H), 3.85-3.70 (m, 1H), 3.25-3.10 (comp, 5H), 2.60 (t, J=4.9 Hz, 4H), 2.52-2.38 (m, 1H), 2.37 (s, 3H), 2.25-2.12 (m, 1H), 2.03-1.89 (m, 1H), 1.87-1.80 (m, 1H), 1.62-1.50 (m, 1H). ¹³C NMR (125 MHz) (rotamers) δ 155.1, 154.8, 151.2, 142.3, 141.9, 137.7, 133.2, 132.9, 130.1, 130.1, 130.0, 129.9, 123.3, 123.2, 116.3, 115.9, 115.6, 115.4, 112.6, 112.2, 66.4, 58.2, 57.9, 55.3, 49.8, 46.2, 43.9, 39.1, 38.8, 30.6; HRMS (ESI) m/z calcd for C₂₄H₂₈N₃O₂F (M+H)⁺, 410.2238. found 410.2242.

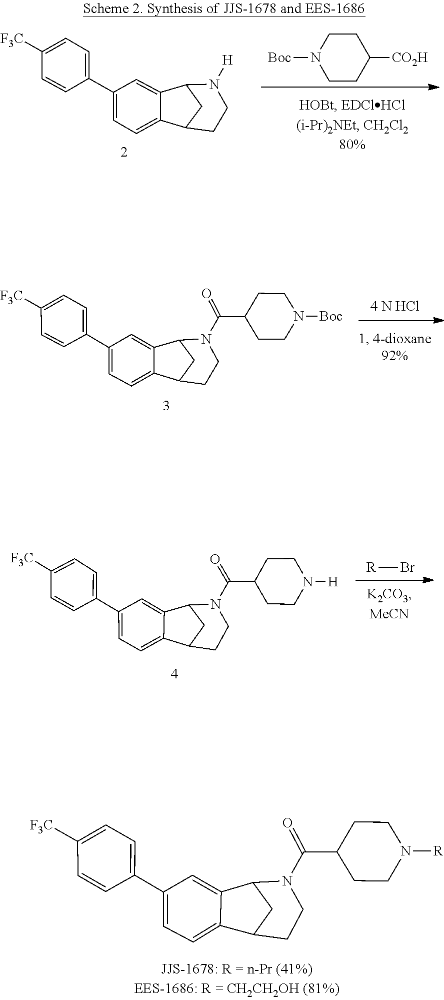

(±)-tert-Butyl 4-(8-(4-(trifluoromethyl)phenyl)-2,3,4,5-tetrahydro-1H-1,5-methanobenzo[c]azepine-2-carbonyl)piperidine-1-carboxylate (3). EDCI·HCl (52 mg, 0.27 mmol) and hydroxybenzotriazole (42 mg, 0.27 mmol) were added to a solution of 1-(tert-butoxycarbonyl)piperidine-4-carboxylic acid (69 mg, 0.30 mmol) in CH₂Cl₂ (3.0 mL). A solution of the amine 2 (75 mg, 0.24 mmol) and Hünig's base (74 mg, 100 μL, 0.54 mmol) in CH₂Cl₂ (0.95 mL) was then added, and the solution was stirred for 12 h. The mixture was concentrated under reduced pressure, and the crude mixture product was purified via radial preparative layer chromatography, eluting with hexanes→hexanes/EtOAc (9/1→1/3→1/1) to provide 102 mg (80%) of the carbamate 3 as a colorless oil. ¹H NMR (400 MHz) δ 7.51 (d, J=8.0 Hz, 1H), 7.49-7.42 (comp, 3H), 7.42-7.37 (m, 1H), 7.37-7.31 (m, 1H), 7.22-7.16 (m, 1H), 6.01 (d, J=4.0 Hz, 0.6H), 5.20-5.17 (m, 0.4H), 4.32 (dd, J=14.0, 7.0 Hz, 0.4H), 4.28-4.02 (comp, 2H), 3.60 (dd, J=14.0, 7.0 Hz, 0.6H), 3.43-3.36 (m, 1H), 2.94-2.80 (m, 1H), 2.72 (td, J=12.0, 5.3 Hz, 2H), 2.56-2.46 (m, 1H), 2.38-2.19 (m, 1H), 2.07-1.97 (m, 1H), 1.86-1.77 (comp, 2H), 1.75-1.63 (comp, 2H), 1.63-1.52 (comp, 2H), 1.51-1.40 (m, 9H); ¹³C NMR (100 MHz) δ 172.4, 154.7, 149.7, 146.5, 144.4, 142.0, 139.2, 129.3 (q, J_{C-F}=25.5 Hz), 127.5, 127.4, 127.2, 125.7 (q, J_{C-F}=3.0 Hz), 123.4, 123.3, 123.1, 122.8, 122.3, 79.5, 54.6, 44.0, 43.2, 39.8, 38.9, 31.1, 29.7, 28.6, 28.4. HRMS (ESI) m/z calcd for C₂₉H₃₃F₃N₂NaO₃ (M+Na)⁺, 537.2335. found 537.2339.

(±)-Piperidin-4-yl(8-(4-(trifluoromethyl)phenyl)-1,3,4,5-tetrahydro-2H-1,5-methanobenzo[c]azepin-2-yl)methanone (4). A solution of 4 N HCl in 1,4-dioxane (3.5 mL) was added to a solution of carbamate 3 (88 mg, 0.17 mmol) in 3.0 mL 1,4-dioxane at room temperature and stirring was continued for 24 h. The solution was concentrated under reduced pressure at room temperature, and the residue was dissolved in CH₂Cl₂ (5 mL). The mixture was made basic by the addition of aqueous NaOH (1 M, 3.0 mL), the organic layer was separated, and the aqueous mixture was extracted with CH₂Cl₂ (3×5 mL). The combined organic extracts were dried (Na₂SO₄), filtered, and concentrated under reduced pressure. The residue was dissolved in Et₂O, filtered, and concentrated under reduced pressure to give 65 mg (92%) of amine 4 that was of sufficient purity to be used in subsequent reactions. ¹H NMR (400 MHz, CD₃OD) δ 7.78-7.72 (m, 2H), 7.71-7.66 (m, 2H), 7.61-7.59 (m, 1H), 7.59-7.57 (m, 0.5H), 7.55-7.53 (m, 0.5H), 7.40-7.36 (m, 1H), 5.87 (d, J=4.0 Hz, 0.6H), 5.42 (d, J=4.0 Hz, 0.4H), 4.19 (dd, J=12.0, 6.0 Hz, 0.4H), 3.78-3.59 (comp, 1.6H), 3.38-3.33 (m, 1H), 3.29 (pent, J=2.0 Hz, 0.6H), 3.16-3.04 (comp, 1.4H), 3.04-2.94 (m, 1H), 2.79-2.49 (comp, 2.6H), 2.35-2.28 (m, 0.4H), 2.26-2.12 (m, 1H), 2.09-1.95 (m, 1H), 1.92 (d, J=12.0 Hz, 0.4H), 1.85-1.79 (m, 1H), 1.81 (d, J=12.0 Hz, 0.6H), 1.75-1.62 (comp, 3H), 1.62-1.46 (m, 1H); ¹³C NMR (100 MHz) δ 171.1, 170.9, 146.6, 146.4, 144.3, 141.7, 140.7, 139.4, 139.3, 128.3, 127.8, 127.4, 127.3, 125.7 (q, J_{C-F}=3.0 Hz), 123.4, 123.2, 122.8, 122.4, 59.1, 44.4, 43.8, 40.2, 39.7, 39.6, 36.7, 35.1, 31.1, 30.4. HRMS (ESI) m/z calcd for C₂₄H₂₆F₃N₂O (M+H)⁺, 415.1992. found 415.2004.

(±)-(1-Propylpiperidin-4-yl)(8-(4-(trifluoromethyl)phenyl)-1,3,4,5-tetrahydro-2H-1,5-methanobenzo[c]azepin-2-yl)methanone (JJS-1678). 1-Bromopropane (22 mg, 0.18 mmol) was added to a mixture of 4 (25 mg, 0.060 mmol) and K₂CO₃ (34 mg, 0.24 mmol) in CH₃CN (800 μL). The mixture was heated at 45° C. for 20 h, cooled to room temperature, and concentrated under reduced pressure. The crude residue was purified via flash column chromatography (SiO₂), eluting with EtOAc→MeOH/EtOAc (1/19) to give 11 mg (41%) of JJS-1678 as an off white foam. ¹H NMR (400 MHz) δ 7.71-7.62 (comp, 4H), 7.57-7.54 (m, 0.6H), 7.51 (dd, J=8.0, 2.0 Hz, 1H), 7.43-7.40 (m, 0.4H), 7.35 (t, J=4.0 Hz, 1H), 6.02 (J=4.0 Hz, 0.6H), 5.18 (J=4.0 Hz, 0.4H), 4.32 (dd, J=12.0, 4.0 Hz, 0.4H), 3.59 (dd, J=12.0, 4.0 Hz, 0.6H), 3.42-3.35 (m, 1H), 3.13-2.95 (comp, 2H), 2.71 (td, J=26.0, 4.0 Hz, 1H), 2.48-2.11 (comp, 5H), 2.08-1.77 (comp, 6H), 1.73-1.64 (m, 1H), 1.62-1.48 (comp, 2H), 0.95-0.89 (m, 1H), 0.89 (t, J=12.0 Hz, 3H); ¹³C NMR (100 MHz) δ 172.2, 171.9, 146.6, 146.4, 144.4, 141.9, 140.9, 139.3, 139.2, 128.2, 127.7, 127.4, 127.3, 125.7 (q, J_{C-F}=3.0 Hz), 123.4, 123.2, 122.7, 122.3, 59.5, 59.4, 44.4, 43.8, 40.1, 39.8, 39.7, 36.4, 31.1, 30.4, 29.67, 18.4, 18.3, 11.6, 11.5. HRMS (ESI) m/z calcd for C₂₇H₃₂F₃N₂O (M+H)⁺, 457.2461. found 457.2465.

(±)-(1-(2-Hydroxyethyl)piperidin-4-yl)(8-(4-(trifluoromethyl)phenyl)-1,3,4,5-tetrahydro-2H-1,5-methanobenzo[c]azepin-2-yl)methanone (EES-1686). 2-Bromoethanol (15 mg, 0.12 mmol) was added to a mixture of 4 (25 mg, 0.060 mmol) and K₂CO₃ (34 mg, 0.24 mmol) in CH₃CN (800 μL), and then the mixture was heated at 50° C. for 20 h. The mixture was cooled to room temperature and concentrated under reduced pressure, and the crude residue was purified via flash column chromatography (SiO₂), eluting with hexanes→hexanes/EtOAc (1/1)→EtOAc→CH₂Cl₂→MeOH/CH₂Cl₂ (1:9) to give 22 mg (81%) of a EES-1686 as a light yellow oil. ¹H NMR (400 MHz) δ 7.71-7.62 (comp, 4H), 7.55 (br s, 0.6H), 7.52 (dd, J=8.0, 2.0 Hz, 1H), 7.42 (br s, 0.4H), 7.35 (t, J=8.0 Hz, 1H), 6.02 (d, J=4.0 Hz, 0.6H), 5.18 (d, J=4.0 Hz, 0.4H), 4.31 (dd, J=12.0, 8.0 Hz, 0.4H), 3.71-3.61 (comp, 2H), 3.58 (dd, J=12.0, 8.0 Hz, 0.6H), 3.43-3.36 (m, 1H), 3.16-2.98 (comp, 2H), 2.87-2.78 (m, 1H), 2.73 (td, J=26.0, 4.0 Hz, 1H), 2.66 (t, J=4.0 Hz, 0.7H), 2.61 (t, J=4.0 Hz, 1.3H), 2.53-2.19 (comp, 4H), 2.08-1.80 (comp, 5H), 1.78-1.64 (comp, 2H), 0.92-0.78 (m, 1H); ¹³C NMR (100 MHz) δ 171.8, 171.5, 146.6, 146.3, 144.3, 141.7, 140.7, 139.4, 139.3, 128.3, 127.8, 127.4, 127.3, 125.7 (q, J_{C-F}=3.0 Hz), 123.5, 123.3, 122.7, 122.3, 60.7, 59.1, 56.4, 51.6, 44.4, 43.8, 40.3, 39.7, 39.6, 36.5, 31.1, 29.7. HRMS (ESI) m/z calcd for C₂₆H₃₀F₃N₂O₂ (M+H)⁺, 459.2263. found 459.2254.

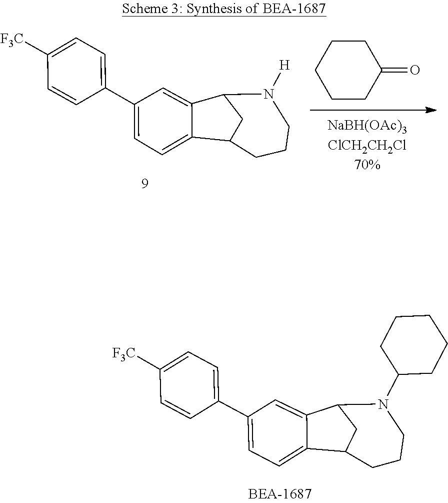

(±)-2-Cyclohexyl-9-(4-(trifluoromethyl)phenyl)-1,2,3,4,5,6-hexahydro-1,6-methanobenzo[c]azocine (BEA-1687). Cyclohexanone (22 mg, 23 μL, 0.22 mmol) was added to a solution of 9 (23 mg, 0.072 mmol) in 1,2-dichloroethane at room temperature, and the solution was stirred for 30 min. Sodium triacetoxyborohydride (47 mg, 0.22 mmol) and acetic acid (100 μL) were added sequentially, and the mixture was stirred 12 h at room temperature. Aqueous saturated NaHCO₃ solution (2 mL) was then added, and the mixture was stirred for 10 min. The layers were separated, and the aqueous layer was extracted with CH₂Cl₂ (3×15 mL). The combined organic layers were washed with brine (3 mL), dried (MgSO₄), filtered and concentrated under reduced pressure. The crude residue was purified via radial preparative layer chromatography, eluting with hexanes hexanes/EtOAc (100/0→95/5) to provide 20 mg (70%) of BEA-1687 as a light yellow oil. ¹H NMR (400 MHz, CD₃OD) δ 7.80 (d, J=12.0 Hz, 2H), 7.71 (d, J=12.0 Hz, 2H), 7.56-7.54 (comp, 1.5H), 7.53 (d, J=4.0 Hz, 0.5H), 7.27 (d, J=8.0 Hz, 1H), 4.43-4.39 (m, 1H), 3.39-3.34 (m, 1H), 3.13 (t, J=12.0 Hz, 1H), 2.65 (m, 1H), 2.60-2.52 (m, 1H), 2.20-2.10 (comp, 3H), 2.00 (br d, J=12.0 Hz, 1H), 1.93-1.85 (m, 2H), 1.82 (pent, J=4.0 Hz, 2H), 1.71-1.64 (m, 1H), 1.64-1.52 (m, 1H), 1.47-1.12 (comp, 5H), 1.07-0.94 (m, 1H). ¹³C NMR (100 MHz) δ 146.6, 143.3, 139.5, 129.7, 129.4, 129.1, 127.9, 125.7 (q, J_{C-F}=3.0 Hz), 125.4, 123.8, 123.2, 66.3, 62.3, 50.5, 42.7, 34.9, 33.8, 29.7, 25.4, 25.3, 25.2. HRMS (ESI) m/z calcd for C₂₅H₂₉F₃N (M+H)⁺, 400.2247. found 400.2253.

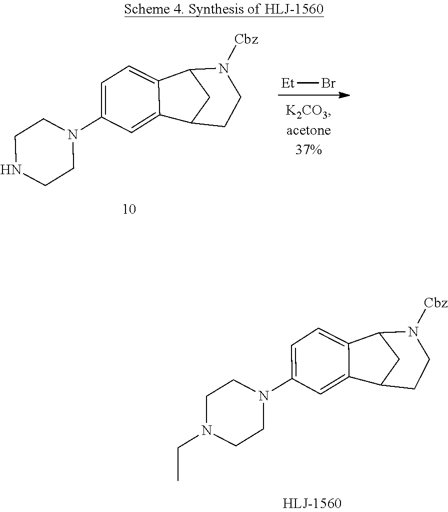

(±)-Benzyl-7-(4-ethylpiperazin-1-yl)-1,3,4,5-tetrahydro-2H-1,5-methanobenzo-[c]azepine-2-carboxylate (HLJ-1560). A mixture of K₂CO₃ (15 mg, 0.106 mmol), 10 (20 mg, 0.053 mmol), and ethyl bromide (7 mg, 4.7 μL, 0.064 mmol) in acetone (1 mL) was stirred at room temperature for 24 h. The mixture was filtered, and the filtrate was concentrated under reduced pressure. The residue was purified via flash column chromatography (SiO₂), eluting with MeOH/CH₂Cl₂ (2% v/v) to afford to afford 8 mg (37%) of HLJ-1560 as a clear oil. ¹H NMR (400 MHz) (rotamers) δ 7.48-7.26 (comp, 5H), 7.21 (d, J=7.8 Hz, 0.5H), 7.10 (d, J=7.5 Hz, 0.5H), 6.84 (d, J=2.2 Hz, 1H), 6.78-6.71 (m, 1H), 5.44 (brs, 0.5H), 5.33 (brs, 0.5H, 5.23-5.05 (comp, 2H), 3.87-3.73 (m, 1H), 3.27-3.17 (comp, 5H), 2.62 (t, J=4.9 Hz, 4H), 2.49 (q, J=7.1 Hz, 2H), 2.51-2.38 (m, 1H), 2.24-2.12 (m, 1H), 2.02-1.80 (comp, 2H), 1.64-1.49 (m, 1H), 1.13 (t, J=7.1 Hz, 3H). ¹³C NMR (125 MHz) (rotamers) δ 155.0, 154.9, 152.1, 147.8, 137.3, 137.1, 132.4, 132.2, 128.6, 128.0, 127.9, 124.5, 124.3, 114.6, 111.0, 67.0, 57.3, 57.1, 53.0, 52.5, 49.6, 44.1, 40.4, 38.7, 30.5, 12.1; IR (neat) 2937, 1695, 1418, 1237, 1096 cm⁻¹; HRMS (ESI) m/z calcd for C₂₅H₃₁N₃O₂ (M+H)⁺, 406.2489. found 406.2503.

### III. Other Compounds

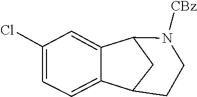

Benzyl 8-chloro-1,3,4,5-tetrahydro-2H-1,5-methanobenzo[c]azepine-2-carboxylate. ¹H NMR (500 MHz, as a mixture of rotamers) δ 7.48-7.27 (comp, 5.5H), 7.24-7.21 (m, 1H), 7.207.12 (comp, 1.5H), 5.50 (br s, 0.5H), 5.38 (br s, 0.5H), 5.25-5.07 (comp, 2H), 3.76-3.91 (m, 1H), 3.29-3.23 (m, 1H), 2.51-2.35 (m, 1H), 2.27-2.13 m, 1H), 2.05-1.93 (m, 1H), 1.87 (d, J=11.0 Hz, 1H), 1.63-1.52 (m, 1H); ¹³C NMR (126 MHz, as a mixture of rotamers) δ 154.9, 154.7, 144.7, 142.9, 142.7, 136.8, 136.7, 132.6, 128.4, 128.0, 127.94, 127.86, 124.2, 124.0, 123.9, 123.8, 67.1, 57.4, 57.2, 43.6, 39.3, 38.5, 30.1. HRMS (ESI) m/z calcd for C₁₉H₁₉NO₂Cl (M+H)⁺ 328.1104. found 328.1106.

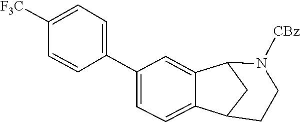

Benzyl 8-(4-(trifluoromethyl)phenyl)-1,3,4,5-tetrahydro-2H-1,5-methanobenzo[c]azepine-2-carboxylate. ¹H NMR (500 MHz, as a mixture of rotamers) δ 7.72-7.24 (comp, 12H), 5.61 (br s, 0.5H), 5.49 (br s, 0.5H), 5.27-5.07 (comp, 2H), 3.96-3.81 (m, 1H), 3.39-3.24 (m, 1H), 2.61-2.43 (m, 1H), 2.34-2.20 (m, 1H), 2.12-2.00 (m, 1H), 1.94 (d, J=11.0 Hz, 1H), 1.74-1.59 (m, 1H). ¹³C NMR (126 MHz, as a mixture of rotamers) δ 155.0, 154.8, 146.5, 144.6, 142.1, 141.9, 139.0, 136.9, 136.8, 129.2 (q, J_{C-F}=31.5 Hz), 128.4, 127.9, 127.8, 127.3, 125.6 (q, J_{C-F}=3.8 Hz), 123.3 (q, J_{C-F}=272.2 Hz), 123.2, 122.7, 122.5, 67.0, 57.6, 57.3, 43.6, 39.5, 38.6, 30.2. HRMS (ESI) m/z calcd for C₂₆H₂₂F₃NNaO₂ (M+Na)⁺, 460.1495. found 460.1499.

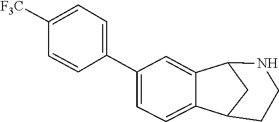 [SMILES: FC(F)(F)c1ccc(-c2ccc3c(c2)C2CC3CCN2)cc1]

8-(4-(Trifluoromethyl)phenyl)-2,3,4,5-tetrahydro-1H-1,5-methanobenzo[c]azepine. ¹H NMR (500 MHz) δ 7.76 (s, 1H), 7.68 (q, J=8.5 Hz, 4H), 7.56 (dd, J=7.5, 1.5 Hz, 1H), 7.34 (d, J=8.0 Hz, 1H), 7.11 (br s, 1H), 4.67 (d, J=2.5 Hz, 1H), 3.38 (s, 1H), 3.07 (dd, J=13.0, 5.5 Hz, 1H), 2.49 (td, J=12.5, 5.0 Hz, 1H), 2.42-2.33 (comp, 2H), 2.22 (td, J=12.5, 5.0 Hz, 1H), 1.67 (d, J=13.5 Hz, 1H). ¹³C NMR (126 MHz) δ 146.1, 144.2, 139.6, 139.1, 129.3 (q, J_{C-F}=32.5 Hz), 128.6, 127.4, 125.7 (q, J_{C-F}=3.8 Hz), 124.2 (q, J_{C-F}=272.2 Hz), 123.2, 123.1, 58.0, 42.7, 38.8, 38.1, 28.3. HRMS (ESI) m/z calcd for C₁₈H₁₇F₃N (M+H)⁺, 304.1308. found 304.1309.

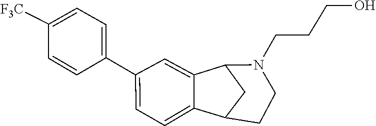 [SMILES: OCCCN1CCC2CC1c1cc(-c3ccc(C(F)(F)F)cc3)ccc12]

3-((1S,5R)-8-(4-(Trifluoromethyl)phenyl)-1,3,4,5-tetrahydro-2H-1,5-methanobenzo[c]azepin-2-yl) propan-1-ol (FEM-1689). ¹H NMR (500 MHz, CDCl₃) δ 7.69 (s, 4H), 7.49 (dd, J=7.6, 1.7 Hz, 1H), 7.39 (s, 1H), 7.31 (d, J=7.6 Hz, 1H), 4.16 (d, J=4.6 Hz, 1H), 3.93-3.79 (comp, 2H), 3.21 (br. s, 1H), 2.84 (dd, J=11.7, 5.7 Hz, 1H), 2.77 (ddd, J=12.0, 7.5, 3.8 Hz, 1H), 2.39 (ddd, J=12.2, 7.8, 3.8 Hz, 1H), 2.32-2.26 (m, 1H), 2.07-1.98 (m, 1H), 1.97 (d, J=11.0 Hz, 1H), 1.86-1.76 (m, 1H), 1.75-1.66 (m, 1H), 1.61-1.55 (m, 1H), 1.50 (td, J=11.9, 4.8 Hz, 1H). ¹³C NMR (126 MHz, CDCl₃) δ 146.9, 145.1, 139.5, 138.4, 129.30 (q, J_{C-F}=32.4 Hz), 127.5, 127.4, 125.8 (q, J_{C-F}=3.8 Hz), 124.6 (q, J_{C-F}=272.4 Hz), 123.2, 123.1, 64.9, 63.6, 56.7, 47.1, 44.7, 39.7, 30.1, 27.2.

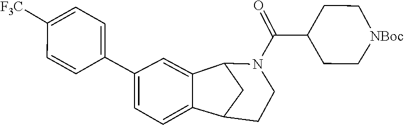 [SMILES: CC(C)(C)OC(=O)N1CCC(C(=O)N2CCC3CC2c2cc(-c4ccc(C(F)(F)F)cc4)ccc23)CC1]

tert-Butyl 4-(8-(4-(trifluoromethyl)phenyl)-2,3,4,5-tetrahydro-1H-1,5-methanobenzo[c]azepine-2-carbonyl)piperidine-1-carboxylate. ¹H NMR (500 MHz, as a mixture of rotamers) δ 7.63-7.28 (comp, 6H), 7.19 (d, J=8.4 Hz, 1H), 6.01 (d, J=4.0 Hz, 0.65H), 5.19 (d, J=4.0 Hz, 0.35H), 4.32 (dd, J=13.6, 6.4 Hz, 0.35H), 3.60 (dd, J=13.6, 6.4 Hz, 0.65H), 3.39 (s, 1H), 2.95-2.61 (comp, 3H), 2.55-2.22 (comp, 2H), 2.04-1.97 (m, 1H), 1.86-1.78 (comp, 2H), 1.74-1.66 (comp, 3H), 1.64-1.50 (comp, 3H), 1.46 (d, J=8.8 Hz, 9H). ¹³C NMR (126 MHz, as a mixture of rotamers) δ 172.4, 171.1, 154.7, 146.7, 144.4, 142.0, 139.2, 129.3 (q, J_{C-F}=32.8 Hz), 127.7, 127.3, 125.7 (q, J_{C-F}=3.8 Hz), 124.2 (q, J_{C-F}=272.8 Hz), 123.2, 122.7, 79.5, 54.6, 44.0, 43.3, 39.8, 38.9, 31.1, 29.7, 28.7, 28.4. HRMS (ESI) m/z calcd for C₂₉H₃₃F₃N₂NaO₃ (M+Na)⁺, 537.2335. found 537.2339.

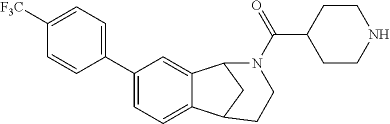 [SMILES: O=C(C1CCNCC1)N1CCC2CC1c1cc(-c3ccc(C(F)(F)F)cc3)ccc12]

Piperidin-4-yl(8-(4-(trifluoromethyl)phenyl)-1,3,4,5-tetrahydro-2H-1,5-methanobenzo[c]azepin-2-yl)methanone. ¹H NMR (400 MHz, as a mixture of rotamers) δ 7.72-7.62 (comp, 4H), 7.56-7.40 (comp, 2H), 7.38-7.31 (m, 1H), 6.00 (d, J=4.0 Hz, 0.65H), 5.19 (d, J=4.0 Hz, 0.35H), 4.30 (dd, J=14.0, 6.4 Hz, 0.35H), 3.37 (s, 1H), 3.27-3.07 (comp, 2H), 2.95-2.51 (comp, 3.65H), 2.25-2.30 (comp, 3H) 2.05-1.96 (m, 1H), 1.86-1.79 (comp, 2H), 1.77-1.66 (comp, 3H). ¹³C NMR (126 MHz, as a mixture of rotamers) δ 171.1, 171.0, 146.6, 146.4, 144.3, 141.7, 140.8, 139.4, 139.3, 129.3 (q, J_{C-F}=32.3 Hz), 128.3, 127.8, 127.4, 127.3, 125.7 (q, J_{C-F}=3.8 Hz), 124.2 (q, J_{C-F}=272.2 Hz), 123.4, 123.2, 122.8, 122.4, 59.1, 54.9, 43.8, 42.8, 42.6, 40.2, 39.6, 35.1, 31.1. HRMS (ESI) m/z calcd for C₂₄H₂₆F₃N₂O (M+H)⁺, 415.1992. found 415.2004.

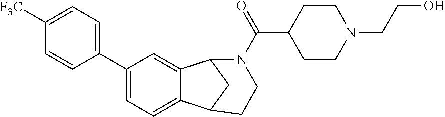 [SMILES: O=C(C1CCN(CCO)CC1)N1CCC2CC1c1cc(-c3ccc(C(F)(F)F)cc3)ccc12]

(1-(2-Hydroxyethyl)piperidin-4-yl)(8-(4-(trifluoromethyl)phenyl)-1,3,4,5-tetrahydro-2H-1,5-methanobenzo[c]azepin-2-yl)methanone. ¹H NMR (500 MHz, as a mixture of rotamers) δ 7.72-7.62 (comp, 4H), 7.56-7.31 (comp, 3H), 5.99 (d, J=4.0 Hz, 0.65H), 5.17 (d, J=4.4 Hz, 0.35H), 4.30 (dd, J=14.0, 6.5 Hz, 0.35H), 3.78-3.68 (comp, 2H), 3.56 (dd, J=14.0, 6.5 Hz, 0.65H), 3.43-3.36 (m, 1H), 3.25-3.07 (comp, 2H), 2.95-2.20 (comp, 7H), 2.14-1.80 (comp, 6H), 1.74-1.66 (m, 1H). ¹³C NMR (126 MHz, as a mixture of rotamers) δ 172.4, 172.1, 146.7, 146.5, 144.4, 142.0, 141.0, 139.3, 139.2, 129.3 (q, J_{C-F}=32.8 Hz), 128.2, 127.6, 127.4, 127.3, 125.7 (q, J_{C-F}=3.8 Hz), 124.2 (q, J_{C-F}=272.2 Hz), 123.4, 123.1, 122.8, 122.3, 59.8, 58.7, 57.3, 57.2, 54.7, 53.4, 52.5, 52.4, 52.3, 44.4, 43.8, 40.0, 39.8, 39.7, 36.4, 31.2, 30.5. HRMS (ESI) m/z calcd for C₂₆H₃₀F₃N₂O₂ (M+H)⁺, 459.2263. found 459.2255.

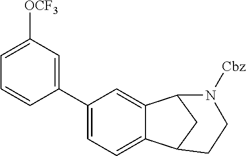 [SMILES: O=C(OCc1ccccc1)N1CCC2CC1c1cc(-c3cccc(OC(F)(F)F)c3)ccc12]

Benzyl 8-(3-(trifluoromethoxy)phenyl)-1,3,4,5-tetrahydro-2H-1,5-methanobenzo[c]azepine-2-carboxylate. ¹H NMR (400 MHz, as a mixture of rotamers) δ 7.52-7.11 (comp, 12H), 5.55 (br s, 0.5H), 5.43 (br s, 0.5H), 5.26-5.02 (comp, 2H), 3.91-3.72 (m, 1H), 3.29-3.16 (m, 1H), 2.56-2.34 (m, 1H), 2.27-2.11 (m, 1H), 2.03-1.91 (m, 1H), 1.87 (d, J=11.2 Hz, 1H), 1.65-1.52 (m, 1H). ¹³C NMR (126 MHz, as a mixture of rotamers) δ 154.9, 149.6 (q, J_{C-F}=2.1 Hz), 146.3, 143.2, 139.0, 130.0, 128.5, 128.0, 127.9, 127.4, 125.4, 123.1, 122.5, 120.5 (q, J_{C-F}=257.0 Hz), 119.7, 119.4, 67.0, 57.4, 43.6, 39.5, 38.6, 30.2. HRMS (ESI) m/z calcd for C₂₆H₂₂F₃NNaO₃ (M+Na)⁺, 476.1444. found 476.1449.

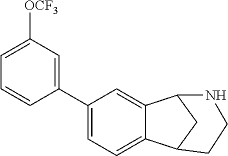 [SMILES: FC(F)(F)Oc1cccc(-c2ccc3c(c2)C2CC3CCN2)c1]

8-(3-(Trifluoromethoxy)phenyl)-2,3,4,5-tetrahydro-1H-1,5-methanobenzo[c]azepine. ¹H NMR (400 MHz) δ 7.55-7.39 (comp, 5H), 7.28 (d, J=7.6 Hz, 1H), 7.20-7.14 (m, 1H), 4.36 (d, J=4.4 Hz, 1H), 3.47-3.39 (m, 1H), 3.29-3.23 (m, 1H), 2.81 (dd, J=12.0, 6.0 Hz, 1H), 2.38 (td, J=12.4, 4.8 Hz, 1H), 2.30-2.22 (m, 1H), 2.08-2.01 (comp, 2H), 1.64-1.53 (m, 1H). ¹³C NMR (100 MHz) δ 149.6 (q, J_{C-F}=2.5 Hz), 146.2, 143.5, 142.9, 138.8, 130.0, 127.0, 125.4, 122.8, 122.0, 120.5 (q, J_{C-F}=322.6 Hz), 119.6, 119.3, 58.7, 44.8, 39.8, 39.0, 30.6. HRMS (ESI) m/z calcd for C₁₈H₁₇F₃NO (M+H)⁺, 320.1257. found 320.1266.

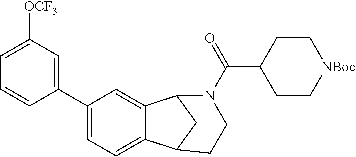 [SMILES: CC(C)(C)OC(=O)N1CCC(C(=O)N2CCC3CC2c2cc(-c4cccc(OC(F)(F)F)c4)ccc23)CC1]

tert-Butyl 4-(8-(3-(trifluoromethoxy)phenyl)-2,3,4,5-tetrahydro-1H-1,5-methanobenzo[c]azepine-2-carbonyl)piperidine-1-carboxylate. ¹H NMR (500 MHz, as a mixture of rotamers) δ 7.53-7.38 (comp, 5H), 7.33 (d, J=8.0 Hz, 1H), 7.19 (d, J=8.0 Hz, 1H), 6.00 (br s, 0.5H), 4.20-4.05 (comp, 2H), 3.61 (brs, 0.5H), 3.42-3.35 (m, 1H), 2.95-2.25 (comp, 5H), 2.05-1.99 (m, 1H), 1.89-1.64 (comp, 7H), 1.46 (s, 9H). ¹³C NMR (126 MHz, as a mixture of rotamers) δ 172.4, 154.7, 149.7 (q, J_{C-F}=1.3 Hz), 146.5, 143.1, 142.0, 139.2, 130.1, 127.6, 125.4, 123.2, 122.6, 120.5 (q, J_{C-F}=257.0 Hz), 119.6, 119.5, 79.6, 54.8, 44.0, 43.3, 39.8, 38.9, 31.1, 29.7, 28.7, 28.4. HRMS (ESI) m/z calcd for C₂₉H₃₃F₃N₂NaO₄ (M+Na)⁺, 553.2285. found 553.2288.

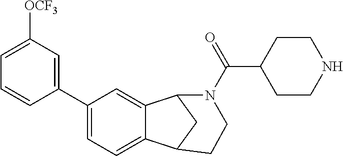 [SMILES: O=C(C1CCNCC1)N1CCC2CC1c1cc(-c3cccc(OC(F)(F)F)c3)ccc12]

Piperidin-4-yl(8-(3-(trifluoromethoxy)phenyl)-1,3,4,5-tetrahydro-2H-1,5-methanobenzo[c]azepin-2-yl)methanone. ¹H NMR (400 MHz, as a mixture of rotamers) δ 7.53-7.13 (comp, 7H), 5.99 (d, J=4.0 Hz, 0.65H), 5.20 (d, J=4.0 Hz, 0.35H), 4.30 (dd, J=14.0, 6.4 Hz, 0.35H), 3.61 (dd, J=14.0, 6.4 Hz, 0.65H), 3.41-3.33 (m, 1H), 3.30-3.09 (comp, 2H), 2.99-2.45 (comp, 5H), 2.34-2.22 (m, 1H), 2.05-1.94 (m, 1H), 1.93-1.56 (comp, 6H). ¹³C NMR (126 MHz, as a mixture of rotamers) δ 172.7, 172.4, 149.6, 146.5, 146.4, 143.10, 143.05, 142.1, 139.2, 139.0, 130.1, 130.0, 128.0, 127.4, 125.5, 125.3, 123.3, 123.0, 122.7, 122.2, 120.5 (q, J_{C-F}=253.3 Hz), 119.7, 119.6, 119.4, 58.7, 54.6, 45.5, 45.4, 45.3, 44.4, 43.9, 39.9, 39.8, 39.7, 38.6, 36.4, 31.2, 30.5. HRMS (ESI) m/z calcd for C₂₄H₂₆F₃N₂O₂ (M+H)⁺, 431.1941. found 431.1949.

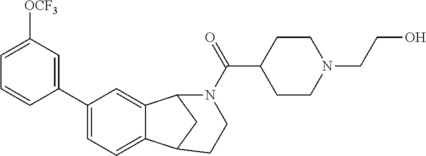 [SMILES: O=C(C1CCN(CCO)CC1)N1CCC2CC1c1cc(-c3cccc(OC(F)(F)F)c3)ccc12]

(1-(2-Hydroxyethyl)piperidin-4-yl)(8-(3-(trifluoromethoxy)phenyl)-1,3,4,5-tetrahydro-2H-1,5-methanobenzo[c]azepin-2-yl)methanone. ¹H NMR (400 MHz, as a mixture of rotamers) δ 7.53-7.30 (comp, 6H), 7.21-7.15 (m, 1H), 5.94 (d, J=4.0 Hz, 0.65H), 5.13 (d, J=4.0 Hz, 0.35H), 4.25 (dd, J=14.0, 6.6 Hz, 0.35H), 4.05-3.72 (comp, 2.65H), 3.65-3.23 (comp, 7H), 3.19-3.14 (m, 1H), 3.00-2.41 (comp, 3H), 2.38-1.94 (comp, 5H), 2.07-1.94 (m, 2H), 1.90-1.79 (m, 1H), 1.70 (d, J=10.4 Hz, 1H). ¹³C NMR (126 MHz, as a mixture of rotamers) δ 149.7, 146.3, 146.1, 143.01, 142.98, 141.6, 139.33, 139.28, 130.2, 130.1, 128.2, 127.8, 125.5, 125.4, 123.5, 123.3, 122.6, 122.0, 120.5 (q, J_{C-F}=257.0 Hz), 119.7, 119.6, 60.8, 56.1, 54.8, 45.9, 44.4, 43.8, 40.3, 39.7, 39.6, 31.0, 29.7. HRMS (ESI) m/z calcd for C₂₆H₃₀F₃N₂O₃ (M+H)⁺, 475.2203. found 475.2219.

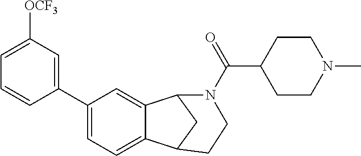 [SMILES: CN1CCC(C(=O)N2CCC3CC2c2cc(-c4cccc(OC(F)(F)F)c4)ccc23)CC1]

(1-Methylpiperidin-4-yl)(8-(3-(trifluoromethoxy)phenyl)-1,3,4,5-tetrahydro-2H-1,5-methanobenzo[c]azepin-2-yl)methanone. ¹H NMR (500 MHz, as a mixture of rotamers) δ 7.54-7.31 (comp, 6H), 7.22-7.17 (m, 1H), 5.99 (d, J=4.0 Hz, 0.65H), 5.14 (d, J=4.0 Hz, 0.35H), 4.32-3.54 (comp, 2H), 3.39 (s, 1H), 3.32-3.00 (comp, 2H), 2.78-2.69 (m, 1H), 2.57-2.20 (comp, 6H), 2.07-1.91 (comp, 4H), 1.83 (d, J=10.8 Hz, 1H), 1.74-1.69 (m, 1H), 1.65-1.57 (m, 1H). ¹³C NMR (126 MHz, as a mixture of rotamers) δ 172.6, 172.3, 149.7, 146.5, 143.1, 141.9, 139.2, 130.2, 130.1, 128.1, 127.6, 125.5, 125.4, 123.4, 123.1, 122.7, 122.2, 120.5 (q, J_{C-F}=258.3 Hz), 119.7, 119.61, 119.56, 59.0, 54.7, 44.5, 39.8, 39.7, 36.4, 31.9, 29.7. HRMS (ESI) m/z calcd for C₂₅H₂₈F₃N₂O₂ (M+H)⁺, 445.2097. found 445.2103.

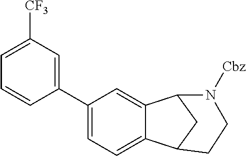 [SMILES: O=C(OCc1ccccc1)N1CCC2CC1c1cc(-c3cccc(C(F)(F)F)c3)ccc12]

Benzyl 8-(3-(trifluoromethyl)phenyl)-1,3,4,5-tetrahydro-2H-1,5-methanobenzo[c]azepine-2-carboxylate. ¹H NMR (500 MHz, as a mixture of rotamers) δ 7.84-7.28 (comp, 11H), 5.60 (br s, 0.5H), 5.48 (br s, 0.5H), 5.28-5.06 (comp, 2H), 3.94-3.78 (m, 1H), 3.37-3.22 (m, 1H), 2.57-2.40 (m, 1H), 2.36-2.20 (m, 1H), 2.13-1.98 (m, 1H), 1.92 (d, J=11.0 Hz, 1H), 1.74-1.59 (m, 1H); ¹³C NMR (126 MHz, as a mixture of rotamers) δ 155.0, 154.8, 146.3, 142.1, 141.9, 139.1, 136.9, 136.8, 131.2 (q, J_{C-F}=32.8 Hz), 130.4, 129.2, 128.5, 127.9, 127.6, 127.4, 125.3, 123.8 (m), 123.2, 122.7, 122.0 (q, J_{C-F}=272.2 Hz), 122.5, 67.1, 57.6, 57.3, 43.7, 39.5, 38.6, 30.2. HRMS (ESI) m/z calcd for C₂₆H₂₂F₃NNaO₂ (M+Na)⁺, 460.1495. found 460.1501.

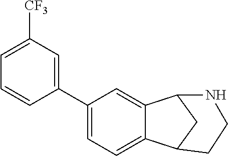 [SMILES: FC(F)(F)c1cccc(-c2ccc3c(c2)C2CC3CCN2)c1]

8-(3-(Trifluoromethyl)phenyl)-2,3,4,5-tetrahydro-1H-1,5-methanobenzo[c]azepine. ¹H NMR (500 MHz) δ 7.84 (s, 1H), 7.76 (d, J=8.0 Hz, 1H), 7.61-7.43 (comp, 4H), 7.28 (d, J=8.0 Hz, 1H), 4.31 (d, J=4.0 Hz, 1H), 3.26-3.21 (m, 1H), 2.92 (dd, J=6.0, 4.0 Hz, 1H), 2.36 (td, J=12.0, 4.0 Hz, 1H), 2.28-2.22 (m, 1H), 2.03-1.96 (comp, 2H), 1.61-1.54 (m, 1H); ¹³C NMR (126 MHz) δ 146.3, 143.5, 142.2, 138.8, 131.0 (q, J_{C-F}=31.5 Hz), 130.2, 129.1, 126.8, 123.8 (q, J_{C-F}=3.8 Hz), 123.6 (q, J_{C-F}=3.8 Hz), 123.4 (q, J_{C-F}=272.2 Hz), 122.8, 121.8, 58.6, 45.1, 39.9, 38.9, 30.9. HRMS (ESI) m/z calcd for C₁₈H₁₇F₃N (M+H)⁺, 304.1308. found 304.1312.

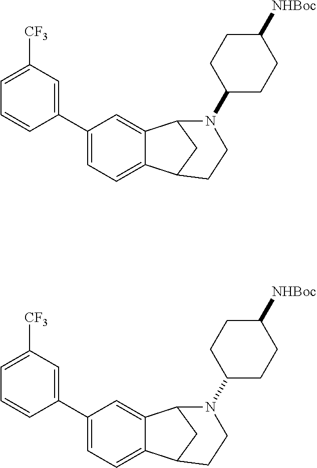

tert-Butyl (4-(8-(3-(trifluoromethyl)phenyl)-1,3,4,5-tetrahydro-2H-1,5-methanobenzo[c]azepin-2-yl)cyclohexyl)carbamate (cis-isomer and trans-isomer). For cis-isomer. ¹H NMR (500 MHz) δ 7.81 (s, 1H), 7.75 (d, J=7.5 Hz, 1H), 7.61-7.52 (comp, 2H), 7.44 (d, J=7.5, 1H), 7.31-7.27 (comp, 2H), 4.769 (br s, 1H), 4.39 (d, J=3.0 Hz, 1H), 3.68 (br s, 1H), 3.16 (s, 1H), 2.83 (q, J=5.0 Hz, 1H), 2.30-2.21 (m, 1H), 2.14-2.07 (m, 1H), 2.03-1.89 (comp, 3H), 1.87-1.75 (comp, 2H), 1.72-1.44 (comp, 7H), 1.44 (s, 9H); ¹³C NMR (126 MHz) δ 155.2, 146.8, 142.4, 140.5, 138.1, 131.1 (q, J_{C-F}=32.2 Hz), 130.5, 129.2, 126.7, 124.2 (q, J_{C-F}=272.2 Hz), 123.9 (q, J_{C-F}=3.8 Hz), 123.6 (q, J_{C-F}=3.8 Hz), 122.9, 122.7, 78.9, 58.7, 57.4, 46.7, 44.5, 43.5, 39.6, 30.6, 29.7, 28.5, 28.4, 26.5, 25.9; HRMS (ESI) m/z calcd for C₂₉H₃₆F₃N₂O₂ (M+H)⁺, 501.2723. found 501.2721. For trans-isomer. ¹H NMR (500 MHz) δ 7.81 (s, 1H), 7.75 (d, J=7.5 Hz, 1H), 7.62-7.53 (comp, 2H), 7.46 (d, J=7.5, 1H), 7.32-7.27 (comp, 2H), 4.41 (d, J=4.8 Hz, 1H), 4.34 (br s, 1H), 3.42 (br s, 1H), 3.16 (s, 1H), 2.85 (q, J=5.0 Hz, 1H), 2.42-2.33 (m, 1H), 2.28-2.21 (m, 1H), 2.16-2.09 (m, 1H), 2.04-1.85 (comp, 5H), 1.64-1.55 (comp, 2H), 1.43 (s, 9H), 1.40-1.25 (comp, 2H), 1.20-0.99 (comp, 2H); ¹³C NMR (126 MHz) δ 155.2, 147.0, 142.5, 140.8, 138.1, 131.3 (q, J_{C-F}=32.2 Hz), 130.4, 129.2, 126.7, 124.2 (q, J_{C-F}=272.2 Hz), 123.9 (q, J_{C-F}=3.8 Hz), 123.7 (q, J_{C-F}=3.8 Hz), 122.9, 79.1, 60.3, 59.5, 49.3, 44.7, 44.0, 39.7, 32.3, 30.5, 29.5, 29.1, 28.4; HRMS (ESI) m/z calcd for C₂₉H₃₆F₃N₂O₂ (M+H)⁺, 501.2723. found 501.2723.

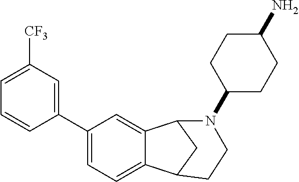

4-(8-(3-(trifluoromethyl)phenyl)-1,3,4,5-tetrahydro-2H-1,5-methanobenzo[c]azepin-2-yl)cyclohexan-1-amine. ¹H NMR (500 MHz) δ 7.81 (s, 1H), 7.75 (d, J=7.5 Hz, 1H), 7.61-7.51 (comp, 2H), 7.44 (dd, J=7.5, 1.5 Hz, 1H), 7.32-7.27 (comp, 2H), 4.39 (d, J=5.0 Hz, 1H), 3.15 (s, 1H), 2.91 (br s, 1H), 2.84 (q, J=5.0 Hz, 1H), 2.29-2.22 (m, 1H), 2.14-2.08 (m, 1H), 2.06-1.97 (comp, 2H), 1.95 (d, J=10.5 Hz, 1H), 1.81-1.40 (comp, 11H). ¹³C NMR (126 MHz) δ 146.9, 142.5, 140.8, 138.0, 131.1 (q, J_{C-F}=32.2 Hz), 130.5, 129.1, 126.6, 124.2 (q, J_{C-F}=272.2 Hz), 123.9 (q, J_{C-F}=3.8 Hz), 123.6 (q, J_{C-F}=3.8 Hz), 122.8, 122.7, 58.7, 57.5, 48.0, 44.5, 43.6, 39.7, 30.6, 26.4, 25.9. HRMS (ESI) m/z calcd for C₂₄H₂₈F₃N₂ (M+H)⁺, 401.2199. found 401.2193.

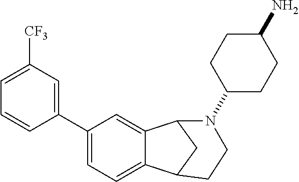

4-(8-(3-(trifluoromethyl)phenyl)-1,3,4,5-tetrahydro-2H-1,5-methanobenzo[c]azepin-2-yl)cyclohexan-1-amine. ¹H NMR (500 MHz) δ 7.81 (s, 1H), 7.75 (d, J=7.5 Hz, 1H), 7.63-7.54 (comp, 2H), 7.45 (dd, J=7.5, 1.5 Hz, 1H), 7.35-7.28 (comp, 2H), 4.46 (d, J=3.8 Hz, 1H), 3.18 (s, 1H), 2.98-2.86 (m, 1H), 2.77-2.62 (m, 1H), 2.43-2.35 (m, 1H), 2.30-2.22 (m, 1H), 2.15-1.80 (comp, 7H), 1.67-1.57 (comp, 2H), 1.45-1.36 (m, 1H), 1.24-0.99 (comp, 4H). ¹³C NMR (126 MHz) δ 147.2, 142.6, 140.8, 138.5, 131.3 (q, J_{C-F}=32.2 Hz), 130.7, 129.5, 127.2, 124.5 (q, J_{C-F}=272.2 Hz), 124.2 (q, J_{C-F}=3.8 Hz), 124.0 (q, J_{C-F}=3.8 Hz), 123.23, 123.18, 60.8, 59.9, 50.3, 44.4, 39.8, 35.7, 30.6, 29.7, 29.2. HRMS (ESI) m/z calcd for C₂₄H₂₈F₃N₂ (M+H)⁺, 401.2199. found 401.2201.

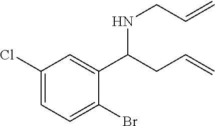 [SMILES: C=CCNC(CC=C)c1cc(Cl)ccc1Br]

N-Allyl-1-(2-bromo-5-chlorophenyl)but-3-en-1-amine. ¹H NMR (500 MHz) δ 7.58 (d, J=3.5 Hz, 1H), 7.42 (d, J=11.0 Hz, 1H), 7.10-7.04 (m, 1H), 5.92-5.68 (comp, 2H), 5.17-5.02 (comp, 4H), 4.15 (dd, J=10.0, 5.0 Hz, 1H), 3.14-2.94 (comp, 2H), 2.53-2.41 (m, 1H), 2.25-2.12 (m, 1H), 1.55 (br s, 1H). ¹³C NMR (126 MHz) δ 144.4, 136.4, 134.5, 133.8, 133.7, 128.4, 128.3, 121.5, 118.3, 115.9, 59.5, 50.0, 41.2. HRMS (ESI) m/z calcd for C₁₃H₁₆BrClN (M+H)⁺ 300.1049. found 300.1050.

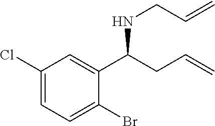 [SMILES: C=CCN[C@@H](CC=C)c1cc(Cl)ccc1Br]

(S)—N-Allyl-1-(2-bromo-5-chlorophenyl)but-3-en-1-amine. (R)-mandelic acid (6.7 g, 44 mmol) was added to a solution of N-allyl-1-(2-bromo-5-chlorophenyl)but-3-en-1-amine (13.2 g, 44 mmol) in EtOAc (40 mL), and the resulting mixture heated to 60° C. for 30 min. The solvent was then removed under reduced pressure, and the precipitated solid was recrystallized with isopropanol/ethanol (7:3 v/v). After filtration, enantioenriched crystals were collected and subjected to 3-5 rounds of recrystallization with isopropanol/ethanol (7:3 v/v) to give the pure diastereomeric salt (dr>20:1). This solid was dissolved in CH₂Cl₂ (20 mL), and 1N NaOH was added to neutralize the salt. The mixture was extracted with CH₂Cl₂ (2×20 mL). The combined organic extracts were dried (MgSO₄), concentrated to afford 1.82 g (27%) of (S)-isomer as a colourless oil. ¹H NMR (500 MHz) δ 7.58 (d, J=3.5 Hz, 1H), 7.42 (d, J=11.0 Hz, 1H), 7.10-7.04 (m, 1H), 5.92-5.68 (comp, 2H), 5.17-5.02 (comp, 4H), 4.15 (dd, J=10.0, 5.0 Hz, 1H), 3.14-2.94 (comp, 2H), 2.53-2.41 (m, 1H), 2.25-2.12 (m, 1H), 1.55 (br s, 1H). ¹³C NMR (126 MHz) δ 144.4, 136.4, 134.5, 133.8, 133.7, 128.4, 128.3, 121.5, 118.3, 115.9, 59.5, 50.0, 41.2. HRMS (ESI) m/z calcd for C₁₃H₁₆BrClN (M+H)⁺ 300.1049. found 300.1050.

$${[ α ]}_{D}^{25} = - 65.7 °$$

(c=0.67, CHCl₃).

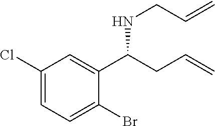 [SMILES: C=CCN[C@H](CC=C)c1cc(Cl)ccc1Br]

(R)—N-Allyl-1-(2-bromo-5-chlorophenyl)but-3-en-1-amine. The mother liquors from above crystallization for(S)-isomer were collected and neutralized with 1N NaOH. The residue (11.4 g, 38 mmol) was re-acidified with(S)-mandelic acid (5.8 g, 38 mmol) to obtain solid. Recrystallization of the solid with isopropanol/ethanol (7:3 v/v, 3 times) provided the pure diastereomeric salt (dr>20:1). This solid was dissolved in CH₂Cl₂ (20 mL), and IN NaOH was added to neutralize the salt. The mixture was extracted with CH₂Cl₂ (2×20 mL). The combined organic extracts were dried (MgSO₄), concentrated to afford 2.35 g (36%) of (R)-isomer as a colourless oil. ¹H NMR (500 MHz) δ 7.58 (d, J=3.5 Hz, 1H), 7.42 (d, J=11.0 Hz, 1H), 7.10-7.04 (m, 1H), 5.92-5.68 (comp, 2H), 5.17-5.02 (comp, 4H), 4.15 (dd, J=10.0, 5.0 Hz, 1H), 3.14-2.94 (comp, 2H), 2.53-2.41 (m, 1H), 2.25-2.12 (m, 1H), 1.55 (br s, 1H). ¹³C NMR (126 MHz) δ 144.4, 136.4, 134.5, 133.8, 133.7, 128.4, 128.3, 121.5, 118.3, 115.9, 59.5, 50.0, 41.2. HRMS (ESI) m/z calcd for C₁₃H₁₆BrClN (M+H)⁺ 300.1049. found 300.1050.

$${[ α ]}_{D}^{25} = + 61.8 °$$

(c=0.68, CHCl₃).

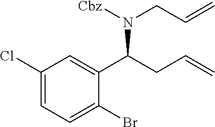 [SMILES: C=CC[C@@H](c1cc(Cl)ccc1Br)N(CC=C)C(=O)OCc1ccccc1]

Benzyl (S)-allyl(1-(2-bromo-5-chlorophenyl)but-3-en-1-yl)carbamate. ¹H NMR (400 MHz) δ 7.46 (d, J=8.4 Hz, 1H), 7.43-7.28 (comp, 6H), 7.13 (dd, J=8.4, 2.4 Hz, 1H), 5.73 (br s, 1H), 5.60 (m, 1H), 5.44 (t, J=8.0 Hz, 1H), 5.26-4.98 (comp, 4H), 4.94-4.79 (comp, 2H), 3.79-3.69 (m, 1H), 3.68-3.55 (m, 1H), 2.88-2.58 (comp, 2H); ¹³C NMR (126 MHz) δ 155.9, 140.3, 136.6, 134.5, 134.3, 134.0, 133.3, 129.2, 128.4, 127.9, 123.7, 118.1, 116.3, 67.3, 58.5, 46.9, 36.3. HRMS (ESI) m/z calcd for C₂₁H₂₂NO₂ClBr (M+H)⁺ 434.0522. found 434.0524.

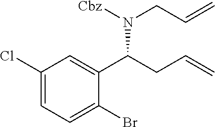 [SMILES: C=CC[C@H](c1cc(Cl)ccc1Br)N(CC=C)C(=O)OCc1ccccc1]

Benzyl (R)-allyl(1-(2-bromo-5-chlorophenyl)but-3-en-1-yl)carbamate. ¹H NMR (400 MHz) δ 7.46 (d, J=8.4 Hz, 1H), 7.43-7.28 (comp, 6H), 7.13 (dd, J=8.4, 2.4 Hz, 1H), 5.73 (br s, 1H), 5.60 (m, 1H), 5.44 (t, J=8.0 Hz, 1H), 5.26-4.98 (comp, 4H), 4.94-4.79 (comp, 2H), 3.79-3.69 (m, 1H), 3.68-3.55 (m, 1H), 2.88-2.58 (comp, 2H); ¹³C NMR (126 MHz) δ 155.9, 140.3, 136.6, 134.5, 134.3, 134.0, 133.3, 129.2, 128.4, 127.9, 123.7, 118.1, 116.3, 67.3, 58.5, 46.9, 36.3. HRMS (ESI) m/z calcd for C₂₁H₂₂NO₂ClBr (M+H)⁺ 434.0522. found 434.0524.

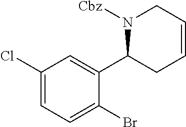 [SMILES: O=C(OCc1ccccc1)N1CC=CC[C@H]1c1cc(Cl)ccc1Br]

Benzyl (S)-2-(2-bromo-5-chlorophenyl)-3,6-dihydropyridine-1(2H)-carboxylate. ¹H NMR (400 MHz) δ 7.45 (d, J=8.4 Hz, 1H), 7.38-7.19 (comp, 5H), 7.13-7.05 (comp, 2H), 6.04-5.58 (comp, 3H), 5.23-5.04 (comp, 2H), 4.34-4.23 (m, 1H), 3.99-3.87 (m, 1H), 2.79-2.66 (m, 1H), 2.51-2.30 (m, 1H); ¹³C NMR (100 MHz) δ 155.4, 144.2, 136.5, 134.1, 133.4, 128.6, 127.9, 127.7, 124.3, 122.7, 120.6, 67.3, 52.1, 42.4, 28.3. HRMS (ESI) m/z calcd for C₁₉H₁₇BrClNNaO₂ (M+Na)⁺ 428.0023. found 428.0021.

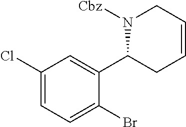 [SMILES: O=C(OCc1ccccc1)N1CC=CC[C@@H]1c1cc(Cl)ccc1Br]

Benzyl (R)-2-(2-bromo-5-chlorophenyl)-3,6-dihydropyridine-1(2H)-carboxylate. ¹H NMR (400 MHz) δ 7.45 (d, J=8.4 Hz, 1H), 7.38-7.19 (comp, 5H), 7.13-7.05 (comp, 2H), 6.04-5.58 (comp, 3H), 5.23-5.04 (comp, 2H), 4.34-4.23 (m, 1H), 3.99-3.87 (m, 1H), 2.79-2.66 (m, 1H), 2.51-2.30 (m, 1H); ¹³C NMR (100 MHz) δ 155.4, 144.2, 136.5, 134.1, 133.4, 128.6, 127.9, 127.7, 124.3, 122.7, 120.6, 67.3, 52.1, 42.4, 28.3. HRMS (ESI) m/z calcd for C₁₉H₁₇BrClNNaO₂ (M+Na)⁺ 428.0023. found 428.0021.

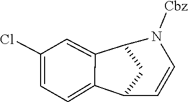

Benzyl (1S,5S)-8-chloro-1,5-dihydro-2H-1,5-methanobenzo[c]azepine-2-carboxylate. ¹H NMR (400 MHz, as a mixture of rotamers) δ 7.50-7.28 (comp, 5.5H), 7.20-7.13 (comp, 1.5H), 7.09 (d, J=8.0 Hz, 1H), 6.50 (d, J=8.0 Hz, 0.4H), 6.40 (d, J=8.0 Hz, 0.6H), 5.58 (d, J=3.6 Hz, 0.6H), 5.45 (d, J=3.6 Hz, 0.4H), 5.37-5.30 (m, 1H), 5.27-5.08 (comp, 2H), 3.33 (m, 1H), 2.42-2.33 (m, 0.6H), 2.33-2.26 (m, 0.4H), 2.13-2.06 (m, 1H). ¹³C NMR (100 MHz, as a mixture of rotamers) δ 152.0, 151.9, 147.1, 147.0, 140.6, 140.4, 136.0, 135.8, 132.2, 132.0, 128.7, 128.5, 128.3, 128.2, 128.1, 124.3, 123.9, 122.0, 121.8, 121.7, 121.6, 67.8, 67.7, 57.7, 57.3, 37.9, 37.8, 37.7, 37.4. HRMS m/z calcd for C₁₉H₁₆ClNNaO₂ (M+Na)⁺ 348.0762. found 348.0761.

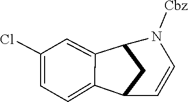

Benzyl (1R,5R)-8-chloro-1,5-dihydro-2H-1,5-methanobenzo[c]azepine-2-carboxylate. ¹H NMR (400 MHz, as a mixture of rotamers) δ 7.50-7.28 (comp, 5.5H), 7.20-7.13 (comp, 1.5H), 7.09 (d, J=8.0 Hz, 1H), 6.50 (d, J=8.0 Hz, 0.4H), 6.40 (d, J=8.0 Hz, 0.6H), 5.58 (d, J=3.6 Hz, 0.6H), 5.45 (d, J=3.6 Hz, 0.4H), 5.37-5.30 (m, 1H), 5.27-5.08 (comp, 2H), 3.33 (m, 1H), 2.42-2.33 (m, 0.6H), 2.33-2.26 (m, 0.4H), 2.13-2.06 (m, 1H). ¹³C NMR (100 MHz, as a mixture of rotamers δ 152.0, 151.9, 147.1, 147.0, 140.6, 140.4, 136.0, 135.8, 132.2, 132.0, 128.7, 128.5, 128.3, 128.2, 128.1, 124.3, 123.9, 122.0, 121.8, 121.7, 121.6, 67.8, 67.7, 57.7, 57.3, 37.9, 37.8, 37.7, 37.4. HRMS m/z calcd for C₁₉H₁₆ClNNaO₂ (M+Na)⁺ 348.0762. found 348.0761.

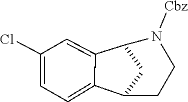

Benzyl (1S,5R)-8-chloro-1,3,4,5-tetrahydro-2H-1,5-methanobenzo[c]azepine-2-carboxylate (1-078). Prepared from 1-071 (1.40 g, 4.3 mmol) according to same procedure for 1-061 affording 0.99 g (70%) of 1-078 as a colorless oil after purification via flash column chromatography (SiO₂), eluting with a mixture of hexane/EtOAc (100:0 to 10:1 v/v). ¹H NMR (500 MHz, as a mixture of rotamers) δ 7.48-7.27 (comp, 5.5H), 7.24-7.21 (m, 1H), 7.207.12 (comp, 1.5H), 5.50 (br s, 0.5H), 5.38 (br s, 0.5H), 5.25-5.07 (comp, 2H), 3.76-3.91 (m, 1H), 3.29-3.23 (m, 1H), 2.51-2.35 (m, 1H), 2.27-2.13 m, 1H), 2.05-1.93 (m, 1H), 1.87 (d, J=11.0 Hz, 1H), 1.63-1.52 (m, 1H); ¹³C NMR (126 MHz, as a mixture of rotamers) δ 154.9, 154.7, 144.7, 142.9, 142.7, 136.8, 136.7, 132.6, 128.4, 128.0, 127.94, 127.86, 124.2, 124.0, 123.9, 123.8, 67.1, 57.4, 57.2, 43.6, 39.3, 38.5, 30.1. HRMS (ESI) m/z calcd for C₁₉H₁₉NO₂Cl (M+H)⁺ 328.1104. found 328.1106.

$${[ α ]}_{D}^{25} = - 162.5 °$$

(c=0.72, CHCl₃).

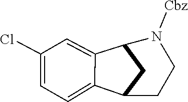

Benzyl (1R,5S)-8-chloro-1,3,4,5-tetrahydro-2H-1,5-methanobenzo[c]azepine-2-carboxylate. ¹H NMR (500 MHz, as a mixture of rotamers) δ 7.48-7.27 (comp, 5.5H), 7.24-7.21 (m, 1H), 7.207.12 (comp, 1.5H), 5.50 (br s, 0.5H), 5.38 (br s, 0.5H), 5.25-5.07 (comp, 2H), 3.76-3.91 (m, 1H), 3.29-3.23 (m, 1H), 2.51-2.35 (m, 1H), 2.27-2.13 m, 1H), 2.05-1.93 (m, 1H), 1.87 (d, J=11.0 Hz, 1H), 1.63-1.52 (m, 1H); ¹³C NMR (126 MHz, as a mixture of rotamers) δ 154.9, 154.7, 144.7, 142.9, 142.7, 136.8, 136.7, 132.6, 128.4, 128.0, 127.94, 127.86, 124.2, 124.0, 123.9, 123.8, 67.1, 57.4, 57.2, 43.6, 39.3, 38.5, 30.1. HRMS (ESI) m/z calcd for C₁₉H₁₉NO₂Cl (M+H)⁺ 328.1104. found 328.1106.

$${[ α ]}_{D}^{25} = + 150. °$$

(c=0.60, CHCl₃).

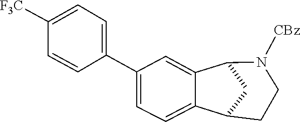

Benzyl (1S,5R)-8-(4-(trifluoromethyl)phenyl)-1,3,4,5-tetrahydro-2H-1,5-methanobenzo[c]azepine-2-carboxylate. ¹H NMR (500 MHz, as a mixture of rotamers) δ 7.72-7.24 (comp, 12H), 5.61 (br s, 0.5H), 5.49 (br s, 0.5H), 5.27-5.07 (comp, 2H), 3.96-3.81 (m, 1H), 3.39-3.24 (m, 1H), 2.61-2.43 (m, 1H), 2.34-2.20 (m, 1H), 2.12-2.00 (m, 1H), 1.94 (d, J=11.0 Hz, 1H), 1.74-1.59 (m, 1H). ¹³C NMR (126 MHz, as a mixture of rotamers) δ 155.0, 154.8, 146.5, 144.6, 142.1, 141.9, 139.0, 136.9, 136.8, 129.2 (q, J_{C-F}=31.5 Hz), 128.4, 127.9, 127.8, 127.3, 125.6 (q, J_{C-F}=3.8 Hz), 123.3 (q, J_{C-F}=272.2 Hz), 123.2, 122.7, 122.5, 67.0, 57.6, 57.3, 43.6, 39.5, 38.6, 30.2. HRMS (ESI) m/z calcd for C₂₆H₂₂F₃NNaO₂ (M+Na)⁺, 460.1495. found 460.1499.

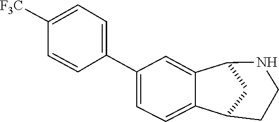

(1S,5R)-8-(4-(Trifluoromethyl)phenyl)-2,3,4,5-tetrahydro-1H-1,5-methanobenzo[c]azepine. ¹H NMR (500 MHz) δ 7.76 (s, 1H), 7.68 (q, J=8.5 Hz, 4H), 7.56 (dd, J=7.5, 1.5 Hz, 1H), 7.34 (d, J=8.0 Hz, 1H), 7.11 (br s, 1H), 4.67 (d, J=2.5 Hz, 1H), 3.38 (s, 1H), 3.07 (dd, J=13.0, 5.5 Hz, 1H), 2.49 (td, J=12.5, 5.0 Hz, 1H), 2.42-2.33 (comp, 2H), 2.22 (td, J=12.5, 5.0 Hz, 1H), 1.67 (d, J=13.5 Hz, 1H). ¹³C NMR (126 MHz) δ 146.1, 144.2, 139.6, 139.1, 129.3 (q, J_{C-F}=32.5 Hz), 128.6, 127.4, 125.7 (q, J_{C-F}=3.8 Hz), 124.2 (q, J_{C-F}=272.2 Hz), 123.2, 123.1, 58.0, 42.7, 38.8, 38.1, 28.3. HRMS (ESI) m/z calcd for C₁₈H₁₇F₃N (M+H)⁺, 304.1308. found 304.1309.

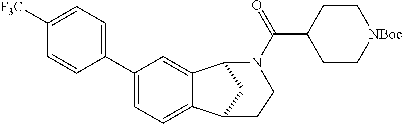

tert-Butyl 4-((1S,5R)-8-(4-(trifluoromethyl)phenyl)-2,3,4,5-tetrahydro-1H-1,5-methanobenzo[c]azepine-2-carbonyl)piperidine-1-carboxylate. ¹H NMR (500 MHz, as a mixture of rotamers) δ 7.63-7.28 (comp, 6H), 7.19 (d, J=8.4 Hz, 1H), 6.01 (d, J=4.0 Hz, 0.65H), 5.19 (d, J=4.0 Hz, 0.35H), 4.32 (dd, J=13.6, 6.4 Hz, 0.35H), 3.60 (dd, J=13.6, 6.4 Hz, 0.65H), 3.39 (s, 1H), 2.95-2.61 (comp, 3H), 2.55-2.22 (comp, 2H), 2.04-1.97 (m, 1H), 1.86-1.78 (comp, 2H), 1.74-1.66 (comp, 3H), 1.64-1.50 (comp, 3H), 1.46 (d, J=8.8 Hz, 9H). ¹³C NMR (126 MHz, as a mixture of rotamers) δ 172.4, 171.1, 154.7, 146.7, 144.4, 142.0, 139.2, 129.3 (q, J_{C-F}=32.8 Hz), 127.7, 127.3, 125.7 (q, J_{C-F}=3.8 Hz), 124.2 (q, J_{C-F}=272.8 Hz), 123.2, 122.7, 79.5, 54.6, 44.0, 43.3, 39.8, 38.9, 31.1, 29.7, 28.7, 28.4. HRMS (ESI) m/z calcd for C₂₉H₃₃F₃N₂NaO₃ (M+Na)⁺, 537.2335. found 537.2339.

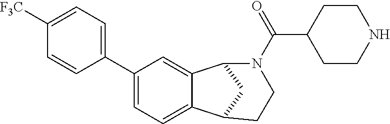

Piperidin-4-yl((1S,5R)-8-(4-(trifluoromethyl)phenyl)-1,3,4,5-tetrahydro-2H-1,5-methanobenzo[c]azepin-2-yl)methanone. ¹H NMR (400 MHz, as a mixture of rotamers) δ 7.72-7.62 (comp, 4H), 7.56-7.40 (comp, 2H), 7.38-7.31 (m, 1H), 6.00 (d, J=4.0 Hz, 0.65H), 5.19 (d, J=4.0 Hz, 0.35H), 4.30 (dd, J=14.0, 6.4 Hz, 0.35H), 3.37 (s, 1H), 3.27-3.07 (comp, 2H), 2.95-2.51 (comp, 3.65H), 2.25-2.30 (comp, 3H) 2.05-1.96 (m, 1H), 1.86-1.79 (comp, 2H), 1.77-1.66 (comp, 3H). ¹³C NMR (126 MHz, as a mixture of rotamers) δ 171.1, 171.0, 146.6, 146.4, 144.3, 141.7, 140.8, 139.4, 139.3, 129.3 (q, J_{C-F}=32.3 Hz), 128.3, 127.8, 127.4, 127.3, 125.7 (q, J_{C-F}=3.8 Hz), 124.2 (q, J_{C-F}=272.2 Hz), 123.4, 123.2, 122.8, 122.4, 59.1, 54.9, 43.8, 42.8, 42.6, 40.2, 39.6, 35.1, 31.1. HRMS (ESI) m/z calcd for C₂₄H₂₆F₃N₂O (M+H)⁺, 415.1992. found 415.2004.

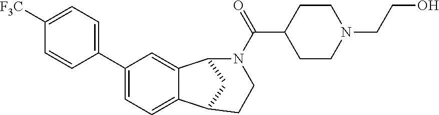

(1-(2-Hydroxyethyl)piperidin-4-yl)((1S,5R)-8-(4-(trifluoromethyl)phenyl)-1,3,4,5-tetrahydro-2H-1,5-methanobenzo[c]azepin-2-yl)methanone. ¹H NMR (500 MHz, as a mixture of rotamers) δ 7.72-7.62 (comp, 4H), 7.56-7.31 (comp, 3H), 5.99 (d, J=4.0 Hz, 0.65H), 5.17 (d, J=4.0 Hz, 0.35H), 4.30 (dd, J=14.0, 6.5 Hz, 0.35H), 3.78-3.68 (comp, 2H), 3.56 (dd, J=14.0, 6.5 Hz, 0.65H), 3.43-3.36 (m, 1H), 3.25-3.07 (comp, 2H), 2.95-2.20 (comp, 7H), 2.14-1.80 (comp, 6H), 1.74-1.66 (m, 1H). ¹³C NMR (126 MHz, as a mixture of rotamers) δ 172.4, 172.1, 146.7, 146.5, 144.4, 142.0, 141.0, 139.3, 139.2, 129.3 (q, J_{C-F}=32.8 Hz), 128.2, 127.6, 127.4, 127.3, 125.7 (q, J_{C-F}=3.8 Hz), 124.2 (q, J_{C-F}=272.2 Hz), 123.4, 123.1, 122.8, 122.3, 59.8, 58.7, 57.3, 57.2, 54.7, 53.4, 52.5, 52.4, 52.3, 44.4, 43.8, 40.0, 39.8, 39.7, 36.4, 31.2, 30.5. HRMS (ESI) m/z calcd for C₂₆H₃₀F₃N₂O₂ (M+H)⁺, 459.2263. found 459.2255.

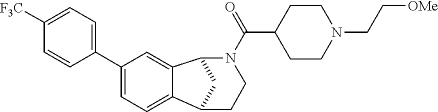

(1-(2-Methoxyethyl)piperidin-4-yl)((1S,5R)-8-(4-(trifluoromethyl)phenyl)-1,3,4,5-tetrahydro-2H-1,5-methanobenzo[c]azepin-2-yl)methanone. ¹H NMR (500 MHz, as a mixture of rotamers) δ 7.72-7.62 (comp, 4H), 7.57-7.31 (comp, 3H), 6.01 (d, J=4.0 Hz, 0.65H), 5.16 (d, J=4.0 Hz, 0.35H), 4.29 (dd, J=14.0, 6.0 Hz, 0.35H), 3.65-3.50 (comp, 2.65H), 3.41-3.32 (comp, 4H), 3.22-3.02 (comp, 2H), 2.85-2.60 (comp, 3H), 2.55-2.12 (comp, 4H), 2.09-1.86 (comp, 5H), 1.83 (d, J=11.0 Hz, 1H), 1.72-1.65 (m, 1H). ¹³C NMR (126 MHz, as a mixture of rotamers) δ 171.8, 171.5, 146.8, 146.6, 144.5, 144.4, 142.2, 141.0, 139.3, 139.1, 129.2 (q, J_{C-F}=31.5 Hz), 128.1, 127.5, 127.4, 127.3, 125.7 (q, J_{C-F}=3.8 Hz), 124.2 (q, J_{C-F}=272.2 Hz), 123.4, 123.1, 122.8, 122.3, 69.7, 58.9, 58.8, 57.9, 57.8, 54.6, 53.1, 44.4, 43.9, 39.92, 39.86, 39.7, 36.3, 31.2, 30.5. HRMS (ESI) m/z calcd for C₂₇H₃₂F₃N₂O₂ (M+H)⁺, 473.2410. found 473.2417.

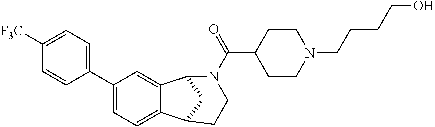

(1-(3-Hydroxypropyl)piperidin-4-yl)((1S,5R)-8-(4-(trifluoromethyl)phenyl)-1,3,4,5-tetrahydro-2H-1,5-methanobenzo[c]azepin-2-yl)methanone. ¹H NMR (500 MHz, as a mixture of rotamers) δ 7.72-7.61 (comp, 4H), 7.55-7.30 (comp, 3H), 5.97 (d, J=4.0 Hz, 0.65H), 5.16 (d, J=4.0 Hz, 0.35H), 4.27 (dd, J=14.0, 6.5 Hz, 0.35H), 3.83-3.75 (comp, 2H), 3.55 (dd, J=14.0, 6.5 Hz, 0.65H), 3.41-3.36 (m, 1H), 3.33-3.21 (comp, 2H), 2.93-2.84 (comp, 2H), 2.80-2.48 (comp, 5H), 2.38-2.20 (comp, 2H), 2.09-1.95 (comp, 3H), 1.92-1.82 (comp, 4H), 1.73-1.66 (m, 1H). ¹³C NMR (126 MHz, as a mixture of rotamers) δ 172.2, 171.8, 146.6, 146.4, 144.4, 141.9, 140.9, 139.3, 139.2, 129.3 (q, J_{C-F}=32.8 Hz), 128.2, 127.7, 127.4, 127.3, 125.7 (q, J_{C-F}=3.8 Hz), 124.2 (q, J_{C-F}=272.2 Hz), 123.4, 123.2, 122.8, 122.3, 61.7, 59.8, 57.1, 54.7, 51.8, 50.7, 44.4, 43.8, 40.1, 39.8, 39.7, 36.5, 31.1, 30.4. HRMS (ESI) m/z calcd for C₂₇H₃₂F₃N₂O₂ (M+H)⁺, 473.2410. found 473.2417.

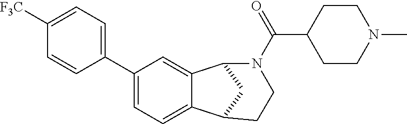

(1-Methylpiperidin-4-yl)((1S,5R)-8-(4-(trifluoromethyl)phenyl)-1,3,4,5-Tetrahydro-2H-1,5-methanobenzo[c]azepin-2-yl)methanone ¹H NMR (500 MHz, as a mixture of rotamers) δ 7.74-7.62 (comp, 4H), 7.57-7.34 (comp, 3H), 5.96 (d, J=4.0 Hz, 0.6H), 5.10 (d, J=4.0 Hz, 0.4H), 4.27 (dd, J=14.5, 6.0 Hz, 0.4H), 3.93-3.47 (m, 2.6H), 3.46-3.18 (m, 5H), 3.76 (s, 3H), 2.42-2.28 (comp, 2H), 2.20-1.95 (comp, 3H), 1.92-1.80 (comp, 2H), 1.76-1.70 (m, 1H). ¹³C NMR (126 MHz, as a mixture of rotamers) δ 172.2, 172.0, 146.5, 146.3, 144.3, 141.7, 140.6, 139.5, 139.4, 129.3 (q, J_{C-F}=32.8 Hz), 128.4, 127.9, 127.4, 127.3, 125.8 (q, J_{C-F}=3.8 Hz), 124.2 (q, J_{C-F}=272.2 Hz), 123.6, 123.3, 122.7, 59.3, 54.7, 44.4, 43.8, 40.4, 39.7, 39.6, 36.5, 31.0, 29.7. HRMS (ESI) m/z calcd for C₂₅H₂₈F₃N₂O (M+H)⁺ 429.2148. found 429.2160.

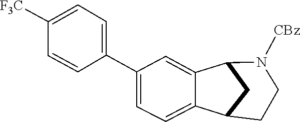

Benzyl (1S,5R)-8-(4-(trifluoromethyl)phenyl)-1,3,4,5-tetrahydro-2H-1,5-methanobenzo[c]azepine-2-carboxylate. ¹H NMR (500 MHz, as a mixture of rotamers) δ 7.72-7.24 (comp, 12H), 5.61 (br s, 0.5H), 5.49 (br s, 0.5H), 5.27-5.07 (comp, 2H), 3.96-3.81 (m, 1H), 3.39-3.24 (m, 1H), 2.61-2.43 (m, 1H), 2.34-2.20 (m, 1H), 2.12-2.00 (m, 1H), 1.94 (d, J=11.0 Hz, 1H), 1.74-1.59 (m, 1H). ¹³C NMR (126 MHz, as a mixture of rotamers) δ 155.0, 154.8, 146.5, 144.6, 142.1, 141.9, 139.0, 136.9, 136.8, 129.2 (q, J_{C-F}=31.5 Hz), 128.4, 127.9, 127.8, 127.3, 125.6 (q, J_{C-F}=3.8 Hz), 123.3 (q, J_{C-F}=272.2 Hz), 123.2, 122.7, 122.5, 67.0, 57.6, 57.3, 43.6, 39.5, 38.6, 30.2. HRMS (ESI) m/z calcd for C₂₆H₂₂F₃NNaO₂ (M+Na)⁺, 460.1495. found 460.1499.

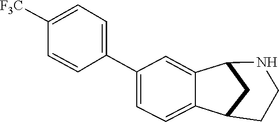

(1S,5R)-8-(4-(Trifluoromethyl)phenyl)-2,3,4,5-tetrahydro-1H-1,5-methanobenzo[c]azepine. ¹H NMR (500 MHz) δ 7.76 (s, 1H), 7.68 (q, J=8.5 Hz, 4H), 7.56 (dd, J=7.5, 1.5 Hz, 1H), 7.34 (d, J=8.0 Hz, 1H), 7.11 (br s, 1H), 4.67 (d, J=2.5 Hz, 1H), 3.38 (s, 1H), 3.07 (dd, J=13.0, 5.5 Hz, 1H), 2.49 (td, J=12.5, 5.0 Hz, 1H), 2.42-2.33 (comp, 2H), 2.22 (td, J=12.5, 5.0 Hz, 1H), 1.67 (d, J=13.5 Hz, 1H). ¹³C NMR (126 MHz) δ 146.1, 144.2, 139.6, 139.1, 129.3 (q, J_{C-F}=32.5 Hz), 128.6, 127.4, 125.7 (q, J_{C-F}=3.8 Hz), 124.2 (q, J_{C-F}=272.2 Hz), 123.2, 123.1, 58.0, 42.7, 38.8, 38.1, 28.3. HRMS (ESI) m/z calcd for C₁₈H₁₇F₃N (M+H)⁺, 304.1308. found 304.1309.

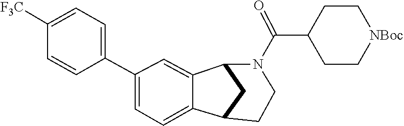

tert-Butyl 4-((1S,5R)-8-(4-(trifluoromethyl)phenyl)-2,3,4,5-tetrahydro-1H-1,5-methanobenzo[c]azepine-2-carbonyl)piperidine-1-carboxylate. ¹H NMR (500 MHz, as a mixture of rotamers) δ 7.63-7.28 (comp, 6H), 7.19 (d, J=8.4 Hz, 1H), 6.01 (d, J=4.0 Hz, 0.65H), 5.19 (d, J=4.0 Hz, 0.35H), 4.32 (dd, J=13.6, 6.4 Hz, 0.35H), 3.60 (dd, J=13.6, 6.4 Hz, 0.65H), 3.39 (s, 1H), 2.95-2.61 (comp, 3H), 2.55-2.22 (comp, 2H), 2.04-1.97 (m, 1H), 1.86-1.78 (comp, 2H), 1.74-1.66 (comp, 3H), 1.64-1.50 (comp, 3H), 1.46 (d, J=8.8 Hz, 9H). ¹³C NMR (126 MHz, as a mixture of rotamers) δ 172.4, 171.1, 154.7, 146.7, 144.4, 142.0, 139.2, 129.3 (q, J_{C-F}=32.8 Hz), 127.7, 127.3, 125.7 (q, J_{C-F}=3.8 Hz), 124.2 (q, J_{C-F}=272.8 Hz), 123.2, 122.7, 79.5, 54.6, 44.0, 43.3, 39.8, 38.9, 31.1, 29.7, 28.7, 28.4. HRMS (ESI) m/z calcd for C₂₉H₃₃F₃N₂NaO₃ (M+Na)⁺, 537.2335. found 537.2339.

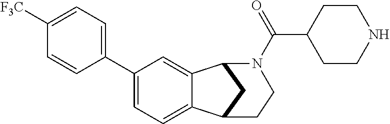

Piperidin-4-yl((1S,5R)-8-(4-(trifluoromethyl)phenyl)-1,3,4,5-tetrahydro-2H-1,5-methanobenzo[c]azepin-2-yl)methanone. ¹H NMR (400 MHz, as a mixture of rotamers) δ 7.72-7.62 (comp, 4H), 7.56-7.40 (comp, 2H), 7.38-7.31 (m, 1H), 6.00 (d, J=4.0 Hz, 0.65H), 5.19 (d, J=4.0 Hz, 0.35H), 4.30 (dd, J=14.0, 6.4 Hz, 0.35H), 3.37 (s, 1H), 3.27-3.07 (comp, 2H), 2.95-2.51 (comp, 3.65H), 2.25-2.30 (comp, 3H) 2.05-1.96 (m, 1H), 1.86-1.79 (comp, 2H), 1.77-1.66 (comp, 3H). ¹³C NMR (126 MHz, as a mixture of rotamers) δ 171.1, 171.0, 146.6, 146.4, 144.3, 141.7, 140.8, 139.4, 139.3, 129.3 (q, J_{C-F}=32.3 Hz), 128.3, 127.8, 127.4, 127.3, 125.7 (q, J_{C-F}=3.8 Hz), 124.2 (q, J_{C-F}=272.2 Hz), 123.4, 123.2, 122.8, 122.4, 59.1, 54.9, 43.8, 42.8, 42.6, 40.2, 39.6, 35.1, 31.1. HRMS (ESI) m/z calcd for C₂₄H₂₆F₃N₂O (M+H)⁺, 415.1992. found 415.2004.

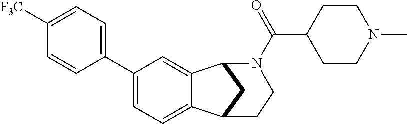

(1-Methylpiperidin-4-yl)((1R,5S)-8-(4-(trifluoromethyl)phenyl)-1,3,4,5-Tetrahydro-2H-1,5-methanobenzo[c]azepin-2-yl)methanone. ¹H NMR (500 MHz, as a mixture of rotamers) δ 7.74-7.62 (comp, 4H), 7.57-7.34 (comp, 3H), 5.96 (d, J=4.0 Hz, 0.6H), 5.10 (d, J=4.0 Hz, 0.4H), 4.27 (dd, J=14.5, 6.0 Hz, 0.4H), 3.93-3.47 (m, 2.6H), 3.46-3.18 (m, 5H), 3.76 (s, 3H), 2.42-2.28 (comp, 2H), 2.20-1.95 (comp, 3H), 1.92-1.80 (comp, 2H), 1.76-1.70 (m, 1H). ¹³C NMR (126 MHz, as a mixture of rotamers) δ 172.2, 172.0, 146.5, 146.3, 144.3, 141.7, 140.6, 139.5, 139.4, 129.3 (q, J_{C-F}=32.8 Hz), 128.4, 127.9, 127.4, 127.3, 125.8 (q, J_{C-F}=3.8 Hz), 124.2 (q, J_{C-F}=272.2 Hz), 123.6, 123.3, 122.7, 59.3, 54.7, 44.4, 43.8, 40.4, 39.7, 39.6, 36.5, 31.0, 29.7. HRMS (ESI) m/z calcd for C₂₅H₂₈F₃N₂O (M+H)⁺ 429.2148. found 429.2160.

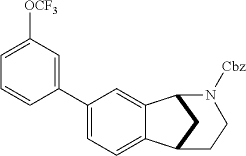

Benzyl 8-(3-(trifluoromethoxy)phenyl)-1,3,4,5-tetrahydro-2H-1,5-methanobenzo[c]azepine-2-carboxylate. ¹H NMR (400 MHz, as a mixture of rotamers) δ 7.52-7.11 (comp, 12H), 5.55 (br s, 0.5H), 5.43 (br s, 0.5H), 5.26-5.02 (comp, 2H), 3.91-3.72 (m, 1H), 3.29-3.16 (m, 1H), 2.56-2.34 (m, 1H), 2.27-2.11 (m, 1H), 2.03-1.91 (m, 1H), 1.87 (d, J=11.2 Hz, 1H), 1.65-1.52 (m, 1H). ¹³C NMR (126 MHz, as a mixture of rotamers) δ 154.9, 149.6 (q, J_{C-F}=2.1 Hz), 146.3, 143.2, 139.0, 130.0, 128.5, 128.0, 127.9, 127.4, 125.4, 123.1, 122.5, 120.5 (q, J_{C-F}=257.0 Hz), 119.7, 119.4, 67.0, 57.4, 43.6, 39.5, 38.6, 30.2. HRMS (ESI) m/z calcd for C₂₆H₂₂F₃NNaO₃ (M+Na)⁺, 476.1444. found 476.1449.

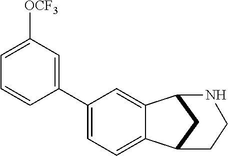

8-(3-(Trifluoromethoxy)phenyl)-2,3,4,5-tetrahydro-1H-1,5-methanobenzo[c]azepine. ¹H NMR (400 MHz) δ 7.55-7.39 (comp, 5H), 7.28 (d, J=7.6 Hz, 1H), 7.20-7.14 (m, 1H), 4.36 (d, J=4.4 Hz, 1H), 3.47-3.39 (m, 1H), 3.29-3.23 (m, 1H), 2.81 (dd, J=12.0, 6.0 Hz, 1H), 2.38 (td, J=12.4, 4.8 Hz, 1H), 2.30-2.22 (m, 1H), 2.08-2.01 (comp, 2H), 1.64-1.53 (m, 1H). ¹³C NMR (100 MHz) δ149.6 (q, J_{C-F}=2.5 Hz), 146.2, 143.5, 142.9, 138.8, 130.0, 127.0, 125.4, 122.8, 122.0, 120.5 (q, J_{C-F}=322.6 Hz), 119.6, 119.3, 58.7, 44.8, 39.8, 39.0, 30.6. HRMS (ESI) m/z calcd for C₁₈H₁₇F₃NO (M+H)⁺, 320.1257. found 320.1266.

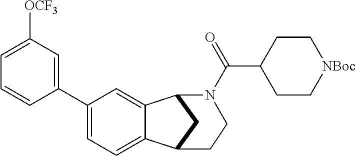

tert-Butyl 4-(8-(3-(trifluoromethoxy)phenyl)-2,3,4,5-tetrahydro-1H-1,5-methanobenzo[c]azepine-2-carbonyl)piperidine-1-carboxylate. ¹H NMR (500 MHz, as a mixture of rotamers) δ 7.53-7.38 (comp, 5H), 7.33 (d, J=8.0 Hz, 1H), 7.19 (d, J=8.0 Hz, 1H), 6.00 (br s, 0.5H), 4.20-4.05 (comp, 2H), 3.61 (br s, 0.5H), 3.42-3.35 (m, 1H), 2.95-2.25 (comp, 5H), 2.05-1.99 (m, 1H), 1.89-1.64 (comp, 7H), 1.46 (s, 9H). ¹³C NMR (126 MHz, as a mixture of rotamers) δ 172.4, 154.7, 149.7 (q, J_{C-F}=1.3 Hz), 146.5, 143.1, 142.0, 139.2, 130.1, 127.6, 125.4, 123.2, 122.6, 120.5 (q, J_{C-F}=257.0 Hz), 119.6, 119.5, 79.6, 54.8, 44.0, 43.3, 39.8, 38.9, 31.1, 29.7, 28.7, 28.4. HRMS (ESI) m/z calcd for C₂₉H₃₃F₃N₂NaO₄ (M+Na)⁺, 553.2285. found 553.2288.

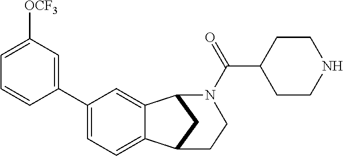

Piperidin-4-yl((1R,5S)-8-(3-(trifluoromethoxy)phenyl)-1,3,4,5-tetrahydro-2H-1,5-methanobenzo[c]azepin-2-yl)methanone. ¹H NMR (400 MHz, as a mixture of rotamers) δ 7.53-7.13 (comp, 7H), 5.99 (d, J=4.0 Hz, 0.65H), 5.20 (d, J=4.0 Hz, 0.35H), 4.30 (dd, J=14.0, 6.4 Hz, 0.35H), 3.61 (dd, J=14.0, 6.4 Hz, 0.65H), 3.41-3.33 (m, 1H), 3.30-3.09 (comp, 2H), 2.99-2.45 (comp, 5H), 2.34-2.22 (m, 1H), 2.05-1.94 (m, 1H), 1.93-1.56 (comp, 6H). ¹³C NMR (126 MHz, as a mixture of rotamers) δ 172.7, 172.4, 149.6, 146.5, 146.4, 143.10, 143.05, 142.1, 139.2, 139.0, 130.1, 130.0, 128.0, 127.4, 125.5, 125.3, 123.3, 123.0, 122.7, 122.2, 120.5 (q, J_{C-F}=253.3 Hz), 119.7, 119.6, 119.4, 58.7, 54.6, 45.5, 45.4, 45.3, 44.4, 43.9, 39.9, 39.8, 39.7, 38.6, 36.4, 31.2, 30.5. HRMS (ESI) m/z calcd for C₂₄H₂₆F₃N₂O₂ (M+H)⁺, 431.1941. found 431.1949.

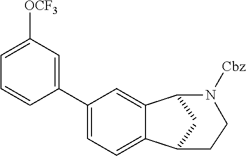

Benzyl 8-(3-(trifluoromethoxy)phenyl)-1,3,4,5-tetrahydro-2H-1,5-methanobenzo[c]azepine-2-carboxylate. ¹H NMR (400 MHz, as a mixture of rotamers) δ 7.52-7.11 (comp, 12H), 5.55 (br s, 0.5H), 5.43 (br s, 0.5H), 5.26-5.02 (comp, 2H), 3.91-3.72 (m, 1H), 3.29-3.16 (m, 1H), 2.56-2.34 (m, 1H), 2.27-2.11 (m, 1H), 2.03-1.91 (m, 1H), 1.87 (d, J=11.2 Hz, 1H), 1.65-1.52 (m, 1H). ¹³C NMR (126 MHz, as a mixture of rotamers) δ 154.9, 149.6 (q, J_{C-F}=2.1 Hz), 146.3, 143.2, 139.0, 130.0, 128.5, 128.0, 127.9, 127.4, 125.4, 123.1, 122.5, 120.5 (q, J_{C-F}=257.0 Hz), 119.7, 119.4, 67.0, 57.4, 43.6, 39.5, 38.6, 30.2. HRMS (ESI) m/z calcd for C₂₆H₂₂F₃NNaO₃ (M+Na)⁺, 476.1444. found 476.1449.

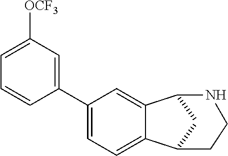

8-(3-(Trifluoromethoxy)phenyl)-2,3,4,5-tetrahydro-1H-1,5-methanobenzo[c]azepine. ¹H NMR (400 MHz) δ 7.55-7.39 (comp, 5H), 7.28 (d, J=7.6 Hz, 1H), 7.20-7.14 (m, 1H), 4.36 (d, J=4.4 Hz, 1H), 3.47-3.39 (m, 1H), 3.29-3.23 (m, 1H), 2.81 (dd, J=12.0, 6.0 Hz, 1H), 2.38 (td, J=12.4, 4.8 Hz, 1H), 2.30-2.22 (m, 1H), 2.08-2.01 (comp, 2H), 1.64-1.53 (m, 1H). ¹³C NMR (100 MHz) δ149.6 (q, J_{C-F}=2.5 Hz), 146.2, 143.5, 142.9, 138.8, 130.0, 127.0, 125.4, 122.8, 122.0, 120.5 (q, J_{C-F}=322.6 Hz), 119.6, 119.3, 58.7, 44.8, 39.8, 39.0, 30.6. HRMS (ESI) m/z calcd for C₁₈H₁₇F₃NO (M+H)⁺, 320.1257. found 320.1266.

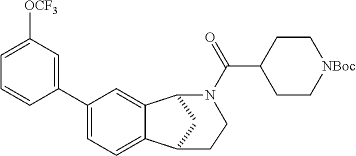

tert-Butyl 4-(8-(3-(trifluoromethoxy)phenyl)-2,3,4,5-tetrahydro-1H-1,5-methanobenzo[c]azepine-2-carbonyl)piperidine-1-carboxylate. ¹H NMR (500 MHz, as a mixture of rotamers) δ 7.53-7.38 (comp, 5H), 7.33 (d, J=8.0 Hz, 1H), 7.19 (d, J=8.0 Hz, 1H), 6.00 (br s, 0.5H), 4.20-4.05 (comp, 2H), 3.61 (br s, 0.5H), 3.42-3.35 (m, 1H), 2.95-2.25 (comp, 5H), 2.05-1.99 (m, 1H), 1.89-1.64 (comp, 7H), 1.46 (s, 9H). ¹³C NMR (126 MHz, as a mixture of rotamers) δ 172.4, 154.7, 149.7 (q, J_{C-F}=1.3 Hz), 146.5, 143.1, 142.0, 139.2, 130.1, 127.6, 125.4, 123.2, 122.6, 120.5 (q, J_{C-F}=257.0 Hz), 119.6, 119.5, 79.6, 54.8, 44.0, 43.3, 39.8, 38.9, 31.1, 29.7, 28.7, 28.4. HRMS (ESI) m/z calcd for C₂₉H₃₃F₃N₂NaO₄ (M+Na)⁺, 553.2285. found 553.2288.

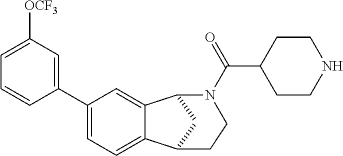

Piperidin-4-yl((1S,5R)-8-(3-(trifluoromethoxy)phenyl)-1,3,4,5-tetrahydro-2H-1,5-methanobenzo[c]azepin-2-yl)methanone. ¹H NMR (400 MHz, as a mixture of rotamers) δ 7.53-7.13 (comp, 7H), 5.99 (d, J=4.0 Hz, 0.65H), 5.20 (d, J=4.0 Hz, 0.35H), 4.30 (dd, J=14.0, 6.4 Hz, 0.35H), 3.61 (dd, J=14.0, 6.4 Hz, 0.65H), 3.41-3.33 (m, 1H), 3.30-3.09 (comp, 2H), 2.99-2.45 (comp, 5H), 2.34-2.22 (m, 1H), 2.05-1.94 (m, 1H), 1.93-1.56 (comp, 6H). ¹³C NMR (126 MHz, as a mixture of rotamers) δ 172.7, 172.4, 149.6, 146.5, 146.4, 143.10, 143.05, 142.1, 139.2, 139.0, 130.1, 130.0, 128.0, 127.4, 125.5, 125.3, 123.3, 123.0, 122.7, 122.2, 120.5 (q, J_{C-F}=253.3 Hz), 119.7, 119.6, 119.4, 58.7, 54.6, 45.5, 45.4, 45.3, 44.4, 43.9, 39.9, 39.8, 39.7, 38.6, 36.4, 31.2, 30.5. HRMS (ESI) m/z calcd for C₂₄H₂₆F₃N₂O₂ (M+H)⁺, 431.1941. found 431.1949.

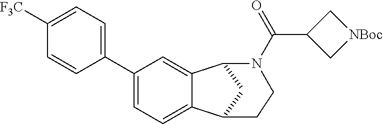

tert-Butyl 3-((1S,5R)-8-(4-(trifluoromethyl)phenyl)-2,3,4,5-tetrahydro-1H-1,5-methanobenzo[c]azepine-2-carbonyl) azetidine-1-carboxylate. ¹H NMR (500 MHz, as a mixture of rotamers) δ 7.72-7.61 (comp, 4H), 7.58-7.31 (comp, 3H), 5.98 (d, J=4.0 Hz, 0.65H), 4.73 (d, J=4.0 Hz, 0.35H), 4.30 (dd, J=14.0, 6.5 Hz, 0.35H), 4.26-3.91 (comp, 4H), 3.75-3.31 (comp, 2H), 3.17 (dd, J=14.0, 6.5 Hz, 0.65H), 2.72-2.25 (comp, 2H), 2.04-1.95 (m, 1H), 1.91-1.83 (m, 1H), 1.80-1.65 (m, 1H), 1.46-1.40 (comp, 9H). ¹³C NMR (126 MHz, as a mixture of rotamers) δ 169.2, 168.6, 156.3, 156.2, 146.5, 146.2, 144.3, 141.8, 140.7, 139.4, 139.3, 129.3 (q, J_{C-F}=32.8 Hz), 128.3, 127.7, 127.4, 127.3, 125.7 (q, J_{C-F}=3.8 Hz), 124.2 (q, J_{C-F}=272.2 Hz), 123.5, 123.2, 122.9, 122.2, 79.8, 79.7, 58.9, 55.1, 44.2, 43.7, 39.8, 39.7, 31.4, 30.8, 28.3. HRMS (ESI) m/z calcd for C₂₇H₂₉F₃N₂NaO₃ (M+Na)⁺, 509.2022. found 509.2026.

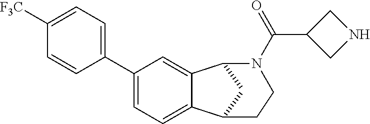

Azetidin-3-yl((1S,5R)-8-(4-(trifluoromethyl)phenyl)-1,3,4,5-tetrahydro-2H-1,5-methanobenzo[c]azepin-2-yl)methanone. ¹H NMR (500 MHz, as a mixture of rotamers) δ 7.74-7.60 (comp, 4H), 7.57-7.31 (comp, 3H), 5.97 (d, J=4.0 Hz, 0.65H), 4.84 (d, J=4.0 Hz, 0.35H), 4.27 (dd, J=14.0, 6.5 Hz, 0.35H), 3.79-3.61 (comp, 3.65H), 3.42-3.35 (m, 1H), 3.32-3.21 (m, 1H), 2.67-2.25 (comp, 3H), 2.05-1.93 (m, 1H), 1.90-1.81 (m, 1H), 1.75-1.61 (m, 1H).

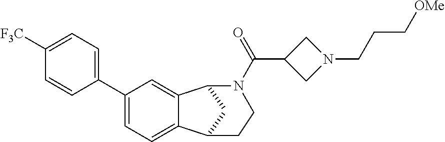

(1-(3-Methoxypropyl) azetidin-3-yl)((1S,5R)-8-(4-(trifluoromethyl)phenyl)-1,3,4,5-tetrahydro-2H-1,5-methanobenzo[c]azepin-2-yl)methanone. ¹H NMR (500 MHz, as a mixture of rotamers) δ 7.72-7.60 (comp, 4H), 7.55-7.31 (comp, 3H), 5.94 (d, J=4.0 Hz, 0.65H), 4.87 (d, J=4.0 Hz, 0.35H), 4.25 (dd, J=14.0, 6.5 Hz, 0.35H), 4.15-3.75 (comp, 2.65H), 3.68-3.51 (comp, 2H), 3.47-3.27 (comp, 7H), 2.86-2.72 (comp, 2H), 2.68-2.22 (comp, 2H), 2.06-1.92 (m, 1H), 1.87-1.81 (m, 1H), 1.80-1.64 (comp, 3H). ¹³C NMR (126 MHz, as a mixture of rotamers) δ 168.6, 168.1, 146.5, 146.2, 144.3, 141.6, 140.6, 139.4, 139.3, 129.3 (q, J_{C-F}=32.8 Hz), 128.3, 127.8, 127.4, 127.3, 125.7 (q, J_{C-F}=3.8 Hz), 124.2 (q, J_{C-F}=272.2 Hz), 123.5, 123.2, 122.8, 122.2, 69.84, 69.80, 58.9, 58.64, 58.61, 56.7, 56.5, 56.4, 56.2, 55.12, 55.08, 55.0, 44.2, 43.7, 39.8, 39.7, 39.6, 36.8, 32.64, 32.56, 30.8, 26.6, 26.5. HRMS (ESI) m/z calcd for C₂₆H₃₀F₃N₂O₂ (M+H)⁺, 459.2254. found 459.2256.

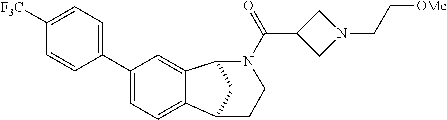

(1-(2-Methoxyethyl) azetidin-3-yl)((1S,5R)-8-(4-(trifluoromethyl)phenyl)-1,3,4,5-tetrahydro-2H-1,5-methanobenzo[c]azepin-2-yl)methanone. ¹H NMR (500 MHz, as a mixture of rotamers) δ 7.72-7.60 (comp, 4H), 7.57-7.30 (comp, 3H), 5.95 (d, J=4.0 Hz, 0.65H), 4.85 (d, J=4.0 Hz, 0.35H), 4.26 (dd, J=14.0, 6.5 Hz, 0.35H), 3.96-3.58 (comp, 2.65H), 3.52-3.43 (comp, 2H), 3.42-3.34 (comp, 4H), 3.31 (s, 3H), 2.80-2.23 (comp, 4H), 2.05-1.97 (m, 1H), 1.89-1.81 (m, 1H), 1.74-1.62 (m, 1H). ¹³C NMR (126 MHz, as a mixture of rotamers) δ 169.6, 169.1, 146.6, 146.3, 144.4, 141.9, 140.9, 139.3, 139.2, 129.3 (q, J_{C-F}=32.8 Hz), 128.2, 127.6, 127.4, 127.3, 125.7 (q, J_{C-F}=3.8 Hz), 124.2 (q, J_{C-F}=272.2 Hz), 123.5, 123.2, 122.8, 122.2, 70.3, 59.0, 58.9, 58.8, 58.0, 57.6, 57.4, 57.3, 57.1, 54.9, 44.2, 43.8, 39.8, 39.7, 39.6, 36.6, 34.0, 33.9, 30.9. HRMS (ESI) m/z calcd for C₂₅H₂₈F₃N₂O₂ (M+H)⁺, 445.2097. found 445.2095.

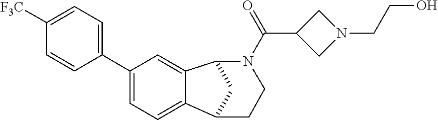

(1-(2-Hydroxyethyl) azetidin-3-yl)((1S,5R)-8-(4-(trifluoromethyl)phenyl)-1,3,4,5-tetrahydro-2H-1,5-methanobenzo[c]azepin-2-yl)methanone. ¹H NMR (500 MHz, as a mixture of rotamers) δ 7.72-7.60 (comp, 4H), 7.56-7.31 (comp, 3H), 5.94 (d, J=4.0 Hz, 0.65H), 4.87 (d, J=4.0 Hz, 0.35H), 4.26 (dd, J=14.0, 6.5 Hz, 0.35H), 4.15-4.05 (m, 1H), 3.99-3.90 (m, 1H), 3.83-3.58 (comp, 6H), 3.42-3.37 (m, 1H), 3.30 (dd, J=14.0, 6.5 Hz, 0.65H), 2.97-2.86 (comp, 2H), 2.69-2.60 (m, 0.65H), 2.35-2.25 (comp, 1.35H), 2.04-1.96 (m, 1H), 1.88-1.83 (m, 1H), 1.76-1.64 (m, 1H). ¹³C NMR (126 MHz, as a mixture of rotamers) δ 168.5, 168.1, 146.5, 146.2, 144.34, 144.29, 141.6, 140.6, 139.4, 139.3, 129.3 (q, J_{C-F}=32.8 Hz), 128.3, 127.8, 127.4, 127.3, 125.7 (q, J_{C-F}=3.8 Hz), 124.2 (q, J_{C-F}=272.2 Hz), 123.5, 123.3, 122.8, 122.3, 59.7, 59.6, 59.0, 58.4, 58.3, 56.9, 56.8, 56.7, 56.5, 55.2, 53.4, 44.3, 43.7, 39.8, 39.7, 39.6, 36.8, 33.0, 32.9, 30.8. HRMS (ESI) m/z calcd for C₂₄H₂₆F₃N₂O₂ (M+H)⁺, 431.1941. found 431.1946.

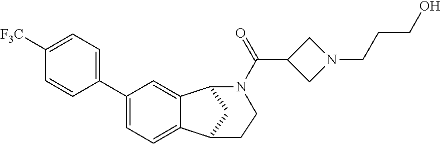

(1-(3-Hydroxypropyl) azetidin-3-yl)((1S,5R)-8-(4-(trifluoromethyl)phenyl)-1,3,4,5-tetrahydro-2H-1,5-methanobenzo[c]azepin-2-yl)methanone. ¹H NMR (500 MHz, as a mixture of rotamers) δ 7.72-7.60 (comp, 4H), 7.56-7.31 (comp, 3H), 5.93 (d, J=4.0 Hz, 0.65H), 4.89 (d, J=4.0 Hz, 0.35H), 4.34-3.99 (comp, 3H), 3.83-3.60 (comp, 5H), 3.44-3.28 (comp, 2H), 3.15-2.60 (comp, 3H), 2.36-2.28 (m, 1H), 1.85 (d, J=11.0 Hz, 1H), 1.81-1.67 (comp, 3H). ¹³C NMR (126 MHz, as a mixture of rotamers) δ 167.8, 167.3, 146.4, 146.1, 144.3, 144.2, 141.4, 140.4, 139.5, 139.4, 129.4 (q, J_{C-F}=32.8 Hz), 128.4, 128.0, 127.4, 127.3, 125.7 (q, J_{C-F}=3.8 Hz), 124.2 (q, J_{C-F}=272.2 Hz), 123.6, 123.3, 122.8, 122.2, 60.9, 60.8, 59.1, 56.4, 56.2, 56.1, 56.0, 55.4, 53.4, 45.9, 44.3, 43.8, 39.9, 39.7, 39.6, 37.0, 32.0, 31.9, 30.8, 27.43, 27.36. HRMS (ESI) m/z calcd for C₂₅H₂₈F₃N₂O₂ (M+H)⁺, 445.2097. found 445.2107.

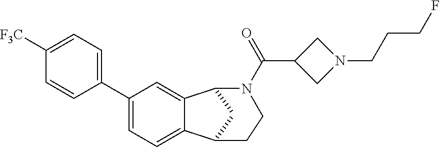

(1-(3-Fluoropropyl) azetidin-3-yl)((1S,5R)-8-(4-(trifluoromethyl)phenyl)-1,3,4,5-tetrahydro-2H-1,5-methanobenzo[c]azepin-2-yl)methanone. ¹H NMR (500 MHz, as a mixture of rotamers) δ 7.72-7.61 (comp, 4H), 7.58-7.30 (comp, 3H), 5.96 (d, J=4.0 Hz, 0.65H), 4.86 (d, J=4.0 Hz, 0.35H), 4.58-4.22 (comp, 2H), 3.87-3.20 (comp, 7H), 2.73-2.22 (comp, 4H), 2.04-1.96 (m, 1H), 1.89-1.65 (comp, 4H). ¹³C NMR (126 MHz, as a mixture of rotamers) & 169.6, 169.1, 146.6, 146.3, 144.4, 141.9, 140.9, 139.4, 139.2, 129.3 (q, J_{C-F}=32.8 Hz), 128.2, 127.7, 127.4, 127.3, 125.7 (q, J_{C-F}=3.8 Hz), 124.2 (q, J_{C-F}=272.2 Hz), 123.5, 123.2, 122.8, 122.2, 81.9 (d, J=168.8 Hz), 58.9, 57.0, 56.8, 56.7, 56.5, 54.9, 54.8 (d, J=5.4 Hz), 53.4, 44.2, 43.8, 39.8, 39.68, 39.67, 36.6, 33.4, 33.3, 30.9, 28.2 (d, J=20.2 Hz). HRMS (ESI) m/z calcd for C₂₅H₂₇F₄N₂O (M+H)⁺, 447.2054. found 447.2059.

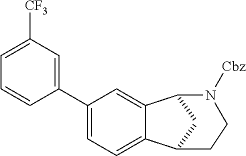

Benzyl (1S,5R)-8-(3-(trifluoromethyl)phenyl)-1,3,4,5-tetrahydro-2H-1,5-methanobenzo[c]azepine-2-carboxylate. ¹H NMR (500 MHz, as a mixture of rotamers) δ 7.84-7.28 (comp, 11H), 5.60 (br s, 0.5H), 5.48 (br s, 0.5H), 5.28-5.06 (comp, 2H), 3.94-3.78 (m, 1H), 3.37-3.22 (m, 1H), 2.57-2.40 (m, 1H), 2.36-2.20 (m, 1H), 2.13-1.98 (m, 1H), 1.92 (d, J=11.0 Hz, 1H), 1.74-1.59 (m, 1H); ¹³C NMR (126 MHz, as a mixture of rotamers) δ 155.0, 154.8, 146.3, 142.1, 141.9, 139.1, 136.9, 136.8, 131.2 (q, J_{C-F}=32.8 Hz), 130.4, 129.2, 128.5, 127.9, 127.6, 127.4, 125.3, 123.8 (m), 123.2, 122.7, 122.0 (q, J_{C-F}=272.2 Hz), 122.5, 67.1, 57.6, 57.3, 43.7, 39.5, 38.6, 30.2. HRMS (ESI) m/z calcd for C₂₆H₂₂F₃NNaO₂ (M+Na)⁺, 460.1495. found 460.1501.

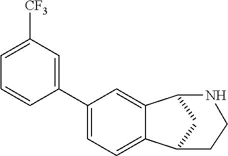

(1S,5R)-8-(3-(Trifluoromethyl)phenyl)-2,3,4,5-tetrahydro-1H-1,5-methanobenzo[c]azepine. ¹H NMR (500 MHz) δ 7.84 (s, 1H), 7.76 (d, J=8.0 Hz, 1H), 7.61-7.43 (comp, 4H), 7.28 (d, J=8.0 Hz, 1H), 4.31 (d, J=4.0 Hz, 1H), 3.26-3.21 (m, 1H), 2.92 (dd, J=6.0, 4.0 Hz, 1H), 2.36 (td, J=12.0, 4.0 Hz, 1H), 2.28-2.22 (m, 1H), 2.03-1.96 (comp, 2H), 1.61-1.54 (m, 1H); ¹³C NMR (126 MHz) δ 146.3, 143.5, 142.2, 138.8, 131.0 (q, J_{C-F}=31.5 Hz), 130.2, 129.1, 126.8, 123.8 (q, J_{C-F}=3.8 Hz), 123.6 (q, J_{C-F}=3.8 Hz), 123.4 (q, J_{C-F}=272.2 Hz), 122.8, 121.8, 58.6, 45.1, 39.9, 38.9, 30.9. HRMS (ESI) m/z calcd for C₁₈H₁₇F₃N (M+H)⁺, 304.1308. found 304.1312.

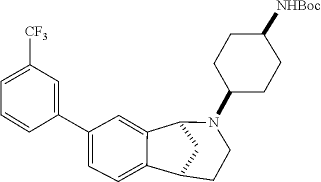

tert-Butyl (4-((1S,5R)-8-(3-(trifluoromethyl)phenyl)-1,3,4,5-tetrahydro-2H-1,5-methanobenzo[c]azepin-2-yl)cyclohexyl)carbamate. ¹H NMR (500 MHz) δ 7.81 (s, 1H), 7.75 (d, J=7.5 Hz, 1H), 7.61-7.52 (comp, 2H), 7.44 (d, J=7.5, 1H), 7.31-7.27 (comp, 2H), 4.769 (br s, 1H), 4.39 (d, J=3.0 Hz, 1H), 3.68 (br s, 1H), 3.16 (s, 1H), 2.83 (q, J=5.0 Hz, 1H), 2.30-2.21 (m, 1H), 2.14-2.07 (m, 1H), 2.03-1.89 (comp, 3H), 1.87-1.75 (comp, 2H), 1.72-1.44 (comp, 7H), 1.44 (s, 9H); ¹³C NMR (126 MHz) δ 155.2, 146.8, 142.4, 140.5, 138.1, 131.1 (q, J_{C-F}=32.2 Hz), 130.5, 129.2, 126.7, 124.2 (q, J_{C-F}=272.2 Hz), 123.9 (q, J_{C-F}=3.8 Hz), 123.6 (q, J_{C-F}=3.8 Hz), 122.9, 122.7, 78.9, 58.7, 57.4, 46.7, 44.5, 43.5, 39.6, 30.6, 29.7, 28.5, 28.4, 26.5, 25.9; HRMS (ESI) m/z calcd for C₂₉H₃₆F₃N₂O₂ (M+H)⁺, 501.2723. found 501.2721.

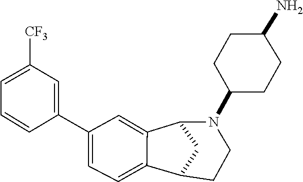

4-((1S,5R)-8-(3-(Trifluoromethyl)phenyl)-1,3,4,5-tetrahydro-2H-1,5-methanobenzo[c]azepin-2-yl)cyclohexan-1-amine. ¹H NMR (500 MHz) δ 7.81 (s, 1H), 7.75 (d, J=7.5 Hz, 1H), 7.61-7.51 (comp, 2H), 7.44 (dd, J=7.5, 1.5 Hz, 1H), 7.32-7.27 (comp, 2H), 4.39 (d, J=5.0 Hz, 1H), 3.15 (s, 1H), 2.91 (br s, 1H), 2.84 (q, J=5.0 Hz, 1H), 2.29-2.22 (m, 1H), 2.14-2.08 (m, 1H), 2.06-1.97 (comp, 2H), 1.95 (d, J=10.5 Hz, 1H), 1.81-1.40 (comp, 11H). ¹³C NMR (126 MHz) δ 146.9, 142.5, 140.8, 138.0, 131.1 (q, J_{C-F}=32.2 Hz), 130.5, 129.1, 126.6, 124.2 (q, J_{C-F}=272.2 Hz), 123.9 (q, J_{C-F}=3.8 Hz), 123.6 (q, J_{C-F}=3.8 Hz), 122.8, 122.7, 58.7, 57.5, 48.0, 44.5, 43.6, 39.7, 30.6, 26.4, 25.9. HRMS (ESI) m/z calcd for C₂₄H₂₈F₃N₂ (M+H)⁺, 401.2199. found 401.2193.

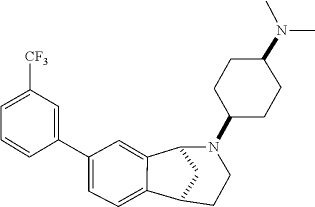

N,N-Dimethyl-4-((1S,5R)-8-(3-(trifluoromethyl)phenyl)-1,3,4,5-tetrahydro-2H-1,5-methanobenzo[c]azepin-2-yl)cyclohexan-1-amine. ¹H NMR (500 MHz) δ 7.81 (s, 1H), 7.75 (d, J=7.5 Hz, 1H), 7.60-7.52 (comp, 2H), 7.44-7.41 (m, 1H), 7.30-7.27 (comp, 2H), 4.32 (d, J=5.0 Hz, 1H), 3.14 (s, 1H), 3.06 (br s, 1H), 2.78-2.75 (m, 1H), 2.36 (s, 6H), 2.28-2.25 (m, 1H), 2.18-2.15 (comp, 2H), 2.12-2.09 (m, 1H), 1.85 (d, J=10.5 Hz, 1H), 1.68-1.40 (comp, 9H). ¹³C NMR (126 MHz) δ 146.9, 142.5, 140.8, 138.0, 131.1 (q, J_{C-F}=32.2 Hz), 130.5, 129.1, 126.6, 124.2 (q, J_{C-F}=272.2 Hz), 123.9 (q, J_{C-F}=3.8 Hz), 123.6 (q, J_{C-F}=3.8 Hz), 122.8, 122.7, 63.2, 58.5, 55.6, 44.5, 43.2, 41.8, 39.5, 30.6, 27.4, 26.9. HRMS (ESI) m/z calcd for C₂₆H₃₂F₃N₂ (M+H)⁺, 429.2512. found 429.2518.

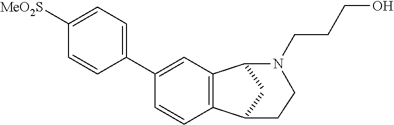

3-((1S,5R)-8-(4-(Methylsulfonyl)phenyl)-1,3,4,5-tetrahydro-2H-1,5-methanobenzo[c]azepin-2-yl) propan-1-ol. ¹H NMR (500 MHz, CDCl₃) δ 8.02 (d, J=8.0 Hz, 2H), 7.77 (d, J=8.4 Hz, 2H), 7.60 (d, J=7.6 Hz, 1H), 7.51 (s, 1H) 7.41 (d, J=7.7 Hz, 1H), 3.91-3.85 (comp, 2H), 3.40-3.15 (comp, 3H), 2.94-2.71 (comp, 3H), 2.51-2.21 (comp, 3H), 2.01-1.67 (comp, 6H). ¹³C NMR (126 MHz, CDCl₃) δ 171.3, 147.8, 146.3, 139.4, 138.6, 128.2, 128.2, 128.2, 128.1, 123.9, 63.8, 60.6, 47.3, 44.7, 38.9, 27.0, 21.2, 14.3. HRMS (ESI) m/z calcd for C₂₁H₂₅NO₃S (M+H)⁺, 372.1628. found 372.1629.

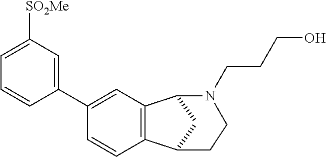

3-((1S,5R)-8-(3-(Methylsulfonyl)phenyl)-1,3,4,5-tetrahydro-2H-1,5-methanobenzo[c]azepin-2-yl) propan-1-ol. ¹H NMR (500 MHz, MeOD) δ 8.17 (br s, 1H), 7.98 (d, J=7.5 Hz, 1H), 7.93 (d, J=8.0 Hz, 1H), 7.71 (t, J=7.8 Hz, 1H), 7.60 (dd, J=7.6, 1.8 Hz, 1H), 7.55 (s, 1H), 7.37 (d, J=7.6 Hz, 1H), 4.14 (d, J=4.6 Hz, 1H), 3.67-3.62 (comp, 2H), 3.22 (s, 1H), 3.18 (s, 3H), 2.72 (dd, J=11.9, 5.7 Hz, 1H), 2.63-2.58 (m, 1H), 2.30-2.24 (comp, 2H), 2.07-2.03 (comp, 2H), 1.88-1.81 (m, 1H), 1.79-1.72 (m, 1H), 1.63-1.59 (m, 1H), 1.49 (td, J=12.2, 5.0 Hz, 1H). ¹³C NMR (126 MHz, MeOD) δ 173.0, 148.2, 144.3, 142.8, 140.7, 139.0, 133.3, 131.1, 128.4, 126.8, 126.8, 126.6, 124.3, 124.2, 64.6, 62.0, 61.5, 54.7, 44.9, 44.4, 40.8, 30.9, 30.4, 20.8, 14.4. HRMS (ESI) m/z calcd for C₂₁H₂₆NO₃S (M+H)⁺, 372.1628. found 372.1624.

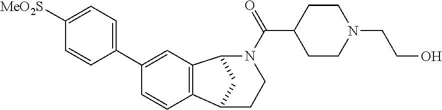

(1-(2-Hydroxyethyl)piperidin-4-yl)((1S,5R)-8-(4-(methylsulfonyl)phenyl)-1,3,4,5-tetrahydro-2H-1,5-methanobenzo[c]azepin-2-yl)methanone. ¹H NMR (500 MHz, MeOD) rotamers δ 8.03-8.01 (comp, 2H), 7.89 (t, J=8.5 Hz, 2H), 7.70-7.63 (comp, 2H), 7.47-7.45 (m, 1H), 5.92 (d, J=4.1 Hz, 0.6H), 5.47 (d, J=4.1 Hz, 0.4H), 4.23 (dd, J=13.9, 6.5 Hz, 0.6H), 3.85-3.78 (comp, 2.4H), 3.53-3.43 (m, 1H), 3.05-2.70 (comp, 9H), 2.39-2.29 (m, 1H), 2.26-2.08 (comp, 3H), 1.98-1.73 (comp, 6H). ¹³C NMR (126 MHz, MeOD) δ 174.0, 173.7, 172.9, 148.9, 148.6, 147.7, 147.7, 143.2, 142.7, 140.7, 140.6, 140.1, 140.0, 129.5, 129.3, 129.0, 129.0, 129.0, 128.9, 124.8, 124.6, 123.7, 123.6, 61.5, 60.2, 56.5, 49.8, 44.7, 44.4, 41.2, 41.1, 37.9, 32.0, 31.4, 20.8, 14.4, 9.2. HRMS (ESI) m/z calcd for C₂₆H₃₂N₂O₄S (M+H)⁺, 469.2156. found 469.2153.

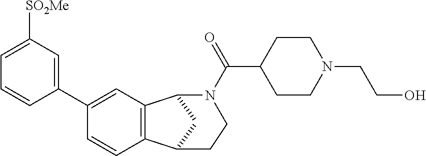

(1-(2-Hydroxyethyl)piperidin-4-yl)((1S,5R)-8-(3-(methylsulfonyl)phenyl)-1,3,4,5-tetrahydro-2H-1,5-methanobenzo[c]azepin-2-yl)methanone. ¹H NMR (500 MHz, MeOD) rotamers δ 8.17-8.15 (m, 1H), 7.98-7.91 (comp, 2H), 7.72-7.61 (comp, 3H), 7.44-7.42 (m, 1H), 5.90 (d, J=4.1 Hz, 0.6H), 5.44 (d, J=4.2 Hz, 0.4H), 4.22 (dd, J=13.9, 6.3 Hz, 0.4H), 3.79-3.69 (comp, 2.6H), 3.41 (s, 1H), 3.23-3.06 (comp, 6H), 2.73-2.62 (comp, 3H), 2.51-2.46 (m, 1H), 2.36-2.24 (comp, 2H), 2.12-2.05 (m, 1H), 1.97-1.62 (comp, 6H). ¹³C NMR (126 MHz, MeOD) rotamers δ 174.9, 174.6, 172.9, 148.6, 148.2, 143.8, 143.7, 143.3, 142.8, 142.8, 140.0, 139.9, 133.3, 133.2, 131.1, 129.2, 128.9, 127.0, 126.9, 126.6, 126.6, 124.7, 124.6, 123.5, 123.4, 61.5, 61.1, 61.1, 60.2, 59.2, 56.4, 54.2, 54.1, 54.1, 54.0, 45.4, 44.7, 44.3, 41.1, 41.1, 39.2, 38.9, 37.8, 32.1, 31.46, 29.2, 29.1, 29.0, 28.4, 20.8, 14.5. HRMS (ESI) m/z calcd for C₂₆H₃₂N₂O₄S (M+H)⁺, 469.2156. found 469.2152.

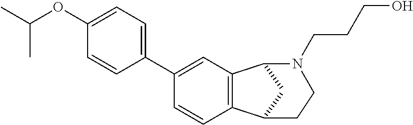

3-((1S,5R)-8-(4-Isopropoxyphenyl)-1,3,4,5-tetrahydro-2H-1,5-methanobenzo[c]azepin-2-yl) propan-1-ol. ¹H NMR (400 MHz, MeOD) δ 7.51-7.49 (comp, 2H), 7.43 (dd, J=7.6, 1.7 Hz, 1H), 7.38 (brs 1H), 7.23 (d, J=7.6 Hz, 1H), 6.95-6.93 (comp, 2H), 4.60 (hept, J=6.0 Hz, 1H), 4.03 (d, J=4.6 Hz, 1H), 3.65-3.61 (comp, 2H), 3.13 (brs, 1H), 2.64 (dd, J=11.6, 5.6 Hz, 1H), 2.59-2.52 (m, 1H), 2.27-2.18 (comp, 2H), 2.02-1.93 (comp, 3H), 1.87-1.69 (comp, 2H), 1.57-1.52 (m, 1H), 1.45 (td, J=12.0, 4.9 Hz, 1H), 1.32 (d, J=6.0 Hz, 6H). ¹³C NMR (101 MHz, MeOD) δ 158.70, 146.03, 140.68, 140.09, 135.08, 129.04, 127.53, 123.75, 123.63, 117.28, 71.04, 64.55, 62.05, 54.61, 47.99, 44.97, 40.67, 31.02, 30.46, 22.40. HRMS (ESI) m/z calcd for C₂₃H₂₉NO₂ (M+H)⁺, 352.2271. found 352.2269.

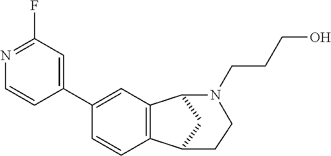

3-((1S,5R)-8-(2-Fluoropyridin-4-yl)-1,3,4,5-tetrahydro-2H-1,5-methanobenzo[c]azepin-2-yl) propan-1-ol. ¹H NMR (500 MHz, MeOD) δ 8.23 (d, J=5.3 Hz, 1H), 7.69 (dd, J=7.6, 1.8 Hz, 1H), 7.64-7.61 (comp, 2H), 7.41-7.38 (comp, 2H), 4.16 (d, J=4.6 Hz, 1H), 3.69-3.61 (comp, 2H), 3.23 (brs, 1H), 2.73 (dd, J=11.9, 5.7 Hz, 1H), 2.64-2.58 (m, 1H), 2.31-2.24 (comp, 2H), 2.08-2.02 (comp, 2H), 1.90-1.82 (m, 1H), 1.80-1.71 (m, 1H), 1.63-1.60 (m, 1H), 1.48 (td, J=12.2, 4.9 Hz, 1H). ¹³C NMR (126 MHz, MeOD) δ 172.9, 166.9, 165.0, 156.4, 156.3, 150.0, 148.7, 148.7, 140.8, 136.5, 136.4, 128.5, 124.5, 124.1, 120.9, 120.8, 108.0, 107.7, 64.4, 61.8, 61.5, 54.6, 47.9, 44.8, 40.8, 30.7, 30.4, 20.8, 14.4. ¹⁹F NMR (471 MHz, MeOD) δ −71.50. HRMS (ESI) m/z calcd for C₁₉H₂₁FN₂O (M+H)⁺, 313.1711. found 313.1711.

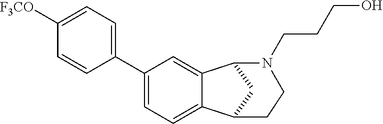

3-((1S,5R)-8-(4-(Trifluoromethoxy)phenyl)-1,3,4,5-tetrahydro-2H-1,5-methanobenzo[c]azepin-2-yl) propan-1-ol. ¹H NMR (400 MHz, CDCl₃) δ 7.59 (d, J=8.37 Hz, 2H), 7.55-7.50 (m, 1H), 7.45 (br s, 1H), 7.36 (br d, J=7.69 Hz, 1H), 7.30 (d, J=8.29 Hz, 2H), 4.45 (br s, 1H), 4.31-4.10 (m, 1H), 3.88 (br s, 2H), 3.38-3.27 (m, 1H), 3.23-3.07 (m, 1H), 3.04-2.85 (m, 1H), 2.42-2.20 (comp, 2H), 1.98-1.77 (comp 2H), 1.74-1.50 (m, 1H). ¹³C NMR (101 MHz, CDCl₃) δ 147.8, 145.4, 138.5, 127.5, 123.0, 122.6, 120.4, 118.3, 62.8, 46.3 37.9, 37.8, 35.9, 35.8, 28.7, 28.1, 25.9. LCMS (CI) m/z calcd for C₂₁H₂₂F₃NO₂ (M+H)⁺ 378.16. found 378.20.

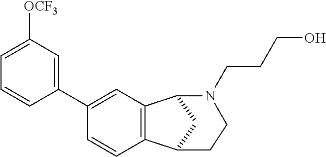

3-((1S,5R)-8-(3-(Trifluoromethoxy)phenyl)-1,3,4,5-tetrahydro-2H-1,5-methanobenzo[c]azepin-2-yl) propan-1-ol. ¹H NMR (400 MHz, CDCl₃) δ 7.56-7.27 (comp, 5H), 7.24-7.14 (comp, 2H), 4.41 (br s, 1H), 4.21 (d, J=4.6 Hz, 1H), 3.85 (q, J=5.7 Hz, 2H), 3.30 (s, 1H), 3.22-3.08 (m, 1H), 2.96 (m, 1H), 2.79 (m, 1H), 2.64 (m, 1H), 2.52 (s, 1H), 2.35 (s, 1H), 2.27 (m, 1H), 2.15-2.05 (m, 1H), 1.90-1.83 (m, 1H), 1.76 (d, J=4.8 Hz, 1H). ¹³C NMR (101 MHz, CDCl₃) δ 145.8, 144.1, 142.0, 137.8, 131.3, 129.2, 127.7, 124.5, 123.7, 123.0, 122.5, 118.7, 62.4, 61.2, 54.4, 45.9, 42.7, 37.9, 28.7, 27.8, 26.0. LCMS (CI) m/z calcd for C₂₁H₂₂F₃NO₂ (M+H)⁺ 378.16. found 378.2.

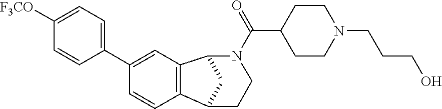

(1-(3-Hydroxypropyl)piperidin-4-yl)((1S,5R)-8-(4-(trifluoromethoxy)phenyl)-1,3,4,5-tetrahydro-2H-1,5-methanobenzo[c]azepin-2-yl)methanone. ¹H NMR (500 MHz, CDCl₃, as a mixture of rotamers) δ 7.60-7.53 (comp, 2H), 7.51-7.45 (comp, 2H), 7.40-7.31 (comp, 2H), 7.24-7.13 (m, 1H), 6.00-5.84 (m, 1H), 5.11 (br s, 1H), 4.33-4.20 (m, 1H), 3.82 (br s, 2H), 3.57-3.47 (m, 1H), 3.29-3.03 (comp, 5H), 2.84 (br s, 1H), 2.78-2.69 (m, 1H), 2.41-2.27 (comp, 3H), 2.26-2.19 (m, 1H), 2.19-2.09 (comp, 2H), 1.85-1.77 (m, 1H), 1.75-1.66 (comp, 2H). ¹³C NMR (126 MHz, CDCl₃, as a mixture of rotamers) δ 170.6, 147.6, 145.0, 140.5, 138.6, 138.4, 127.4, 126.6, 122.2, 121.6, 120.3, 58.4, 53.7, 43.5, 42.8, 39.4, 38.5, 35.5, 30.1, 29.4, 28.7, 25.6, 24.7. LCMS (CI) m/z calcd for C₂₇H₃₁F₃N₂O₃ (M+Na)⁺ 511.22. found 511.22.

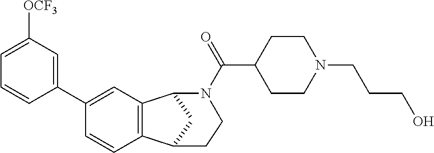

(1-(3-Hydroxypropyl)piperidin-4-yl)((1S,5R)-8-(3-(trifluoromethoxy)phenyl)-1,3,4,5-tetrahydro-2H-1,5-methanobenzo[c]azepin-2-yl)methanone. ¹H NMR (400 MHz, CDCl₃, as a mixture of rotamers) δ 7.52-7.45 (comp, 4H), 7.40-7.30 (comp, 3H), 7.24-7.14 (comp, 3H), 5.98 (d, J=4.16 Hz, 1H), 5.18-4.99 (m, 1H), 4.33-4.23 (m, 1H), 3.93-3.76 (comp, 5H), 3.60-3.45 (comp, 3H), 3.40-3.29 (comp, 2H), 3.26-3.14 (m, 1H), 2.76-2.64 (m, 1H), 2.36-2.16 (comp, 3H). ¹³C NMR (126 MHz, CDCl₃) δ 173.4, 148.7, 142.0, 129.9, 129.2, 127.8, 126.8, 124.4, 122.3, 118.7, 118.6, 63.3, 53.7, 49.9, 45.1, 42.9, 38.5, 30.9, 28.7, 28.4, 21.7, 13.1. LCMS (CI) m/z calcd for C₂₇H₃₂F₃N₂O₃ (M+H)⁺ 489.23. found 489.23.

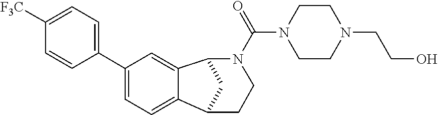

(4-(2-Hydroxyethyl)piperazin-1-yl)((1S,5R)-8-(4-(trifluoromethyl)phenyl)-1,3,4,5-tetrahydro-2H-1,5-methanobenzo[c]azepin-2-yl)methanone. ¹H NMR (500 MHz, MeOD) δ 7.84 (d, J=8.0 Hz, 2H), 7.79 (s, 1H), 7.73 (d, J=8.1 Hz, 2H), 7.63 (d, J=7.9 Hz, 1H), 7.54 (d, J=7.9 Hz, 1H), 6.40 (s, 1H), 3.85 (t, J=5.1 Hz, 2H), 3.71-3.59 (comp, 4H), 3.52 (t, J=7.4 Hz, 2H), 3.45 (s, 2H), 3.26-3.16 (comp, 7H), 2.83 (t, J=7.5 Hz, 2H). ¹³C NMR (126 MHz, MeOD) δ 159.4, 146.8, 146.8, 142.9, 137.4, 131.5, 129.8 (q, J_{C-F}=32.1 Hz), 128.5, 126.7 (q, J_{C-F}=3.8 Hz), 126.5, 125.9 (q, J_{C-F}=271.5 Hz), 123.7, 120.5, 59.8, 56.6, 53.0, 47.9, 42.3, 41.1, 38.9, 29.3. HRMS (ESI) m/z calcd for C₂₅H₂₉F₃N₃O₂ (M+H)⁺ 460.2206. found 460.2210.

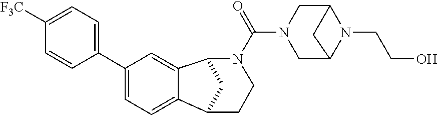

(6-(2-Hydroxyethyl)-3,6-diazabicyclo[3.1.1]heptan-3-yl)((1S,5R)-8-(4-(trifluoromethyl)phenyl)-1,3,4,5-tetrahydro-2H-1,5-methanobenzo[c]azepin-2-yl)methanone. ¹H NMR (500 MHz, CD₂Cl₂) 7.79-7.73 (comp, 3H), 7.70 (d, J=8.2 Hz, 2H), 7.58 (d, J=7.9 Hz, 1H), 7.53 (d, J=8.1 Hz, 1H), 6.39 (s, 1H), 4.76-4.57 (m, 1H), 3.83-3.77 (m, 1H), 3.74-3.66 (comp, 2H), 3.63-3.56 (comp, 5H), 3.56-3.51 (comp, 3H), 3.49-3.42 (comp, 2H), 3.39-3.27 (comp, 2H), 2.88-2.83 (comp, 2H), 2.60-2.55 (comp, 2H). ¹³C NMR (126 MHz, CD₂Cl₂) 158.1, 145.9, 145.9, 142.2, 136.7, 131.0, 129.1 (q, J_{C-F}=32.8 Hz), 127.80, 126.1 (q, J_{C-F}=3.8 Hz), 125.9, 125.0 (q, J_{C-F}=272.2 Hz, note: 1 peak was buried in 126.1 ppm), 123.1, 119.9, 59.7, 58.7, 56.4, 49.0, 47.7, 42.9, 39.9, 38.5, 29.1. ¹⁹F NMR (471 MHz, CD₂Cl₂) δ −62.6. HRMS (ESI) m/z calcd for C₂₆H₂₉F₃N₃O₂ (M+H)⁺ 472.2206. found 472.2209.

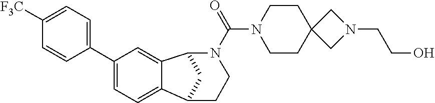

(2-(2-Hydroxyethyl)-2,7-diazaspiro[3.5]nonan-7-yl)((1S,5R)-8-(4-(trifluoromethyl)phenyl)-1,3,4,5-tetrahydro-2H-1,5-methanobenzo[c]azepin-2-yl)methanone. ¹H NMR (500 MHz, CD₂Cl₂) δ 7.78-7.73 (comp, 3H), 7.70 (d, J=8.2 Hz, 2H), 7.58 (d, J=7.8 Hz, 1H), 7.51 (d, J=7.9 Hz, 1H), 6.36 (s, 1H), 4.72 (br s, 1H), 3.60-3.54 (comp, 2H), 3.54-3.49 (comp, 2H), 3.46-3.43 (comp, 2H), 3.36-3.28 (comp, 4H), 3.27-3.21 (comp, 5H), 2.84-2.75 (comp, 4H), 1.77-1.71 (comp, 4H). ¹³C NMR (126 MHz, CD₂Cl₂) δ 157.7, 145.8, 145.8, 142.0, 136.5, 130.9, 128.9 (q, J_{C-F}=31.5 Hz), 127.7, 126.0 (q, J_{C-F}=3.8 Hz), 125.8, 124.9 (q, J_{C-F}=272.2 Hz), 123.0, 119.8, 64.1, 61.0, 58.8, 53.3, 41.5, 39.9, 38.4, 35.6, 35.2, 28.9. HRMS (ESI) m/z calcd for C₂₈H₃₃F₃N₃O₂ (M+H)⁺ 500.2519. found 500.2514.

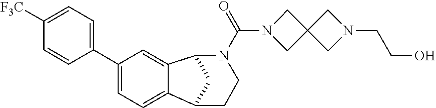

(6-(2-Hydroxyethyl)-2,6-diazaspiro[3.3]heptan-2-yl)((1S,5R)-8-(4-(trifluoromethyl)phenyl)-1,3,4,5-tetrahydro-2H-1,5-methanobenzo[c]azepin-2-yl)methanone. ¹H NMR (500 MHz, MeOD) δ 7.72 (d, J=8.0 Hz, 2H), 7.68 (s, 1H), 7.61 (d, J=8.1 Hz, 2H), 7.51 (d, J=7.9 Hz, 1H), 7.41 (d, J=7.8 Hz, 1H), 6.28 (s, 1H), 4.19 (s, 4H), 3.98 (s, 4H), 3.59 (t, J=4.6 Hz, 2H), 3.38-3.29 (comp, 5H), 3.14 (t, J=5.0 Hz, 2H), 2.67 (t, J=7.5 Hz, 2H). ¹³C NMR (126 MHz, MeOD) δ 161.8, 146.8, 146.7, 142.8, 137.4, 131.5, 129.8 (q, J_{C-F}=32.1 Hz), 128.5, 126.7 (q, J_{C-F}=3.8 Hz), 126.4, 125.9 (q, J_{C-F}=271.5 Hz), 123.6, 120.4, 64.4, 59.6, 57.8, 57.2, 53.6, 40.3, 38.8, 34.6, 29.5. HRMS (ESI) m/z calcd for C₂₆H₂₉F₃N₃O₂ (M+H)⁺ 472.2206. found 472.2206.

All of the compositions and methods disclosed and claimed herein can be made and executed without undue experimentation in light of the present disclosure. While the compositions and methods of this invention have been described in terms of certain embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Yousuf, M. S.; Sahn, J. J.; David, E. T.; Shiers, S.; Royer, D. M.; Garcia, C. D.; Zhang, J.; Hong, V. M.; Ahmad, A.; Kolber, B. J.; Liebl, D. J.; Martin, S. F.; Price, T. J. Validation of s2R/TMEM97 as a neuropathic pain target: Specificity, human expression and mechanism of action. bioRxiv, 2023.
A novel gene containing a trinucleotide repeat that is expanded and unstable on Huntington's disease chromosomes. The Huntington's Disease Collaborative Research Group. Cell 1993, 72, 971-983.
Walker, F. O. Huntington's Disease. Semin Neurol 2007, 27, 143-150.
Adam, O. R., and Jankovic, J. Symptomatic treatment of Huntington disease. Neurotherapeutics 2008, 5, 181-197.
Dash, D., and Mestre, T. A. Therapeutic Update on Huntington's Disease: Symptomatic Treatments and Emerging Disease-Modifying Therapies. Neurotherapeutics 2020, 17, 1645-1659.
Schmidt, H. R., and Kruse, A. C. The Molecular Function of sigma Receptors: Past, Present, and Future. Trends Pharmacol Sci 2019, 40, 636-654.
Smith, S. B. S., T (2017) Sigma Receptors: Their Role in Disease and as Therapeutic Agents, Springer, New York.
Pontisso, I., and Combettes, L. Role of Sigma-1 Receptor in Calcium Modulation: Possible Involvement in Cancer. Genes (Basel) 2021, 12.
Hayashi, T., and Su, T. P. Sigma-1 receptor chaperones at the ER-mitochondrion interface regulate Ca(2+) signaling and cell survival. Cell 2007, 131, 596-610.
Hayashi, T. Sigma-1 receptor: the novel intracellular target of neuropsychotherapeutic drugs. J Pharmacol Sci 2015, 127, 2-5.
Nguyen, L., Lucke-Wold, B. P., Mookerjee, S. A., Cavendish, J. Z., Robson, M. J., Scandinaro, A. L., and Matsumoto, R. R. Role of sigma-1 receptors in neurodegenerative diseases. J Pharmacol Sci 2015, 127, 17-29.
Nguyen, L., Lucke-Wold, B. P., Mookerjee, S., Kaushal, N., and Matsumoto, R. R. Sigma-1 Receptors and Neurodegenerative Diseases: Towards a Hypothesis of Sigma-1 Receptors as Amplifiers of Neurodegeneration and Neuroprotection. Adv Exp Med Biol 2017, 964, 133-152.
Ryskamp, D. A., Korban, S., Zhemkov, V., Kraskovskaya, N., and Bezprozvanny, I. Neuronal Sigma-1 Receptors: Signaling Functions and Protective Roles in Neurodegenerative Diseases. Front Neurosci 2019, 13, 862.
Hyrskyluoto, A., Pulli, I., Tornqvist, K., Ho, T. H., Korhonen, L., and Lindholm, D. Sigma-1 receptor agonist PRE084 is protective against mutant huntingtin-induced cell degeneration: involvement of calpastatin and the NF-kappaB pathway. Cell Death Dis 2013, 4, e646.
de Yebenes, J. G., Landwehrmeyer, B., Squitieri, F., Reilmann, R., Rosser, A., Barker, R. A., Saft, C., Magnet, M. K., Sword, A., Rembratt, A., Tedroff, J., and Mermai, H. D. s. i. Pridopidine for the treatment of motor function in patients with Huntington's disease (MermaiHD): a phase 3, randomised, double-blind, placebo-controlled trial. Lancet Neurol 2011, 10, 1049-1057.
Ryskamp, D., Wu, J., Geva, M., Kusko, R., Grossman, I., Hayden, M., and Bezprozvanny, I. The sigma-1 receptor mediates the beneficial effects of pridopidine in a mouse model of Huntington disease. Neurobiol Dis 2017, 97, 46-59.
Squitieri, F., Di Pardo, A., Favellato, M., Amico, E., Maglione, V., and Frati, L. Pridopidine, a dopamine stabilizer, improves motor performance and shows neuroprotective effects in Huntington disease R6/2 mouse model. J Cell Mol Med 2015, 19, 2540-2548.
Geva, M., Kusko, R., Soares, H., Fowler, K. D., Birnberg, T., Barash, S., Wagner, A. M., Fine, T., Lysaght, A., Weiner, B., Cha, Y., Kolitz, S., Towfic, F., Orbach, A., Laufer, R., Zeskind, B., Grossman, I., and Hayden, M. R. Pridopidine activates neuroprotective pathways impaired in Huntington Disease. Hum Mol Genet 2016, 25, 3975-3987.
Sahlholm, K., Valle-Leon, M., Taura, J., Fernandez-Duenas, V., and Ciruela, F. Effects of the Dopamine Stabilizer, Pridopidine, on Basal and Phencyclidine-Induced Locomotion: Role of Dopamine D2 and Sigma-1 Receptors. CNS Neurol Disord Drug Targets 2018, 17, 522-527.
Eddings, C. R., Arbez, N., Akimov, S., Geva, M., Hayden, M. R., and Ross, C. A. Pridopidine protects neurons from mutant-huntingtin toxicity via the sigma-1 receptor. Neurobiol Dis 2019, 129, 118-129.
Mach, R. H., Zeng, C., and Hawkins, W. G. The sigma2 receptor: a novel protein for the imaging and treatment of cancer. J Med Chem 2013, 56, 7137-7160.
Huang, Y. S., Lu, H. L., Zhang, L. J., and Wu, Z. Sigma-2 receptor ligands and their perspectives in cancer diagnosis and therapy. Med Res Rev 2014, 34, 532-566.
Qiu, G., Sun, W., Zou, Y., Cai, Z., Wang, P., Lin, X., Huang, J., Jiang, L., Ding, X., and Hu, G. RNA interference against TMEM97 inhibits cell proliferation, migration, and invasion in glioma cells. Tumour Biol 2015, 36, 8231-8238.
Zeng, C., and Mach, R. H. The Evolution of the Sigma-2 (sigma2) Receptor from Obscure Binding Site to Bona Fide Therapeutic Target. Adv Exp Med Biol 2017, 964, 49-61.
Izzo, N. J., Staniszewski, A., To, L., Fa, M., Teich, A. F., Saeed, F., Wostein, H., Walko, T., 3rd, Vaswani, A., Wardius, M., Syed, Z., Ravenscroft, J., Mozzoni, K., Silky, C., Rehak, C., Yurko, R., Finn, P., Look, G., Rishton, G., Safferstein, H., Miller, M., Johanson, C., Stopa, E., Windisch, M., Hutter-Paier, B., Shamloo, M., Arancio, O., LeVine, H., 3rd, and Catalano, S. M. Alzheimer's therapeutics targeting amyloid beta 1-42 oligomers I: Abeta 42 oligomer binding to specific neuronal receptors is displaced by drug candidates that improve cognitive deficits. PLoS One 2014, 9, e111898.
Guo, L., and Zhen, X. Sigma-2 receptor ligands: neurobiological effects. Curr Med Chem 2015, 22, 989-1003.
Alon, A., Schmidt, H. R., Wood, M. D., Sahn, J. J., Martin, S. F., and Kruse, A. C. Identification of the gene that codes for the sigma2 receptor. Proc Natl Acad Sci USA 2017, 114, 7160-7165.
Bartz, F., Kern, L., Erz, D., Zhu, M., Gilbert, D., Meinhof, T., Wirkner, U., Erfle, H., Muckenthaler, M., Pepperkok, R., and Runz, H. Identification of cholesterol-regulating genes by targeted RNAi screening. Cell Metab 2009, 10, 63-75.
Ebrahimi-Fakhari, D., Wahlster, L., Bartz, F., Werenbeck-Ueding, J., Praggastis, M., Zhang, J., Joggerst-Thomalla, B., Theiss, S., Grimm, D., Ory, D. S., and Runz, H. Reduction of TMEM97 increases NPC1 protein levels and restores cholesterol trafficking in Niemann-pick type C1 disease cells. Hum Mol Genet 2016, 25, 3588-3599.
Liu, C. C., Yu, C. F., Wang, S. C., Li, H. Y., Lin, C. M., Wang, H. H., Abate, C., and Chiang, C. S. Sigma-2 receptor/TMEM97 agonist PB221 as an alternative drug for brain tumor. BMC Cancer 2019, 19, 473.
Yang, K., Zeng, C., Wang, C., Sun, M., Yin, D., and Sun, T. Sigma-2 Receptor-A Potential Target for Cancer/Alzheimer's Disease Treatment via Its Regulation of Cholesterol Homeostasis. Molecules 2020, 25.
Sahn, J. J., Mejia, G. L., Ray, P. R., Martin, S. F., and Price, T. J. Sigma 2 Receptor/Tmem97 Agonists Produce Long Lasting Antineuropathic Pain Effects in Mice. ACS Chem Neurosci 2017, 8, 1801-1811.
Vazquez-Rosa, E., Watson, M. R., Sahn, J. J., Hodges, T. R., Schroeder, R. E., Cintron-Perez, C. J., Shin, M. K., Yin, T. C., Emery, J. L., Martin, S. F., Liebl, D. J., and Pieper, A. A. Neuroprotective Efficacy of a Sigma 2 Receptor/TMEM97 Modulator (DKR-1677) after Traumatic Brain Injury. ACS Chem Neurosci 2019, 10, 1595-1602.
Scott, L. L., Sahn, J. J., Ferragud, A., Yen, R. C., Satarasinghe, P. N., Wood, M. D., Hodges, T. R., Shi, T., Prakash, B. A., Friese, K. M., Shen, A., Sabino, V., Pierce, J. T., and Martin, S. F. Small molecule modulators of sigma2R/Tmem97 reduce alcohol withdrawal-induced behaviors. Neuropsychopharmacology 2018, 43, 1867-1875.
Quadir, S. G., Tanino, S. M., Rohl, C. D., Sahn, J. J., Yao, E. J., Cruz, L. D. R., Cottone, P., Martin, S. F., and Sabino, V. The Sigma-2 receptor/transmembrane protein 97 (sigma2R/TMEM97) modulator JVW-1034 reduces heavy alcohol drinking and associated pain states in male mice. Neuropharmacology 2021, 184, 108409.
Mondal, S., Hegarty, E., Sahn, J. J., Scott, L. L., Gokce, S. K., Martin, C., Ghorashian, N., Satarasinghe, P. N., Iyer, S., Sae-Lee, W., Hodges, T. R., Pierce, J. T., Martin, S. F., and Ben-Yakar, A. High-Content Microfluidic Screening Platform Used To Identify sigma2R/Tmem97 Binding Ligands that Reduce Age-Dependent Neurodegeneration in C. elegans SC_APP Model. ACS Chem Neurosci 2018, 9, 1014-1026.
Riad, A., Lengyel-Zhand, Z., Zeng, C., Weng, C. C., Lee, V. M., Trojanowski, J. Q., and Mach, R. H. The Sigma-2 Receptor/TMEM97, PGRMC1, and LDL Receptor Complex Are Responsible for the Cellular Uptake of Abeta42 and Its Protein Aggregates. Mol Neurobiol 2020, 57, 3803-3813.
Yi, B., Sahn, J. J., Ardestani, P. M., Evans, A. K., Scott, L. L., Chan, J. Z., Iyer, S., Crisp, A., Zuniga, G., Pierce, J. T., Martin, S. F., and Shamloo, M. Small molecule modulator of sigma 2 receptor is neuroprotective and reduces cognitive deficits and neuroinflammation in experimental models of Alzheimer's disease. J Neurochem 2017, 140, 561-575.
Izzo, N. J., Xu, J., Zeng, C., Kirk, M. J., Mozzoni, K., Silky, C., Rehak, C., Yurko, R., Look, G., Rishton, G., Safferstein, H., Cruchaga, C., Goate, A., Cahill, M. A., Arancio, O., Mach, R. H., Craven, R., Head, E., LeVine, H., 3rd, Spires-Jones, T. L., and Catalano, S. M. Alzheimer's therapeutics targeting amyloid beta 1-42 oligomers II: Sigma-2/PGRMC1 receptors mediate Abeta 42 oligomer binding and synaptotoxicity. PLoS One 2014, 9, e111899.
Grundman, M., Morgan, R., Lickliter, J. D., Schneider, L. S., DeKosky, S., Izzo, N. J., Guttendorf, R., Higgin, M., Pribyl, J., Mozzoni, K., Safferstein, H., and Catalano, S. M. A phase 1 clinical trial of the sigma-2 receptor complex allosteric antagonist CT1812, a novel therapeutic candidate for Alzheimer's disease. Alzheimers Dement (N Y) 2019, 5, 20-26.
Sahn, J. J., Hodges, T. R., Chan, J. Z., and Martin, S. F. Norbenzomorphan Framework as a Novel Scaffold for Generating Sigma 2 Receptor/PGRMC1 Subtype-Selective Ligands. ChemMedChem 2016, 11, 556-561.
Sahn, J. J., and Martin, S. F. Expedient synthesis of norbenzomorphan library via multicomponent assembly process coupled with ring-closing reactions. ACS Comb Sci 2012, 14, 496-502.
Sunderhaus, J. D., Dockendorff, C., and Martin, S. F. Synthesis of Diverse Heterocyclic Scaffolds via Tandem Additions to Imine Derivatives and Ring-Forming Reactions. Tetrahedron 2009, 65, 6454-6469.
Sahn, J. J., Hodges, T. R., Chan, J. Z., and Martin, S. F. Norbenzomorphan Scaffold: Chemical Tool for Modulating Sigma Receptor-Subtype Selectivity. ACS Med Chem Lett 2017, 8, 455-460.
Linkens, K., Schmidt, H. R., Sahn, J. J., Kruse, A. C., and Martin, S. F. Investigating isoindoline, tetrahydroisoquinoline, and tetrahydrobenzazepine scaffolds for their sigma receptor binding properties. Eur J Med Chem 2018, 151, 557-567.
Besnard, J., Ruda, G. F., Setola, V., Abecassis, K., Rodriguiz, R. M., Huang, X. P., Norval, S., Sassano, M. F., Shin, A. I., Webster, L. A., Simeons, F. R., Stojanovski, L., Prat, A., Seidah, N. G., Constam, D. B., Bickerton, G. R., Read, K. D., Wetsel, W. C., Gilbert, I. H., Roth, B. L., and Hopkins, A. L. Automated design of ligands to polypharmacological profiles. Nature 2012, 492, 215-220.
Arbez, N., Ratovitski, T., Roby, E., Chighladze, E., Stewart, J. C., Ren, M., Wang, X., Lavery, D. J., and Ross, C. A. Post-translational modifications clustering within proteolytic domains decrease mutant huntingtin toxicity. J Biol Chem 2017, 292, 19238-19249.
Hegde, R. N., Chiki, A., Petricca, L., Martufi, P., Arbez, N., Mouchiroud, L., Auwerx, J., Landles, C., Bates, G. P., Singh-Bains, M. K., Dragunow, M., Curtis, M. A., Faull, R. L., Ross, C. A., Caricasole, A., and Lashuel, H. A. TBK1 phosphorylates mutant Huntingtin and suppresses its aggregation and toxicity in Huntington's disease models. EMBO J 2020, 39, e104671.
Ogawa, S., Okuyama, S., Araki, H., and Otomo, S. Effect of NE-100, a novel sigma receptor ligand, on phencyclidine-induced cognitive dysfunction. Eur J Pharmacol 1994, 263, 9-15.
Narita, M., Yoshizawa, K., Aoki, K., Takagi, M., Miyatake, M., and Suzuki, T. A putative sigma1 receptor antagonist NE-100 attenuates the discriminative stimulus effects of ketamine in rats. Addict Biol 2001, 6, 373-376.
Tabrizi, S. J., Flower, M. D., Ross, C. A., and Wild, E. J. Huntington disease: new insights into molecular pathogenesis and therapeutic opportunities. Nat Rev Neurol 2020, 16, 529-546.
Ross, C. A. Huntington's disease: new paths to pathogenesis. Cell 2004, 118, 4-7.
Ross, C. A., and Tabrizi, S. J. Huntington's disease: from molecular pathogenesis to clinical treatment. Lancet Neurol 2011, 10, 83-98.
Saudou, F., and Humbert, S. The Biology of Huntingtin. Neuron 2016, 89, 910-926.
Barker, R. A., Fujimaki, M., Rogers, P., and Rubinsztein, D. C. Huntingtin-lowering strategies for Huntington's disease. Expert Opin Investig Drugs 2020, 29, 1125-1132.
Marxreiter, F., Stemick, J., and Kohl, Z. Huntingtin Lowering Strategies. Int J Mol Sci 2020, 21.

1. A compound of the formula: 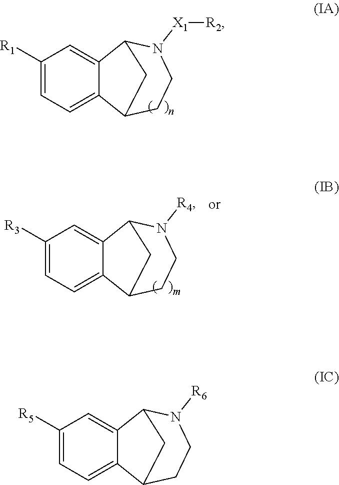
wherein:
m and n are each independently 1 or 2;
R₁ is —Z₁(R₁′)ₐ, wherein:
Z₁ is arenediyl_{(C≤18)}, substituted arenediyl_{(C≤18)}, heteroarenediyl_{(C≤18)}, substituted heteroarenediyl_{(C≤18)}, when a is 1; arenetriyl_{(C≤18)}, substituted arenetriyl_{(C≤18)}, heteroarenetriyl_{(C≤18)}, substituted heteroarenetriyl_{(C≤18)}, when a is 2; arenequadyl_{(C≤18)}, substituted arenequadyl_{(C≤18)}, heteroarenequadyl_{(C≤18)}, substituted heteroarenequadyl_{(C≤18)}, when a is 3; or arenepentayl_{(C≤18)}, substituted arenepentayl_{(C≤18)}, heteroarenepentayl_{(C≤18)}, substituted heteroarenepentayl_{(C≤18)}, when a is 4;
a is 1, 2, 3, or 4; and
R₁′ is hydrogen, —C(O)R′, —OR′, —S(O)_{y}R′, —N(R′)R″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
R′ and R″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof; and
y is 0, 1, or 2;
X₁ is —C(O)Y₁— or —S(O)ₓY₁—, wherein:
x is 0, 1, or 2; and
Y₁ is a covalent bond; —O—; —NRₐ—, wherein Rₐ is hydrogen, alkyl_{(C≤6)}, or substituted alkyl_{(C≤6)}; alkanediyl_{(C≤8)}; or substituted alkanediyl_{(C≤8)}; and
R₂ is —Y₂N(R_{b})R_{c}, wherein:
Y₂ is alkanediyl_{(C≤8)}, cycloalkanediyl_{(C≤8)}, alkenediyl_{(C≤8)}, alkynediyl_{(C≤8)}, arenediyl_{(C≤8)}, heterocycloalkanediyl_{(C≤8)}, heteroarenediyl_{(C≤8)}, or a substituted version thereof;
R_{b} and R_{c} are each independently hydrogen, alkyl_{(C≤12)}, cycloalkyl_{(C≤12)}, aryl_{(C≤12)}, heteroaryl_{(C≤12)}, heterocycloalkyl_{(C≤12)}, acyl_{(C≤12)}, or a substituted version thereof; or
R₂ is —Y₃R_{d}, wherein:
Y₃ is heterocycloalkanediyl_{(C≤8)}, heteroarenediyl_{(C≤8)}, or a substituted version thereof; and
R_{d} is hydrogen, alkyl_{(C≤12)}, alkenyl_{(C≤12)}, alkynyl_{(C≤12)}, aryl_{(C≤12)}, heteroaryl_{(C≤12)}, heterocycloalkyl_{(C≤12)}, or a substituted version thereof;
R₃ is —Y₄(Rₑ)_{b}, wherein:
Y₄ is arenediyl_{(C≤18)}, substituted arenediyl_{(C≤18)}, heteroarenediyl_{(C≤18)}, substituted heteroarenediyl_{(C≤18)}, wherein b is 1; arenetriyl_{(C≤18)}, substituted arenetriyl_{(C≤18)}, heteroarenetriyl_{(C≤18)}, substituted heteroarenetriyl_{(C≤18)}, wherein b is 2; arenequadyl_{(C≤18)}, substituted arenequadyl_{(C≤18)}, heteroarenequadyl_{(C≤18)}, substituted heteroarenequadyl_{(C≤18)}, wherein b is 3; or arenepentayl_{(C≤18)}, substituted arenepentayl_{(C≤18)}, heteroarenepentayl_{(C≤18)}, substituted heteroarenepentayl_{(C≤18)}, wherein b is 4;
b is 1, 2, 3, or 4; and
Rₑ is hydrogen, —C(O)Rₑ′, —ORₑ′, —S(O)_{y}Rₑ′, —N(Rₑ′)Rₑ″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
y is 0, 1, or 2; and
Rₑ′ and Rₑ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
R₄ is —Y₅N(R_{f})R_{g}, wherein:
Y₅ is cycloalkanediyl_{(C≤12)}, heterocycloalkyl_{(C≤12)}, or a substituted version of either group;
R_{f} and R_{g} are each independently hydrogen, alkyl_{(C≤12)}, cycloalkyl_{(C≤12)}, aryl_{(C≤12)}, heteroaryl_{(C≤12)}, heterocycloalkyl_{(C≤12)}, acyl_{(C≤12)}, or a substituted version thereof; or
R₄ is —Y₆R₇, wherein:
Y₆ is alkanediyl_{(C≤12)} or a substituted version thereof; and
R₇ is —C(O)Rₕ′, —S(O)_{z}Rₕ′, —N(Rₕ′)Rₕ″, heterocycloalkyl_{(C≤12)}, or substituted heterocycloalkyl_{(C≤12)}; wherein:
z is 0, 1, or 2; and
Rₕ′ and Rₕ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
R₅ is —Y₇(Rᵢ)_{c}, wherein:
Y₇ is arenediyl_{(C≤18)}, substituted arenediyl_{(C≤18)}, heteroarenediyl_{(C≤18)}, substituted heteroarenediyl_{(C≤18)}, wherein c is 1, arenetriyl_{(C≤18)}, substituted arenetriyl_{(C≤18)}, heteroarenetriyl_{(C≤18)}, substituted heteroarenetriyl_{(C≤18)}, wherein c is 2; arenequadyl_{(C≤18)}, substituted arenequadyl_{(C≤18)}, heteroarenequadyl_{(C≤18)}, substituted heteroarenequadyl_{(C≤18)}, wherein c is 3; or arenepentayl_{(C≤18)}, substituted arenepentayl_{(C≤18)}, heteroarenepentayl_{(C≤18)}, substituted heteroarenepentayl_{(C≤18)}, wherein c is 4;
c is 1, 2, 3, or 4; and
Rᵢ is hydrogen, —C(O)Rᵢ′, —ORᵢ′, —S(O)ᵥRᵢ′, —N(Rᵢ′)Rᵢ″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
v is 0, 1, or 2; and
Rᵢ′ and Rᵢ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
R₆ is —Y₇R₈, wherein:
Y₇ is alkanediyl_{(C≤12)} or a substituted version thereof; and
R₈ is —ORⱼ′, wherein:
Rⱼ′ is hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
or a pharmaceutically acceptable salt thereof.

2. The compound of claim 1, wherein the compound is further defined as: 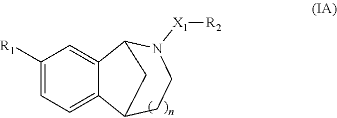
wherein:
n is 1 or 2;
R₁ is —Z₁(R₁′)ₐ, wherein:
Z₁ is arenediyl_{(C≤18)}, substituted arenediyl_{(C≤18)}, heteroarenediyl_{(C≤18)}, substituted heteroarenediyl_{(C≤18)}, when a is 1; arenetriyl_{(C≤18)}, substituted arenetriyl_{(C≤18)}, heteroarenetriyl_{(C≤18)}, substituted heteroarenetriyl_{(C≤18)}, when a is 2; arenequadyl_{(C≤18)}, substituted arenequadyl_{(C≤18)}, heteroarenequadyl_{(C≤18)}, substituted heteroarenequadyl_{(C≤18)}, when a is 3; or arenepentayl_{(C≤18)}, substituted arenepentayl_{(C≤18)}, heteroarenepentayl_{(C≤18)}, substituted heteroarenepentayl_{(C≤18)}, when a is 4;
a is 1, 2, 3, or 4; and
R₁′ is hydrogen, —C(O)R′, —OR′, —S(O)_{y}R′, —N(R′)R″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
R′ and R″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof; and
y is 0, 1, or 2;
X₁ is —C(O)Y₁— or —S(O)ₓY₁—, wherein:
x is 0, 1, or 2; and
Y₁ is a covalent bond, —O—; —NRₐ—, wherein Rₐ is hydrogen, alkyl_{(C≤6)}, or substituted alkyl_{(C≤6)}; alkanediyl_{(C≤8)}; or substituted alkanediyl_{(C≤8)}; and
R₂ is —Y₂N(R_{b})R_{c}, wherein:
Y₂ is alkanediyl_{(C≤8)}, cycloalkanediyl_{(C≤8)}, alkenediyl_{(C≤8)}, alkynediyl_{(C≤8)}, arenediyl_{(C≤8)}, heterocycloalkanediyl_{(C≤8)}, heteroarenediyl_{(C≤8)}, or a substituted version thereof;
R_{b} and R_{c} are each independently hydrogen, alkyl_{(C≤12)}, cycloalkyl_{(C≤12)}, aryl_{(C≤12)}, heteroaryl_{(C≤12)}, heterocycloalkyl_{(C≤12)}, acyl_{(C≤12)}, or a substituted version thereof; or
R₂ is —Y₃R_{d}, wherein:
Y₃ is heterocycloalkanediyl_{(C≤8)}, heteroarenediyl_{(C≤8)}, or a substituted version thereof; and
R_{d} is hydrogen, alkyl_{(C≤12)}, alkenyl_{(C≤12)}, alkynyl_{(C≤12)}, aryl_{(C≤12)}, heteroaryl_{(C≤12)}, heterocycloalkyl_{(C≤12)}, or a substituted version thereof;
or a pharmaceutically acceptable salt thereof.

3. The compound of claim 1, wherein the compound is further defined as: 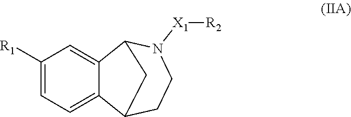
wherein:
R₁ is —Z₁(R₁′)ₐ, wherein:
Z₁ is arenediyl_{(C≤18)}, substituted arenediyl_{(C≤18)}, heteroarenediyl_{(C≤18)}, substituted heteroarenediyl_{(C≤18)}, when a is 1; arenetriyl_{(C≤18)}, substituted arenetriyl_{(C≤18)}, heteroarenetriyl_{(C≤18)}, substituted heteroarenetriyl_{(C≤18)}, when a is 2; arenequadyl_{(C≤18)}, substituted arenequadyl_{(C≤18)}, heteroarenequadyl_{(C≤18)}, substituted heteroarenequadyl_{(C≤18)}, when a is 3; or arenepentayl_{(C≤18)}, substituted arenepentayl_{(C≤18)}, heteroarenepentayl_{(C≤18)}, substituted heteroarenepentayl_{(C≤18)}, when a is 4;
a is 1, 2, 3, or 4; and
R₁′ is hydrogen, —C(O)R′, —OR′, —S(O)_{y}R′, —N(R′)R″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
R′ and R″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino (C=8), or a substituted version thereof; and
y is 0, 1, or 2;
X₁ is —C(O)Y₁— or —S(O)ₓY₁—, wherein:
x is 0, 1, or 2; and
Y₁ is a covalent bond, —O—; —NRₐ—, wherein Rₐ is hydrogen, alkyl_{(C≤6)}, or substituted alkyl_{(C≤6)}; alkanediyl_{(C≤8)}; or substituted alkanediyl_{(C≤8)}; and
R₂ is —Y₂N(R_{b})R_{c}, wherein:
Y₂ is alkanediyl_{(C≤8)}, cycloalkanediyl_{(C≤8)}, alkenediyl_{(C≤8)}, alkynediyl_{(C≤8)}, arenediyl_{(C≤8)}, heterocycloalkanediyl_{(C≤8)}, heteroarenediyl_{(C≤8)}, or a substituted version thereof;
R_{b} and R_{c} are each independently hydrogen, alkyl_{(C≤12)}, cycloalkyl_{(C≤12)}, aryl_{(C≤12)}, heteroaryl_{(C≤12)}, heterocycloalkyl_{(C≤12)}, acyl_{(C≤12)}, or a substituted version thereof; or
R₂ is —Y₃R_{d}, wherein:
Y₃ is heterocycloalkanediyl_{(C≤8)}, heteroarenediyl_{(C≤8)}, or a substituted version thereof; and
R_{d} is hydrogen, alkyl_{(C≤12)}, alkenyl_{(C≤12)}, alkynyl_{(C≤12)}, aryl_{(C≤12)}, heteroaryl_{(C≤12)}, heterocycloalkyl_{(C≤12)}, or a substituted version thereof;
or a pharmaceutically acceptable salt thereof.

4. The compound of claim 1, wherein the compound is further defined as: 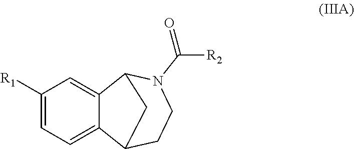
wherein:
R₁ is —Z₁(R₁′)ₐ, wherein:
Z₁ is arenediyl_{(C≤18)}, substituted arenediyl_{(C≤18)}, heteroarenediyl_{(C≤18)}, substituted heteroarenediyl_{(C≤18)}, when a is 1; arenetriyl_{(C≤18)}, substituted arenetriyl_{(C≤18)}, heteroarenetriyl_{(C≤18)}, substituted heteroarenetriyl_{(C≤18)}, when a is 2; arenequadyl_{(C≤18)}, substituted arenequadyl_{(C≤18)}, heteroarenequadyl_{(C≤18)}, substituted heteroarenequadyl_{(C≤18)}, when a is 3; or arenepentayl_{(C≤18)}, substituted arenepentayl_{(C≤18)}, heteroarenepentayl_{(C≤18)}, substituted heteroarenepentayl_{(C≤18)}, when a is 4;
a is 1, 2, 3, or 4; and
R₁′ is hydrogen, —C(O)R′, —OR′, —S(O)_{y}R′, —N(R′)R″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
R′ and R″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof; and
y is 0, 1, or 2;
R₂ is —Y₂N(R_{b})R_{c}, wherein:
Y₂ is alkanediyl_{(C≤8)}, cycloalkanediyl_{(C≤8)}, alkenediyl_{(C≤8)}, alkynediyl_{(C≤8)}, arenediyl_{(C≤8)}, heterocycloalkanediyl_{(C≤8)}, heteroarenediyl_{(C≤8)}, or a substituted version thereof;
R_{b} and R_{c} are each independently hydrogen, alkyl_{(C≤12)}, cycloalkyl_{(C≤12)}, aryl_{(C≤12)}, heteroaryl_{(C≤12)}, heterocycloalkyl_{(C≤12)}, acyl_{(C≤12)}, or a substituted version thereof; or
R₂ is —Y₃R_{d}, wherein:
Y₃ is heterocycloalkanediyl_{(C≤8)}, heteroarenediyl_{(C≤8)}, or a substituted version thereof; and
R_{d} is hydrogen, alkyl_{(C≤12)}, alkenyl_{(C≤12)}, alkynyl_{(C≤12)}, aryl_{(C≤12)}, heteroaryl_{(C≤12)}, heterocycloalkyl_{(C≤12)}, or a substituted version thereof;
or a pharmaceutically acceptable salt thereof.

5. The compound of claim 1, wherein the compound is further defined as: 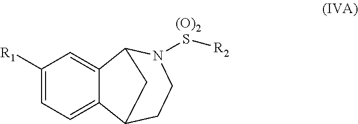
wherein:
R₁ is —Z₁(R₁′)ₐ, wherein:
Z₁ is arenediyl_{(C≤18)}, substituted arenediyl_{(C≤18)}, heteroarenediyl_{(C≤18)}, substituted heteroarenediyl_{(C≤18)}, when a is 1; arenetriyl_{(C≤18)}, substituted arenetriyl_{(C≤18)}, heteroarenetriyl_{(C≤18)}, substituted heteroarenetriyl_{(C≤18)}, when a is 2; arenequadyl_{(C≤18)}, substituted arenequadyl_{(C≤18)}, heteroarenequadyl_{(C≤18)}, substituted heteroarenequadyl_{(C≤18)}, when a is 3; or arenepentayl_{(C≤18)}, substituted arenepentayl_{(C≤18)}, heteroarenepentayl_{(C≤18)}, substituted heteroarenepentayl_{(C≤18)}, when a is 4;
a is 1, 2, 3, or 4; and
R₁′ is hydrogen, —C(O)R′, —OR′, —S(O)_{y}R′, —N(R′)R″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
R′ and R″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof; and
y is 0, 1, or 2;
R₂ is —Y₂N(R_{b})R_{c}, wherein:
Y₂ is alkanediyl_{(C≤8)}, cycloalkanediyl_{(C≤8)}, alkenediyl_{(C≤8)}, alkynediyl_{(C≤8)}, arenediyl_{(C≤8)}, heterocycloalkanediyl_{(C≤8)}, heteroarenediyl_{(C≤8)}, or a substituted version thereof;
R_{b} and R_{c} are each independently hydrogen, alkyl_{(C≤12)}, cycloalkyl_{(C≤12)}, aryl_{(C≤12)}, heteroaryl_{(C≤12)}, heterocycloalkyl_{(C≤12)}, acyl_{(C≤12)}, or a substituted version thereof; or
R₂ is —Y₃R_{d}, wherein:
Y₃ is heterocycloalkanediyl_{(C≤8)}, heteroarenediyl_{(C≤8)}, or a substituted version thereof; and
R_{d} is hydrogen, alkyl_{(C≤12)}, alkenyl_{(C≤12)}, alkynyl_{(C≤12)}, aryl_{(C≤12)}, heteroaryl_{(C≤12)}, heterocycloalkyl_{(C≤12)}, or a substituted version thereof;
or a pharmaceutically acceptable salt thereof.

6. The compound of claim 1 further defined as: 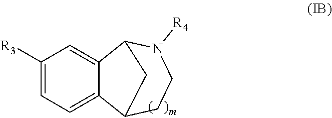
wherein:
m is 1 or 2;
R₃ is —Y₄(Rₑ)_{b}, wherein:
Y₄ is arenediyl_{(C≤18)}, substituted arenediyl_{(C≤18)}, heteroarenediyl_{(C≤18)}, substituted heteroarenediyl_{(C≤18)}, wherein b is 1; arenetriyl_{(C≤18)}, substituted arenetriyl_{(C≤18)}, heteroarenetriyl_{(C≤18)}, substituted heteroarenetriyl_{(C≤18)}, wherein b is 2; arenequadyl_{(C≤18)}, substituted arenequadyl_{(C≤18)}, heteroarenequadyl_{(C≤18)}, substituted heteroarenequadyl_{(C≤18)}, wherein b is 3; or arenepentayl_{(C≤18)}, substituted arenepentayl_{(C≤18)}, heteroarenepentayl_{(C≤18)}, substituted heteroarenepentayl_{(C≤18)}, wherein b is 4;
b is 1, 2, 3, or 4; and
Rₑ is hydrogen, —C(O)Rₑ′, —ORₑ′, —S(O)_{y}Rₑ′, —N(Rₑ′)Rₑ″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
y is 0, 1, or 2; and
Rₑ′ and Rₑ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
R₄ is —Y₅N(R_{f})R_{g}, wherein:
Y₅ is cycloalkanediyl_{(C≤12)}, heterocycloalkyl_{(C≤12)}, or a substituted version of either group;
R_{f} and R_{g} are each independently hydrogen, alkyl_{(C≤12)}, cycloalkyl_{(C≤12)}, aryl_{(C≤12)}, heteroaryl_{(C≤12)}, heterocycloalkyl_{(C≤12)}, acyl_{(C≤12)}, or a substituted version thereof; or
R₄ is —Y₆R₇, wherein:
Y₆ is alkanediyl_{(C≤12)} or a substituted version thereof; and
R₇ is —C(O)Rₕ′, —S(O)_{z}Rₕ′, —N(Rₕ′)Rₕ″, heterocycloalkyl_{(C≤12)}, or substituted heterocycloalkyl_{(C≤12)}; wherein:
z is 0, 1, or 2; and
Rₕ′ and Rₕ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
or a pharmaceutically acceptable salt thereof.

7. The compound of claim 1 further defined as: 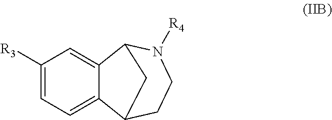
wherein:
R₃ is —Y₄(Rₑ)_{b}, wherein:
Y₄ is arenediyl_{(C≤18)}, substituted arenediyl_{(C≤18)}, heteroarenediyl_{(C≤18)}, substituted heteroarenediyl_{(C≤18)}, wherein b is 1; arenetriyl_{(C≤18)}, substituted arenetriyl_{(C≤18)}, heteroarenetriyl_{(C≤18)}, substituted heteroarenetriyl_{(C≤18)}, wherein b is 2; arenequadyl_{(C≤18)}, substituted arenequadyl_{(C≤18)}, heteroarenequadyl_{(C≤18)}, substituted heteroarenequadyl_{(C≤18)}, wherein b is 3; or arenepentayl_{(C≤18)}, substituted arenepentayl_{(C≤18)}, heteroarenepentayl_{(C≤18)}, substituted heteroarenepentayl_{(C≤18)}, wherein b is 4;
b is 1, 2, 3, or 4; and
Rₑ is hydrogen, —C(O)Rₑ′, —ORₑ′, —S(O)_{y}Rₑ′, —N(Rₑ′)Rₑ″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
y is 0, 1, or 2; and
Rₑ′ and Rₑ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
R₄ is —Y₆R₇, wherein:
Y₆ is alkanediyl_{(C≤12)} or a substituted version thereof; and
R₇ is —C(O)Rₕ′, —S(O)_{z}Rₕ′, —N(Rₕ′)Rₕ″, heterocycloalkyl_{(C≤12)}, or substituted heterocycloalkyl_{(C≤12)}; wherein:
z is 0, 1, or 2; and
Rₕ′ and Rₕ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
or a pharmaceutically acceptable salt thereof.

8. The compound of claim 1 further defined as: 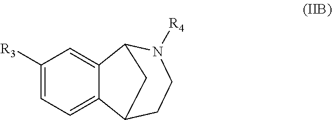
wherein:
R₃ is —Y₄(Rₑ)_{b}, wherein:
Y₄ is arenediyl_{(C≤18)}, substituted arenediyl_{(C≤18)}, heteroarenediyl_{(C≤18)}, substituted heteroarenediyl_{(C≤18)}, wherein b is 1; arenetriyl_{(C≤18)}, substituted arenetriyl_{(C≤18)}, heteroarenetriyl_{(C≤18)}, substituted heteroarenetriyl_{(C≤18)}, wherein b is 2; arenequadyl_{(C≤18)}, substituted arenequadyl_{(C≤18)}, heteroarenequadyl_{(C≤18)}, substituted heteroarenequadyl_{(C≤18)}, wherein b is 3; or arenepentayl_{(C≤18)}, substituted arenepentayl_{(C≤18)}, heteroarenepentayl_{(C≤18)}, substituted heteroarenepentayl_{(C≤18)}, wherein b is 4;
b is 1, 2, 3, or 4; and
Rₑ is hydrogen, —C(O)Rₑ′, —ORₑ′, —S(O)_{y}Rₑ′, —N(Rₑ′)Rₑ″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
y is 0, 1, or 2; and
Rₑ′ and Rₑ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
R₄ is —Y₅N(R_{f})R_{g}, wherein:
Y₅ is cycloalkanediyl_{(C≤12)}, heterocycloalkyl_{(C≤12)}, or a substituted version of either group;
R_{f} and R_{g} are each independently hydrogen, alkyl_{(C≤12)}, cycloalkyl_{(C≤12)}, aryl_{(C≤12)}, heteroaryl_{(C≤12)}, heterocycloalkyl_{(C≤12)}, acyl_{(C≤12)}, or a substituted version thereof; or
or a pharmaceutically acceptable salt thereof.

9. The compound of claim 1 further defined as: 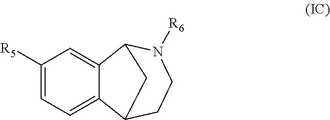
wherein:
R₅ is —Y₇(Rᵢ)_{c}, wherein:
Y₇ is arenediyl_{(C≤18)}, substituted arenediyl_{(C≤18)}, heteroarenediyl_{(C≤18)}, substituted heteroarenediyl_{(C≤18)}, wherein c is 1, arenetriyl_{(C≤18)}, substituted arenetriyl_{(C≤18)}, heteroarenetriyl_{(C≤18)}, substituted heteroarenetriyl_{(C≤18)}, wherein c is 2; arenequadyl_{(C≤18)}, substituted arenequadyl_{(C≤18)}, heteroarenequadyl_{(C≤18)}, substituted heteroarenequadyl_{(C≤18)}, wherein c is 3; or arenepentayl_{(C≤18)}, substituted arenepentayl_{(C≤18)}, heteroarenepentayl_{(C≤18)}, substituted heteroarenepentayl_{(C≤18)}, wherein c is 4;
c is 1, 2, 3, or 4; and
Rᵢ is hydrogen, —C(O)Rᵢ′, —ORᵢ′, —S(O)ᵥRᵢ′, —N(Rᵢ′)Rᵢ″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein:
v is 0, 1, or 2; and
Rᵢ′ and Rᵢ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
R₆ is alkyl_{(C≤12)}, substituted alkyl_{(C≤12)}, cycloalkyl_{(C≤12)}, substituted cycloalkyl_{(C≤12)}, —Y₇R₈, wherein:
Y₇ is alkanediyl_{(C≤12)} or a substituted version thereof; and
R₈ is —ORⱼ′, wherein:
Rⱼ′ is hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof;
or a pharmaceutically acceptable salt thereof.

10.-16. (canceled)

17. The compound of claim 1, wherein R₁ is —Z₁(R₁′)ₐ, wherein: Z₁ is arenediyl_{(C≤18)}, substituted arenediyl_{(C≤18)}, heteroarenediyl_{(C≤18)}, substituted heteroarenediyl_{(C≤18)}, when a is 1; arenetriyl_{(C≤18)}, substituted arenetriyl_{(C≤18)}, heteroarenetriyl_{(C≤18)}, substituted heteroarenetriyl_{(C≤18)}, when a is 2; or arenequadyl_{(C≤18)}, substituted arenequadyl_{(C≤18)}, heteroarenequadyl_{(C≤18)}, substituted heteroarenequadyl_{(C≤18)}, when a is 3; a is 1, 2, or 3; and Rᵢ′ is hydrogen, —C(O)R′, —OR′, —S(O)_{y}R′, —N(R′)R″, cyano, halo, alkyl_{(C≤8)}, substituted alkyl_{(C≤8)}, cycloalkyl_{(C≤8)}, substituted heterocycloalkyl_{(C≤8)}, or substituted heterocycloalkyl_{(C≤8)}; wherein: R′ and R″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof; and y is 0, 1, or 2.

18.-48. (canceled)

49. The compound of claim 1, wherein R₂ is —Y₂N(R_{b})R_{c}.

50.-60. (canceled)

61. The compound of claim 1, wherein R₂ is —Y₃R_{d}.

62.-70. (canceled)

71. The compound of claim 1, wherein R₃ is —Y₄(Rₑ)_{b}, wherein: Y₄ is arenediyl_{(C≤12)}, substituted arenediyl_{(C≤12)}, arenetriyl_{(C≤18)}, substituted arenetriyl_{(C≤18)}, arenequadyl_{(C≤18)}, or substituted arenequadyl_{(C≤18)}.

72.-101. (canceled)

102. The compound of claim 1, wherein R₄ is —Y₅N(R_{f})R_{g}, wherein: Y₅ is cycloalkanediyl_{(C≤12)} or substituted cycloalkanediyl_{(C≤12)}.

103.-112. (canceled)

113. The compound of claim 1, wherein R₄ is —Y₆R₇, wherein: Y₆ is alkanediyl_{(C≤12)} or a substituted version thereof; and R₇ is —N(Rₕ′)Rₕ″, heterocycloalkyl_{(C≤12)}, or substituted heterocycloalkyl_{(C≤12)}; wherein: z is 0, 1, or 2; and Rₕ′ and Rₕ″ are each independently hydrogen, alkyl_{(C≤8)}, or a substituted version thereof.

114. The compound of claim 1, wherein R₅ is —Y₇(Rᵢ)_{c}, wherein: Y₇ is arenediyl_{(C≤18)}, substituted arenediyl_{(C≤18)}, heteroarenediyl_{(C≤18)}, substituted heteroarenediyl_{(C≤18)}, wherein c is 1; arenetriyl_{(C≤18)}, substituted arenetriyl_{(C≤18)}, heteroarenetriyl_{(C≤18)}, substituted heteroarenetriyl_{(C≤18)}, wherein c is 2; arenequadyl_{(C≤18)}, substituted arenequadyl_{(C≤18)}, heteroarenequadyl_{(C≤18)}, substituted heteroarenequadyl_{(C≤18)}, wherein c is 3; c is 1, 2, or 3; and Rᵢ is hydrogen, —C(O)Rᵢ′, —ORᵢ′, —S(O)ᵥRᵢ′, —N(Rᵢ′)Rᵢ″, cyano, halo, alkyl_{(C≤8)}, substituted alkyl_{(C≤8)}, cycloalkyl_{(C≤8)}, substituted cycloalkyl_{(C≤8)}, heterocycloalkyl_{(C≤8)}, or substituted heterocycloalkyl_{(C≤8)}; wherein: v is 0, 1, or 2; and Rᵢ′ and Rᵢ″ are each independently hydrogen, alkyl_{(C≤8)}, alkenyl_{(C≤8)}, alkynyl_{(C≤8)}, aryl_{(C≤8)}, aralkyl_{(C≤8)}, heteroaryl_{(C≤8)}, heteroaralkyl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, or a substituted version thereof.

115.-144. (canceled)

145. The compound of claim 1, wherein R₆ is —Y₇R₈, wherein: Y₇ is alkanediyl_{(C≤12)} or a substituted version thereof; and R₈ is —ORⱼ′, wherein: Rⱼ′ is hydrogen, alkyl_{(C≤8)}, or a substituted version thereof.

146.-148. (canceled)

149. The compound of claim 1, wherein the compound is further defined as: 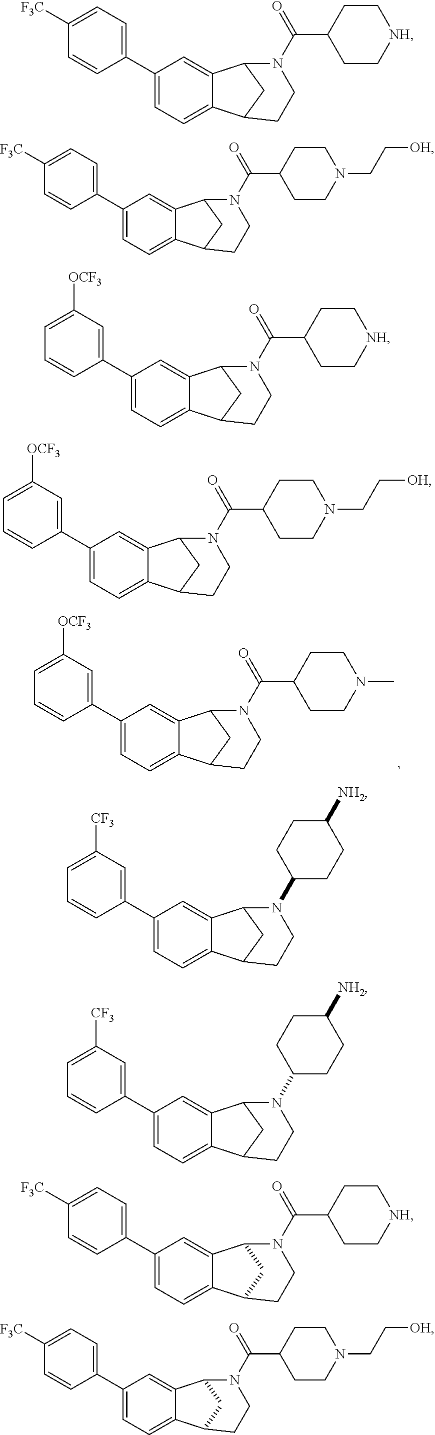 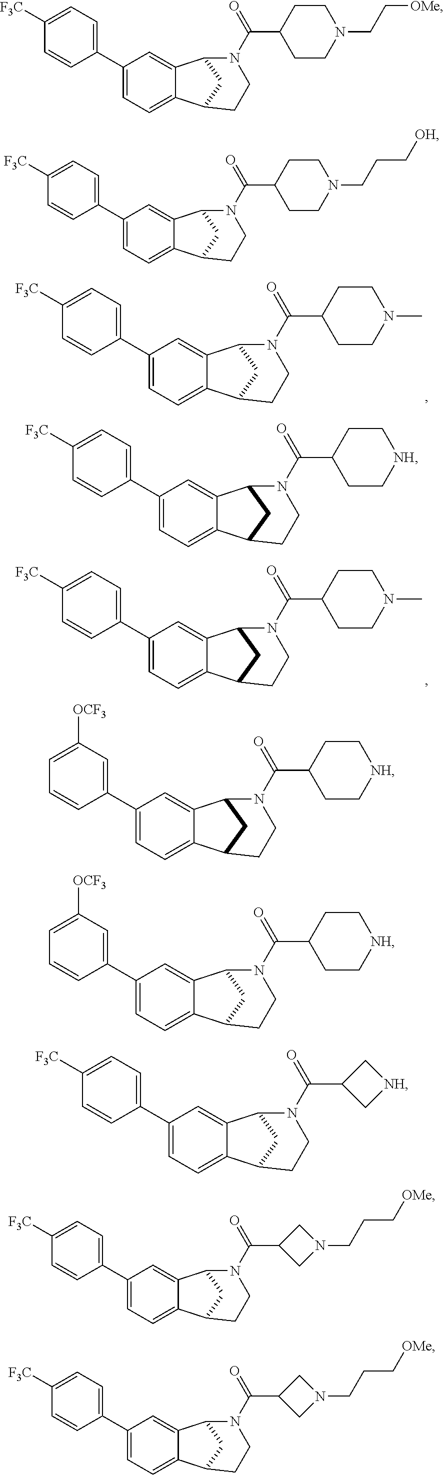 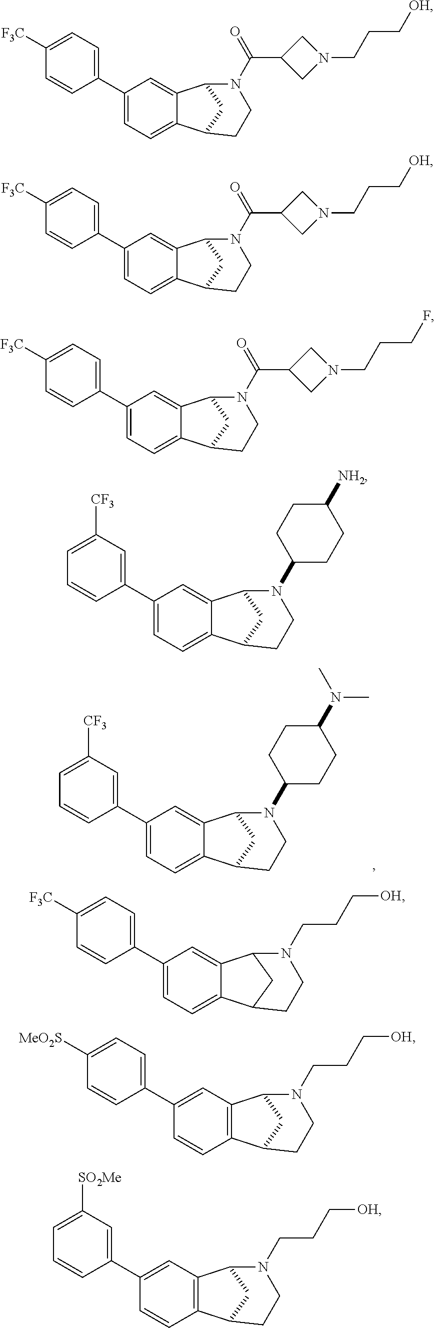 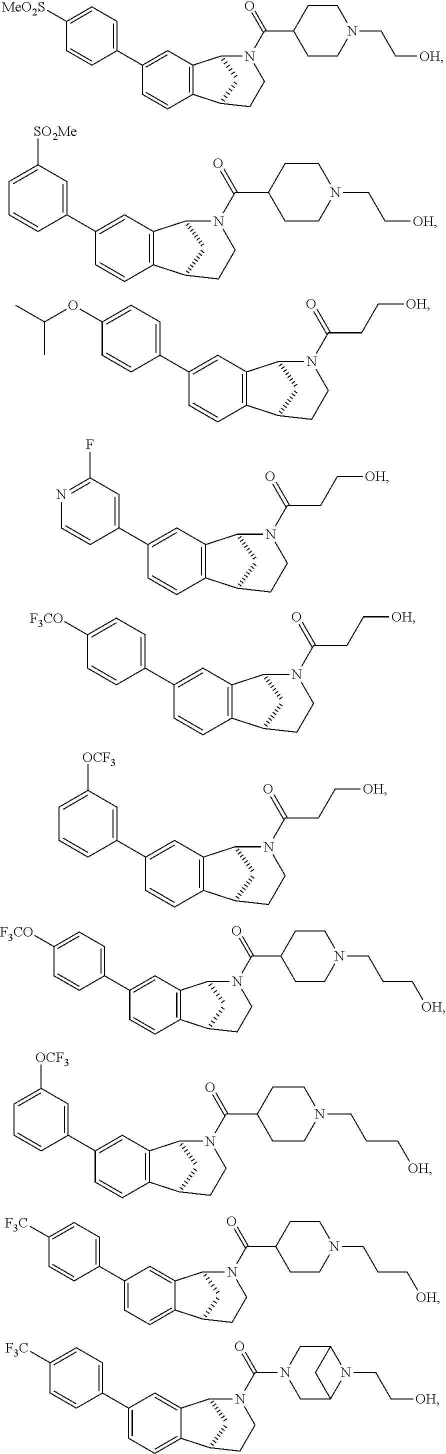 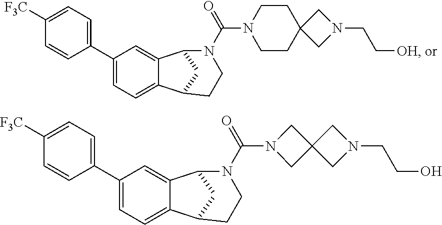
or a pharmaceutically acceptable salt thereof.

150. A pharmaceutical composition comprising:
(A) a compound of claim 1; and
(B) an excipient.

151.-154. (canceled)

155. A method of treating a disease or disorder in a patient comprising administering to the patient in need thereof a therapeutically effective amount of a compound of claim 1.

156.-165. (canceled)


SEQUENCE LISTING (2 sequences)

SEQ ID NO: 1 - DNA, 528 bp, Homo sapiens
  source 1..528: mol_type = other DNA
atgggggctccggcaaccaggcgctgcgtggagtggctgctgggcctctacttcctcagccacatccccatcaccctgttcatggacctgcaggcggtgctgccgcgcgagctctacccagtcgagtttagaaacctgctgaagtggtatgctaaggagttcaaagacccactgctacaggagcccccagcctggtttaagtcctttctgttttgcgagcttgtgtttcagctgcctttctttcccattgcaacgtatgccttcctcaaaggaagctgcaagtggattcgaactcctgcaatcatctactctgttcacaccatgacaaccttaattccgatactctccacatttctgtttgaggatttctccaaagccagtggtttcaagggacaaagacctgagactttgcatgaacggttaacccttgtgtctgtctatgccccctacttactcatcccattcatacttttaattttcatgttgcggagcccctactacaagtatgaagagaaaagaaaaaaaaaa

SEQ ID NO: 2 - protein, 176 aa, Homo sapiens
MGAPATRRCVEWLLGLYFLSHIPITLFMDLQAVLPRELYPVEFRNLLKWYAKEFKDPLLQEPPAWFKSFLFCELVFQLPFFPIATYAFLKGSCKWIRTPAIIYSVHTMTTLIPILSTFLFEDFSKASGFKGQRPETLHERLTLVSVYAPYLLIPFILLIFMLRSPYYKYEEKRKKK